SEQUENCE LISTING (3 sequences)

SEQ ID NO: 1 - protein, 14 aa, Homo sapiens
  DISULFID (3)..(14): Disulfide link between cysteines
AGCKNFFWKTFTSC

SEQ ID NO: 2 - protein, 28 aa, Homo sapiens
  DISULFID (17)..(28): Disulfide link between cysteines
SANSNPAMAPRERKAGCKNFFWKTFTSC

SEQ ID NO: 3 - protein, 8 aa, Artificial
  Synthetic
  DISULFID (2)..(7): Disulfide link between cysteines
ACYWKVCT

(19) United States
(12) Patent Application Publication
(10) Pub. No.: US 2024/0131116 A1
(43) Pub. Date: Apr. 25, 2024

(54) METHODS AND THREAPEUTIC COMBINATIONS FOR TREATING IDIOPATHIC INTRACRANIAL HYPERTENSION AND CLUSTER HEADACHES

(71) Applicant: NBO PHARMA LLC, Bedford, NY (US)

(72) Inventors: Fredric D. PRICE, Bedford, NY (US); Barry R. FRANKEL, New York, NY (US); Dorian BEVEC, Bad Nauheim (DE); Warren WASIEWSKI, Lancaster, PA (US)

(73) Assignee: NBO PHARMA LLC, Bedford, NY (US)

(21) Appl. No.: 18/276,724

(22) PCT Filed: Feb. 23, 2022
(86) PCT No.: PCT/US2022/017551

§ 371 Date: Aug. 10, 2023

Related U.S. Application Data
(60) Provisional application No. 63/153,122, filed on Feb. 24, 2021.

(51) Int. Cl. A61K 38/31; A61K 9/00; A61K 31/18; A61K 31/423; A61K 31/433; A61K 31/54; A61K 31/7048; A61P 25/00

(52) U.S. Cl. CPC A61K 38/31; A61K 9/0043; A61K 31/18; A61K 31/423; A61K 31/433; A61K 31/54; A61K 31/7048; A61P 25/00

(57) ABSTRACT

Methods and therapeutic combinations are provided for treating idiopathic intracranial hypertension (IIH) and cluster headache, comprising administering a somatostatin mimetic such as octreotide formulated for direct nose-to-brain administration, and a carbonic anhydrase inhibitor such as topiramate or acetazolamide, at a dose level and/or dosing regimen lower than effective when administered alone.

### RELATED APPLICATION

This application claims the benefit of and priority to U.S. Provisional Application No. 63/153,122, filed Feb. 24, 2021, the entire contents of which are hereby incorporated by reference.

### BACKGROUND

Headache caused by idiopathic intracranial hypertension (IIH), cluster headaches and other related conditions are often debilitating and for which effective and easily administrable treatments are lacking. Moreover, such conditions can lead to permanent deficits; for example, the increased intracranial pressure (ICP) in IIH can damage the optic nerve resulting in permanent vision loss. At minimum, these conditions significantly impact quality of life.

Systemic medications, life-style changes, and even surgical procedures are used to treat such conditions. Effective treatments that are non-invasive, easily compliant, safe and facile to administer are needed.

Octreotide is a somatostatin mimetic (also referred to as a somatostatin receptor agonist) typically administered parenterally (subcutaneously or intramuscularly) for treatment of acromegaly; an oral form has recently been approved for treatment of acromegaly (MYCAPSSA), and intranasal forms have also been tested (e.g., Weeke et al., J Clin Endocrin Metabol 1992; 75(1):163-169; Harris et al., Metabolism 1992 September; 41(9), Suppl. 2:72-75; Kissel et al., Pharmaceutical Research 1992; 9(1):52-57; Kaal et al., Eur J Endocrinol 2000; 143:353-361; Kokare et al., Pharm Anal Acta 2018, 9:43; Invetti et al., Endocrinol. Invest. 1996; 19:548-555; and Oechslein et al., Int J Pharmaceutics 1996; 139:25-32).

Octreotide administered subcutaneously has been evaluated in treating IIH (e.g., Anteraki et al., Lancet 1993 Nov. 6; 342(8880):1170; Matharu et al., Ann Neurol 2004; 56:488-494; Panagopoulos et al., Neurol Neurophysiol Neurosci 2007; 10: 1; Deftereos et al., Cephalalgia 2011; 31(16) 1679-1680; House et al., Clin Neural Neurosurg. 2016 November; 150:181-184; Manti et al., The Child 2013; 1(1):11-12). However, repeated injections are undesirable. While oral dosing eliminates the pain and discomfort of injections, its timing requires careful attention to food intake; thus, neither the oral or subcutaneous route is suitable for the patient population typically affected by IIH, cluster headaches and related conditions. Such conditions are therefore undertreated in the population. Moreover, numerous other conditions and diseases that are treatable by agonists of one of more somatostatin receptors lack easily compliant delivery means, thus afflicted patient populations are unaddressed.

Acetazolamide and topiramate are carbonic anhydrase inhibitors that have been used to treat elevated intracranial pressure associated with IIH with limited success. However, side effects leading to significant dropout rates in clinical trials of these compounds have proven problematic. In the NORDIC trial (JAMA 2014; 311(16):1641-1651), the maximum tolerated dose of up to 4 g/day of acetazolamide improved vision, measured as perimetric mean deviation, papilledema grade and vision-related quality of life versus placebo; however, acetazolamide-treated patients experienced significant weight loss, adverse events that occurred in greater than 5% of study participants. Adverse events that occurred significantly more frequently in the acetazolamide group included paresthesia, dysgeusia, fatigue, decreased carbon dioxide level, nausea, vomiting, diarrhea, and tinnitus. Nine participants had adverse events that were classified as serious. Horton (JAMA 2015; 311(16): 1618-1619) noted the trial had a withdrawal rate of 19%, partly because patients with pseudotumor cerebri often face many challenges in life and have a propensity to miss appointments and drop out of treatment.

In another study on IIH, acetazolamide was compared to topiramate (Celebisoy et al., Acta Neurol. Scand. 2007: 116: 322-327). Topiramate was administered at doses ranging from 100 to 150 mg daily; acetazolamide at 1000 to 1500 mg/day. Significant improvement in visual field grades were reported in both groups. The most frequent side effects in the acetazolamide group were fatigue and tingling of the hands and feet reported by all the patients. The most common side effects in the topiramate group were distal paresthesias, concentration difficulties and weight loss ranging from 6 to 28 kg (mean: 9.75 kg) during 12 months period. This prominent weight loss recorded in the topiramate group during the study period was statistically significant when compared with the acetazolamide group.

These and other adverse effects of carbonic anhydrase inhibitors limit their usefulness. The prescribing information includes side effects including electrolyte disturbances, metallic taste and paresthesias for acetazolamide; fatigue, difficulties in concentration, manifest with problems with word finding problems, vomiting, diarrhea, foul or, decreased libido and weight loss for topiramate. Up to 20% of all patients treated with acetazolamide discontinued treatment due to adverse events.

A safe and effective treatment for IIH and cluster headache is needed that can reduce morbidity in the afflicted population and facilitate long-term compliance.

### SUMMARY

In one aspect, a method is provided for treating idiopathic intracranial hypertension (IIH) or cluster headache in a subject in need thereof, comprising administering to the subject a therapeutically effective combination of: (a) an intranasal dosing regimen of a somatostatin mimetic formulated for direct nose-to-brain administration, wherein the dosing regimen of the somatostatin mimetic provides a dose level or exposure that is less than the therapeutically effective dosing regimen when administered alone by a non-nasal route of administration; and (b) a dosing regimen of a carbonic anhydrase inhibitor, wherein the dosing regimen of the carbonic anhydrase inhibitor provides a dose level or exposure that is less than the therapeutically effective dose when administered alone.

In some embodiments, the dosing regimen of the carbonic anhydrase inhibitor elicits fewer or no side effects than that elicited by the therapeutically effective dose when administered alone. In some embodiments, a serum area-under-the-curve from a unit dose of the intranasal amount of the somatostatin mimetic is less than the serum area-under-the-curve of an effective unit dose of the somatostatin mimetic administered by a non-intranasal route. In some embodiments, a serum area-under-the-curve from a unit dose of the intranasal amount of the somatostatin mimetic is less than the serum area-under-the-curve from an effective unit dose of the somatostatin mimetic administered by the intranasal route for an indication other than IIH or cluster headache. In some embodiments, the indication other than IIH or cluster headache is acromegaly or carcinoid syndrome. In some embodiments, the non-intranasal route is subcutaneous, intramuscular, oral or intravenous.

In some embodiments, the effective intranasal amount of the somatostatin mimetic is about equal to or less than about 100 mcg daily. In some embodiments, the serum area-under-the-curve of the intranasal amount of the somatostatin mimetic is about equal to or less than about 50% of the serum area-under-the-curve of the somatostatin mimetic administered by a non-intranasal route. In some embodiments, the serum area-under-the-curve of the intranasal amount of the somatostatin mimetic is about equal to or less than about 25% of the serum area-under-the-curve of the somatostatin mimetic administered by a non-intranasal route. In some embodiments, the serum area-under-the-curve of the intranasal amount of the somatostatin mimetic is about equal to or less than about 10% of the serum area-under-the-curve of the somatostatin mimetic administered by a non-intranasal route. In some embodiments, the serum area-under-the-curve of the intranasal amount of the somatostatin mimetic is about equal to or less than about 50% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 25% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 10% of the serum area-under-the-curve of the somatostatin mimetic administered by the intranasal route for an indication other than IIH or cluster headache.

In some embodiments, the effective intranasal amount of the somatostatin mimetic is administered as a single daily dose, twice a day doses, three times a day doses, or four time a day doses, or as 2, 3 or 4 divided doses. In some embodiments, the effective intranasal amount is administered at a dose less frequently than daily, e.g., every other day, every third day, twice a week, or once weekly.

In some embodiments, the somatostatin mimetic is selected from somatostatin, octreotide, lanreotide, pasireotide, pentetreotide, or any combination thereof. In some embodiments, the somatostatin mimetic formulated for intranasal administration comprises a powder, liquid or gel. In some embodiments, the effective intranasal amount of somatostatin mimetic provides a minimal effective dose.

In some embodiments, the carbonic anhydrase inhibitor is administered orally. In some embodiments, the carbonic anhydrase inhibitor is administered daily.

In some embodiments, the carbonic anhydrase inhibitor is selected from among topiramate, acetazolamide, methazolamide, zonisamide, sulthiame, bichlorphenamide, or a combination thereof.

In some embodiments, the dose level, dose frequency, or combination thereof of a carbonic anhydrase inhibitor administered for the purposes disclosed herein, when administered alone, elicits fewer side effects than elicited by the dose level, dose frequency or combination thereof that is effective to treat approved indications.

In some embodiments, the dose level, dose frequency, or combination thereof of a carbonic anhydrase inhibitor administered for the purposes disclosed herein, when administered alone, elicits fewer side effects than elicited by the dose level, dose frequency or combination thereof that is effective to treat IIH or cluster headache.

In some embodiments, carbonic anhydrase inhibitor is acetazolamide and the dosing regimen is equal to or less than about 500 mg twice a day. In some embodiments, the dosing regimen is about 250 mg twice a day. In some embodiments, the dosing regimen is about 125 mg twice a day. In some embodiments, carbonic anhydrase inhibitor is acetazolamide and the dosing regimen is equal to or less than about 500 mg once a day. In some embodiments, the dosing regimen is about 250 mg once a day. In some embodiments, the dosing regimen is about 125 mg once a day.

In some embodiments, the carbonic anhydrase inhibitor is topiramate and the dosing regimen is equal to or less than about 50 mg twice a day. In some embodiments, the dosing regimen of topiramate is about 25 mg twice a day. In some embodiments, the dosing regimen of topiramate is about 15 mg twice a day. In some embodiments, the carbonic anhydrase inhibitor is topiramate and the dosing regimen is equal to or less than about 50 mg once a day. In some embodiments, the dosing regimen of topiramate is about 25 mg once a day. In some embodiments, the dosing regimen of topiramate is about 15 mg once a day.

In some embodiments, treating by the aforementioned method is to reduce or prevent an acute, ongoing episode, and/or prophylactic and/or maintenance therapy to prevent future episodes. In some embodiments, the methods disclosed herein provide for a therapeutically effective synergistic combination. In some embodiments, the therapeutically effective combination is a synergistic combination. In some embodiments, the effective therapeutic combination is administered daily, or more frequently, for a duration effective to treat or resolve the IIH or cluster headache.

In one aspect, a therapeutic combination for treatment of idiopathic intracranial hypertension (IIH) or cluster headache is provided, the therapeutic combination comprising: (a) one or more intranasal unit doses of a somatostatin mimetic formulated for direct nose-to-brain administration, wherein the dosing regimen of the somatostatin mimetic unit dose provides a dose level or exposure that is less than the therapeutically effective dosing regimen when administered alone by a non-nasal route of administration; (b) one or more unit doses of a carbonic anhydrase inhibitor, wherein the dosing regimen of a unit dose level of a carbonic anhydrase inhibitor provides a dose or exposure that is less than the therapeutically effective dose when administered alone; and (c) instructions for individual administration of each of the somatostatin mimetic and a carbonic anhydrase inhibitor in accordance with an effective dosing regimen.

In some embodiments, the intranasal dosing regimen of the somatostatin mimetic is direct nose-to-brain administration. In some embodiments, the dosing regimen of a carbonic anhydrase inhibitor elicits fewer or no side effects than that elicited by the therapeutically effective dose when administered alone.

In some embodiments, a serum area-under-the-curve of a unit dose of the somatostatin mimetic administered intranasally is less than the serum area-under-the-curve of the unit dose of the somatostatin mimetic administered by a non-intranasal route. In some embodiments, the non-intranasal route is subcutaneous, intramuscular oral or intravenous.

In some embodiments, a unit dose of the somatostatin mimetic formulated for intranasal administration is about equal to or less than about 100 mcg. In some embodiments, a unit dose of the somatostatin mimetic formulated for intranasal administration is about equal to or less than about 50 mcg. In some embodiments, a unit dose of the somatostatin mimetic formulated for intranasal administration is about equal to or less than about 10 mcg. In some embodiments, a unit dose of the somatostatin mimetic formulated for intranasal administration is about equal to or less than about 5 mcg. In some embodiments, a unit dose of the somatostatin mimetic formulated for intranasal administration is equal to about 1 mcg.

In some embodiments, the serum area-under-the-curve of a unit dose of the somatostatin mimetic administered intranasally is about equal to or less than about 50% of the serum area-under-the-curve of a unit dose of the somatostatin mimetic administered by a non-intranasal route. In some embodiments, the serum area-under-the-curve of a unit dose of the somatostatin mimetic administered intranasally is about equal to or less than about 25% of the serum area-under-the-curve of a unit dose of the somatostatin mimetic administered by a non-intranasal route. In some embodiments, the serum area-under-the-curve of a unit dose of the somatostatin mimetic administered intranasally is about equal to or less than about 10% of the serum area-under-the-curve of a unit dose of the somatostatin mimetic administered by a non-intranasal route. In some embodiments, a serum area-under-the-curve from a unit dose of the intranasal amount of the somatostatin mimetic is less than the serum area-under-the-curve from an effective single dose of the somatostatin mimetic administered by the intranasal route for an indication other than IIH or cluster headache. In some embodiments, the indication other than IIH or cluster headache is acromegaly or carcinoid syndrome.

In some embodiments, the unit dose of the somatostatin mimetic is labeled for administration in a single daily dose, twice a day dosing, three times a day dosing, four times a day dosing, or as 2, 3 or 4 divided doses.

In some embodiments, the somatostatin mimetic is selected from somatostatin, octreotide, lanreotide, pasireotide, pentetreotide, or any combination thereof. In some embodiments, the somatostatin mimetic formulated for intranasal administration comprises a powder, liquid or gel.

In some embodiments, the unit dose of somatostatin mimetic is a minimal effective dose.

In some embodiments, the carbonic anhydrase inhibitor is administered orally. In some embodiments, the carbonic anhydrase inhibitor is administered once daily or twice daily. In some embodiments, the dose level, dose frequency, or combination thereof of a carbonic anhydrase inhibitor administered for the purposes disclosed herein, is lower than effective to treat approved indications when administered alone.

In some embodiments, the carbonic anhydrase inhibitor is selected from among topiramate, acetazolamide, methazolamide, zonisamide, sulthiame, bichlorphenamide, or a combination thereof.

In some embodiments, the carbonic anhydrase inhibitor is acetazolamide and the dosing regimen is less than about 500 mg once a day or twice a day. In some embodiments, the dosing regimen is about 125 mg once a day or twice a day.

In some embodiments, the carbonic anhydrase inhibitor is topiramate and the dosing regimen is less than about 50 mg once a day or twice a day. In some embodiments, the carbonic anhydrase inhibitor is topiramate and the dosing regimen is about 25 mg once a day or twice a day. In some embodiments, the carbonic anhydrase inhibitor is topiramate and the dosing regimen is about 15 mg once a day or twice a day.

In some embodiments, the somatostatin mimetic of any of the foregoing embodiments is octreotide, which is provided as a liquid pharmaceutical composition comprising:
octreotide 0.1 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5

In some embodiment, the microcrystalline cellulose/sodium carboxymethycellulose comprises microcrystalline cellulose with about 11.3 to 18.8% sodium carboxymethylcellulose.

In some embodiments, the somatostatin mimetic of any of the foregoing embodiments is octreotide, which is provided as a liquid pharmaceutical composition comprising:
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

In some embodiments, the somatostatin mimetic of any of the foregoing embodiments is octreotide, which is provided as a liquid pharmaceutical composition consisting essentially of:
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

In some embodiments of the foregoing octreotide formulations, the compositions further comprise a tonicity adjusting agent such as mannitol, dextrose or sodium chloride, such that the tonicity is about 290 to about 500 mOsm/kg.

In some embodiments, use of the foregoing octreotide liquid pharmaceutical compositions is provided for direct nose-to-brain administration in a subject to treat idiopathic intracranial hypertension (IIH) or cluster headache. In some embodiments, the dose of octreotide is equal to or less than about 300 mcg per day, equal to or less than about 250 mcg per day, equal to or less than about 200 mcg per day, equal to or less than about 150 mcg per day, equal to or less than about 100 mcg per day, equal to or less than about 90 mcg per day, equal to or less than about 80 mcg per day, equal to or less than about 70 mcg per day, equal to or less than about 60 mcg per day, equal to or less than about 50 mcg per day, equal to or less than about 40 mcg per day, equal to or less than about 30 mcg per day, equal to or less than about 20 mcg per day, equal to or less than about 10 mcg per day, equal to or less than about 5 mcg per day. or about 1 mcg per day.

In some embodiments using the foregoing octreotide liquid formulations, the carbonic anhydrase inhibitor is acetazolamide and the dosing regimen is equal to or less than about 500 mg once a day or twice a day. In some embodiments, the dosing regimen is about 125 mg once a day or twice a day.

In some embodiments using the foregoing octreotide liquid formulations, the carbonic anhydrase inhibitor is topiramate and the dosing regimen is equal to or less than about 50 mg once a day or twice a day. In some embodiments, the carbonic anhydrase inhibitor is topiramate and the dosing regimen is about 25 mg once a day or twice a day. In some embodiments, the carbonic anhydrase inhibitor is topiramate and the dosing regimen is about 15 mg once a day or twice a day.

In some embodiments, the somatostatin mimetic of any of the foregoing embodiments is octreotide, which is provided as a powder pharmaceutical composition comprising
Octreotide 0.1%, mannitol 95%, hydrogenated lecithin 5%; or
Octreotide 0.5%, mannitol 95%, hydrogenated lecithin 5%; or
Octreotide 0.1%, calcium carbonate 95%, mannitol 2.5%, L-leucine 2.5%; or
Octreotide 0.5%, calcium carbonate 95%, mannitol 2.5%, L-leucine 2.5%.

In some embodiments, the somatostatin mimetic of any of the foregoing embodiments is octreotide, which is provided as a powder pharmaceutical composition consisting essentially of:
Octreotide 0.1%, mannitol 95%, hydrogenated lecithin 5%; or
Octreotide 0.5%, mannitol 95%, hydrogenated lecithin 5%; or
Octreotide 0.1%, calcium carbonate 95%, mannitol 2.5%, L-leucine 2.5%; or
Octreotide 0.5%, calcium carbonate 95%, mannitol 2.5%, L-leucine 2.5%.

In some embodiments of the foregoing, the powder pharmaceutical composition is prepared by spray drying, optionally at a temperature of 100° C. or 120° C.

In some embodiments, use of the foregoing powder pharmaceutical compositions of octreotide is provided for direct nose-to-brain administration in a subject to treat idiopathic intracranial hypertension (IIH) or cluster headache. In some embodiments, the dose of octreotide is equal to or less than about 300 mcg per day, equal to or less than about 250 mcg per day, equal to or less than about 200 mcg per day, equal to or less than about 150 mcg per day, equal to or less than about 100 mcg per day, equal to or less than about 90 mcg per day, equal to or less than about 80 mcg per day, equal to or less than about 70 mcg per day, equal to or less than about 60 mcg per day, equal to or less than about 50 mcg per day, equal to or less than about 40 mcg per day, equal to or less than about 30 mcg per day, equal to or less than about 20 mcg per day, equal to or less than about 10 mcg per day, equal to or less than about 5 mcg per day. or about 1 mcg per day.

In some embodiments using the foregoing octreotide powder formulations, the carbonic anhydrase inhibitor is acetazolamide and the dosing regimen is equal to or less than about 500 mg once a day or twice a day. In some embodiments, the dosing regimen is about 125 mg once a day or twice a day.

In some embodiments using the foregoing octreotide powder formulations, the carbonic anhydrase inhibitor is topiramate and the dosing regimen is equal to or less than about 50 mg once a day or twice a day. In some embodiments, the carbonic anhydrase inhibitor is topiramate and the dosing regimen is about 25 mg once a day or twice a day. In some embodiments, the carbonic anhydrase inhibitor is topiramate and the dosing regimen is about 15 mg once a day or twice a day.

In some embodiments, the therapeutic combination is to reduce or prevent an acute, ongoing episode, and/or prophylactic and/or maintenance therapy to prevent future episodes.

In some embodiments, the therapeutic combination is a synergistic combination.

In some embodiments, the dose level, dose frequency, or combination thereof of a carbonic anhydrase inhibitor when administered alone in accordance with the instructions provided therewith elicits fewer side effects than elicited by the dose level, dose frequency or combination thereof that is effective to treat approved indications. In some embodiments, the dose level, dose frequency, or combination thereof of a carbonic anhydrase inhibitor when administered alone in accordance with the instructions provided therewith elicits fewer side effects than elicited by the dose level, dose frequency or combination thereof that is effective to treat IIH or cluster headache.

In one aspect, a therapeutic combination for treatment of idiopathic intracranial hypertension (IIH) or cluster headache is provided, comprising: (a) one or more unit doses of a intranasal somatostatin mimetic formulated for direct nose-to-brain administration; (b) one or more unit doses of a carbonic anhydrase inhibitor; and (c) instructions for individual administration of each of the somatostatin mimetic and a carbonic anhydrase inhibitor in accordance with an effective dosing regimen; wherein the unit dose of a carbonic anhydrase inhibitor administered in accordance with the instructions elicits fewer side effects than the unit dose of a carbonic anhydrase inhibitor as administered alone for treatment of approved indications.

In one aspect, a therapeutic combination for treatment of idiopathic intracranial hypertension (IIH) or cluster headache is provided, comprising: (a) one or more unit doses of a intranasal somatostatin mimetic formulated for formulated for direct nose-to-brain administration; (b) one or more unit doses of a carbonic anhydrase inhibitor; and (c) instructions for individual administration of each of the somatostatin mimetic and a carbonic anhydrase inhibitor in accordance with an effective dosing regimen; wherein the unit dose of a carbonic anhydrase inhibitor administered in accordance with the instructions elicits fewer side effects than the unit dose of a carbonic anhydrase inhibitor as administered alone for treatment of idiopathic intracranial hypertension or cluster headache.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The terms “non-human animals” and “non-human mammals” are used interchangeably herein and include all vertebrates, e g, mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present disclosure and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

### Therapeutic Combinations

As disclosed herein, methods are provided for treating idiopathic intracranial hypertension (IIH) or cluster headache in a subject by administering to the subject a therapeutically effective combination. The therapeutic combination comprises:
(a) an intranasal dosing regimen of a somatostatin mimetic formulated for nose-to-brain administration, wherein its dosing regimen provides a dose level or exposure that is less than the therapeutically effective dosing regimen when administered alone by a non-nasal route of administration; and
(b) a dosing regimen of a carbonic anhydrase inhibitor, wherein its dosing regimen provides a dose level or exposure that is less than the therapeutically effective dose when administered alone.

The therapeutic combination and methods disclosed herein provides each of the components of the combination administered by a different route of administration. The somatostatin mimetic is administered intranasally. The carbonic anhydrase inhibitor is typically administered orally, though dosage forms for other routes of administration are available.

In some embodiments, the therapeutic combination is provided to effectively treat IIH or cluster headache in patients in which intranasal or direct nose-to-brain administration of a somatostatin mimetic alone is insufficiently effective. In some embodiments, the therapeutic combination is provided to effectively treat IIH or cluster headache in patients in which administration of a carbonic anhydrase inhibitor alone has an unacceptable side effect profile and may limit patient compliance. In some embodiments, the therapeutic combination is provided to effectively treat IIH or cluster headache in patients in which have not found benefit by any prior therapeutic regimen. In some embodiments, the therapeutic combination is provided to effectively treat IIH or cluster headache in patients who have failed treatment by another therapeutic regimen. In some embodiments, the therapeutic combination is provided to effectively treat IIH or cluster headache in patients in whom another therapeutic regimen has become less effective or ineffective.

In some embodiments, the therapeutic combination is provided to effectively treat IIH or cluster headache in a greater number or proportion of patients than the number or proportion of IIH or cluster headache patients in whom intranasal administration of a somatostatin mimetic alone is effective. In some embodiments, the therapeutic combination is provided to effectively treat IIH or cluster headache in patients and reduce the number or proportion of patients in which administration of a carbonic anhydrase inhibitor alone is poorly effective, ineffective and/or has side effect profile that may limit patient compliance.

As will be described in further detail below, the somatostatin mimetic is administered at a dose level and/or dosing frequency (referred to herein as dosing regimen) that is lower than is therapeutically effective, when administered alone by a non-nasal route. As will be described in further detail below, the carbonic anhydrase inhibitor dosing regimen is lower than is therapeutically effective when administered alone, or lower than the recommended prescribed dosage, wherein the therapeutically effective dose or recommended prescribed dosage is the dosing regimen used to treat any approved indication, such as but not limited to migraine, epilepsy monotherapy, epilepsy adjunctive therapy, glaucoma or mountain (altitude) sickness. In one embodiment, the therapeutically effective dose is the dosing regimen of the carbonic anhydrase inhibitor used alone to treat IIH.

As will also be described in further detail below, each component is administered at a dosing regimen that provides a lower systemic exposure than is therapeutically effective, in the case of the somatostatin mimetic, when administered alone by a non-nasal route, and in the case of a carbonic anhydrase inhibitor, a lower systemic exposure than therapeutically effective when administered alone. In some embodiments, therapeutically effective means effective for treating IIH or cluster headache. In some embodiments, therapeutically effective means effective for treatment of an approved indication.

In some embodiments, the dosing regimen of the carbonic anhydrase inhibitor that provides a dose level or exposure that is less than the therapeutically effective dose when administered alone, results in an improved side effect profile and/or reduces any one or more of the side effects that are reported on its product label.

In some embodiments, delivery of the intranasal formulation is direct “nose-to-brain” delivery wherein the formulation is contacted with the olfactory area or region within the nasal cavity, enabling transport of compounds directly into the brain via olfactory neurons.

Thus, therapeutic combination refers to the concurrent administration of the somatostatin mimetic and the carbonic anhydrase inhibitor by routes and dosage regimens specific for each, and not necessarily in the same dosage form. By way of non-limiting example, for which other examples are described herein below, the somatostatin mimetic is administered intranasally once a day, and a carbonic anhydrase inhibitor is administered orally twice a day. By way of non-limiting example, the somatostatin mimetic is administered intranasally twice a day, and the carbonic anhydrase inhibitor is administered orally twice a day. In one embodiment, where the dosing schedules of each component are the same, for convenience, both are administered at the same time (i.e., one right before or after the other). In other embodiments, the administration of the carbonic anhydrase inhibitor may be less frequent, on a daily basis, than the somatostatin mimetic. In other embodiments, the administration of the carbonic anhydrase inhibitor may be more frequent, on a daily basis, than the somatostatin mimetic.

In some embodiments, the methods and therapeutic combinations disclosed herein provide a synergistic effect, i.e., the therapeutic benefit of the combination is greater than the sum of the therapeutic benefit on IIH or cluster headache of each component administered in the absence of the other. Such synergy may be achieved, in certain embodiments, by titrating the dose level and dosing frequency (i.e., the dosing regimen) of each component of the combination individually. In some embodiments, the lower dose level or dose frequency of one or both component elicits fewer side effects. In some embodiments, the lower dose level or dose frequency of the carbonic anhydrase inhibitor elicits fewer side effects.

In some embodiments, the dose and dosing regimen of the somatostatin mimetic, in combination with the dose and dosing regimen of the carbonic anhydrase inhibitor, provide relief from symptoms such as headache and vision problems. In some embodiments wherein the condition or disease is acute or chronic, administration of the therapeutic combination is acute or chronic, and administration may also be prophylactic to prevent the future occurrence or recurrence of the condition or disease. These and other aspects of the methods, and therapeutic combinations are described in further detail below.

In other aspects, the methods and therapeutic combinations of a somatostatin mimetic and a carbonic anhydrase inhibitor described here are useful for treatment of a number of other conditions and diseases in which an agonist or ligand of a somatostatin receptor (any one or more of somatostatin receptor subtypes 1-5) is salutary.

### Conditions and Diseases Treated

In some embodiments, a therapeutic combination comprising a somatostatin mimetic and a carbonic anhydrase inhibitor may be used to treat acute or chronic idiopathic intracranial hypertension (IIH), sometimes referred to as pseudotumor cerebri, benign intracranial hypertension, primary or secondary IIH, cluster headache, pediatric intracranial hypertension, and related conditions, their symptoms and sequelae, by way of non-limiting examples. A subject may be treated as described herein for the first time after diagnosis of the condition or disease, or may be treated as described herein after failing one or more other therapies or one or more courses of one or more therapies. Furthermore, a subject may be treated to reduce or prevent an acute, ongoing episode, and/or treated prophylactically or with a maintenance dose therapy to prevent future episodes. Such rationales for starting and for continuation of dosing are full embraced herein.

As the nomenclature for these conditions and diseases has changed over time, some terms were or are synonyms for one or more others. This disclosure is not intended to exclude any such condition or disease because of changes in nomenclature or synonymy.

### Intracranial Hypertension

Intracranial Hypertension (IH) is characterized by increased pressure within the skull. Elevated cerebrospinal fluid (CSF) pressure presents with two symptoms, and can cause at least two problems: (1) headache, often severe; and (2) and visual changes. If the elevated CSF pressure remains untreated, permanent visual loss may occur.

There are no medications approved for IIH. No medication for IIH has been evaluated in a intranasal or ‘nose-to-brain’ intranasal delivery system, in combination with a carbonic anhydrase inhibitor

There are two categories of IIH: primary intracranial hypertension and secondary intracranial hypertension. Primary intracranial hypertension, now known as idiopathic intracranial hypertension (IIH), occurs without known cause. This form is known to occur in young, overweight, females in their reproductive years (ages 20-45). However, IIH can develop in both males and females of all ages and body types. Secondary intracranial hypertension has an identifiable cause including, but not limited, to drugs (such as tetracycline, lithium, vitamin A-derived oral acne medications or excessive ingestion of vitamin A, and oral or intrathecal steroids, growth hormone treatments, sleep apnea and certain systemic diseases such as lupus, leukemia, kidney failure (uremia), meningitis and dural venous sinus thrombosis. There is a known association of IIH and Chiari type I malformation. In patients diagnosed with IH, it is critical to rule out an intracranial space occupying lesions by neuro-imaging (CT or MRI).

Although many factors are known to trigger the disease, the mechanism by which IIH occurs, in either primary or secondary forms, is not known. In many cases, either type of IIH may be chronic.

The most common symptom of any form of IIH is often an unbearably painful or frequent headache, sometimes associated with nausea and vomiting, that is not relieved by any currently-available medication used off-label for this indication. The headache often awakens the patient from sleep and ICP is greatest in the morning hours. Some patients are treated in the emergency room where a lumbar puncture (spinal tap) is done to reduce ICP and to temporarily ease the headache. Measurement of the opening pressure is encouraged during these procedures in order to assess for intracranial hypertension.

The diagnosis is also confirmed by detecting a high spinal CSF pressure reading, usually greater than 250 mmH2O or 25 cmH2O (200-250 mmH2O or 20-25 cmH2O is considered borderline high) and normal laboratory and imaging studies including CT scan and/or MRI. There is generally a normal neurologic examination as well, although abnormal findings may be detected on eye examination. The eye findings may be subtle, and not noted in an emergency room evaluation. It is not uncommon to misdiagnose a patient with IIH as simply having a refractory migraine headache, and be treated as such. Unlike primary IH, secondary IIH patients may have abnormal scans and laboratory tests.

The high CSF pressure may cause the optic nerves to swell (papilledema). The optic nerve connects the interior of each eye, the retina, to the vision centers of the brain. The optic nerve transmits impulses from the retina to these brain centers. The earliest sign of papilledema on a visual field test is known as an enlarged blind spot. Abnormal CSF pressure can also affect the eye muscles controlling eye movements producing double vision, but this is an infrequent event. All patients with presumed IIH should have a thorough eye examination including visual field tests by an ophthalmologist or neuro-ophthalmologist.

Other common symptoms include transient altered vision, particularly on movement or bending over, intracranial noise (pulse synchronous tinnitus), stiff neck, back and arm pain, pain behind the eye, exercise intolerance, and memory difficulties.

Affected Populations. The number of patients diagnosed with and afflicted by IIH varies by territory in the general population and the annual incidence ranges from about 1-3 per 100,000. For obese women of child-bearing age, the incidence is about 22.5/100,000 and the prevalence is about 85.7/100,000 IIH occurs in men and children as well, but at a lower frequency. Weight is not usually a factor in men and in children under 10 years of age.

The true incidence of secondary IIH remains unknown because of the wide range of underlying causes and the lack of published surveys on the subject. Current statistics are not available on how many people have secondary intracranial hypertension.

Pediatric intracranial hypertension. This is the same disease; however, in the younger patients, obesity is not as common as it is in patients older than 14 years of age.

Related Disorders. Symptoms of the following disorders can be similar to those of IIH, and the pharmaceutical compositions and methods described here are also useful for their treatment.

Arachnoiditis is a progressive inflammatory disorder affecting the middle membrane surrounding the spinal cord and brain (arachnoid membrane). It may affect both the brain and the spinal cord and may be caused by foreign solutions (such as dye) being injected into the spine or arachnoid membrane. Symptoms may include severe headaches, vision disturbances, dizziness, nausea and/or vomiting. If the spine is involved, pain, unusual sensations, weakness and paralysis can develop.

Epiduritis is characterized by inflammation of the tough, outer canvas-like covering surrounding the brain and spinal cord known as the dura mater. Symptoms of this disorder can be similar to IIH.

Meningitis is an inflammation of the membranes around the brain and the spinal cord that can be caused by a number of different infectious agents such as bacteria, viruses, or fungi, or it may occur in the presence of malignant tumors. Meningitis may develop suddenly or have a gradual onset. Symptoms may include fever, headache, a stiff neck, and vomiting. The patient may also be irritable, confused and go from drowsiness, to stupor to coma.

The signs and symptoms of intracranial hypertension and related conditions are not intended to be limiting in any way to the amenability of treatment following the guidance of this disclosure.

### Cluster Headache(s)

The term “cluster headache” refers to headaches that have a characteristic grouping of attacks. Cluster headaches can occur up to eight times per day during a cluster period, which may last 2 weeks to 3 months, or longer. The headaches may disappear completely (go into “remission”) for months or years, only to recur at a later date. A cluster headache typically awakens a person from sleep 1 to 2 hours after going to bed. These nocturnal attacks can be more severe than the daytime attacks.

Cluster headaches are an uncommon type of primary headaches (i.e., a headache that has no structural cause), affecting less than 1 in 1,000 people. Cluster headaches are a young person's disease, and the headaches typically start before age 30. Cluster headaches are more common in men, but more women are starting to be diagnosed with this disorder. The male-to-female gender headaches appear to be six times more common in men than women, especially men in their 20s or 30s.

No medication for cluster headaches has been evaluated in intranasal or ‘nose-to-brain’ intranasal delivery systems, in combination with a carbonic anhydrase inhibitor.

The methods and pharmaceutical compositions disclosed herein are useful for the treatment of any of the aforementioned conditions and diseases, and those benefitted by agonizing one or more somatostatin receptor subtypes.

### Components of Therapeutic Combinations

### Somatostatin and Somatostatin Mimetics

Somatostatin is a peptide hormone also known as growth hormone inhibiting hormone, that is produced by various cells in the body and has various physiological effects. Somatostatin mimetic as used herein refers to any natural form of somatostatin and any chemically related or unrelated molecule that has biological activity that mimics the biological activity of somatostatin. Somatostatin has a broad range of biological actions that include the regulation of neurotransmission and secretion and the inhibition of the release of growth hormone (GH), thyroid-stimulating hormone (TSH), gastrointestinal (GI) hormones, pancreatic enzymes and neuropeptides. It modulates the rate of gastric emptying, smooth muscle contraction, and intestinal blood flow. It also inhibits the proliferation of both normal and tumor cells. Two biological forms of somatostatin exist: somatostatin-14 and -28, which are derived from a 92-amino acid pro-somatostatin precursor. Somatostatin and its analogs bind to receptors belonging to the seven transmembrane G protein coupled receptor superfamily Native somatostatin-14 binds to somatostatin receptor (SSTR) 1-4 with higher affinity, while somatostatin-28 is more SSTR5 selective.

The following provides a description of non-limiting examples of somatostatin mimetics useful for the purposed disclosed herein.

Somatostatin. The structure of somatostatin is shown below.

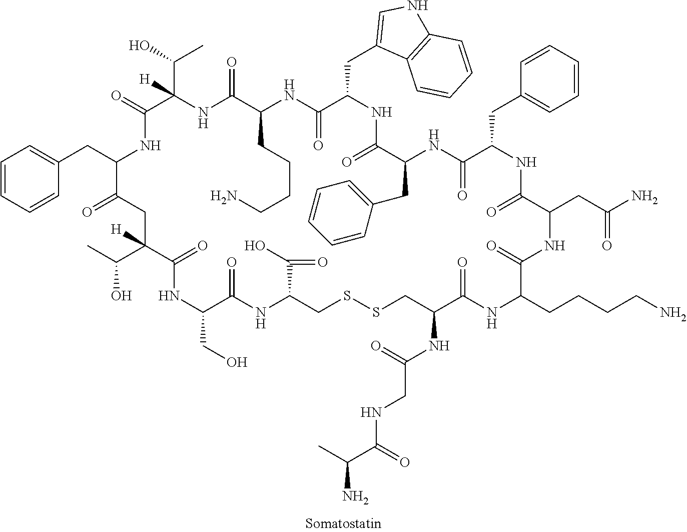 [SMILES: C[C@H](N)C(=O)NCC(=O)N[C@H]1CSSC[C@@H](C(=O)O)NC(=O)[C@H](CO)NC(=O)[C@H]([C@@H](C)O)CC(=O)C(Cc2ccccc2)NC(=O)[C@H]([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)C(CC(N)=O)NC(=O)C(CCCCN)NC1=O]

The IUPAC name is L-alanyl-glycyl-L-cysteinyl-L-lysyl-L-asparagyl-L-phenylalanyl-L-phenylalanyl-L-tryptophyl-L-lysyl-L-threonyl-L-phenylalanyl-L-threonyl-L-seryl-L-cysteine (3->14)-disulfide, and the IUPAC condensed nomenclature is H-Ala-Gly-Cys(1)-Lys-Asn-Phe-Phe-Trp-Lys-Thr-Phe-Thr-Ser-Cys(1)-OH, or in single-letter code, AGCKNFFWKTFTSC (wherein the C are disulfide linked). The molecular weight is 1637.9 g/mol. The chemical name is (4R,7S,10S,13S,16S,19S,22S,25S,28S,31S,34S,37R)-19,34-bis(4-aminobutyl)-31-(2-amino-2-oxoethyl)-37-[[2-[[(2S)-2-aminopropanoyl]amino]acetyl]amino]-13,25,28-tribenzyl-10,16-bis [(1R)-1-hydroxyethyl]-7-(hydroxymethyl)-22-(1H-indol-3-ylmethyl)-6,9,12,15,18,21,24,27,30,33,36-undecaoxo-1,2-dithia-5,8,11,14,17,20,23,26,29,32,35-undecazacyclooctatriacontane-4-carboxylic acid.

Somatostatin has two active forms produced by the alternative cleavage of a single preproprotein: one consisting of 14 amino acids (described above), the other consisting of 28 amino acids (H-Ser-Ala-Asn-Ser-Asn-Pro-Ala-Met-Ala-Pro-Arg-Glu-Arg-Lys-Ala-Gly-Cys-Lys-Asn-Phe-Phe-Trp-Lys-Thr-Phe-Thr-Ser-Cys-OH, disulfide bridge between Cys17 and Cys28). Having multiple synonyms including growth hormone-inhibiting hormone (GHIH), growth hormone release-inhibiting hormone (GHRIH), somatotropin release-inhibiting factor (SRIF), and somatotropin release-inhibiting hormone (SRIH), somatostatin is produced by delta cells of the digestive system, acts directly on the acid-producing parietal cells via a G-protein coupled receptor (which inhibits adenylate cyclase, thus effectively antagonizing the stimulatory effect of histamine) to reduce acid secretion. Somatostatin can also indirectly decrease stomach acid production by preventing the release of other hormones, including gastrin and histamine which effectively slows down the digestive process.

More relevant to the present disclosure, somatostatin is produced by neuroendocrine neurons of the ventromedial nucleus of the hypothalamus. These neurons project to the median eminence, where somatostatin is released from neurosecretory nerve endings into the hypothalamo-hypophysial system through neuron axons. Somatostatin is then carried to the anterior pituitary gland, where it inhibits the secretion of growth hormone from somatotrope cells. The somatostatin neurons in the periventricular nucleus mediate negative feedback effects of growth hormone on its own release; the somatostatin neurons respond to high circulating concentrations of growth hormone and somatomedins by increasing the release of somatostatin, so reducing the rate of secretion of growth hormone. In the brain, somatostatin inhibits the release of growth hormone (GH), inhibits release of thyroid-stimulating hormone (TSH), inhibits adenyl cyclase in parietal cells, and inhibits release or prolactin (PRL).

Somatostatin (and its mimetics; non-limiting examples described elsewhere herein) are useful therapeutically to treat a number of conditions and diseases such as but not limited to acromegaly, pituitary and neuroendocrine tumors, VIPomas and carcinoid syndrome.

The biological half-life of somatostatin is 2-3 minutes, thus for therapeutic use, mimetics of somatostatin have been developed with longer biological half-lives. The disclosure herein embraces all such mimetics of somatostatin, whether modifications of the somatostatin peptide, or unrelated molecules with the biological activity of somatostatin.

Octreotide. Octreotide (e.g., SANDOSTATIN) is an octapeptide analogue of somatostatin with the chemical structure is shown below.

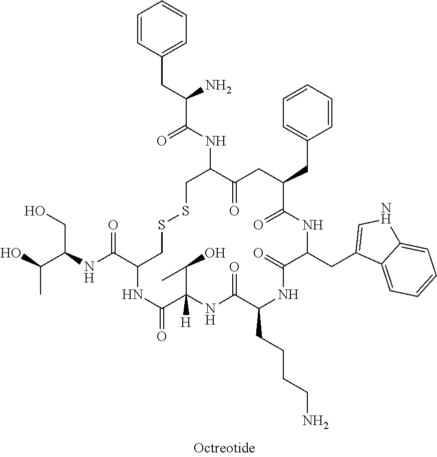 [SMILES: C[C@@H](O)[C@@H]1NC(=O)[C@H](CCCCN)NC(=O)C(Cc2c[nH]c3ccccc23)NC(=O)[C@H](Cc2ccccc2)CC(=O)C(NC(=O)[C@H](N)Cc2ccccc2)CSSCC(C(=O)N[C@H](CO)[C@@H](C)O)NC1=O]

Octreotide mimics the biological activity of somatostatin, and is used for the treatment of growth hormone producing tumors (causing acromegaly and gigantism) when surgery is contraindicated, and treatment of pituitary tumors that secrete thyroid-stimulating hormone. Octreotide is marketed as an acetate salt.

Octreotide is available in a vial for injection (SANDOSTATIN Injection) in a buffered lactic acid solution for administration by deep subcutaneous (intrafat) or intravenous administration. It is available in 1 mL ampuls in three strengths: containing 50, 100 or 500 mcg octreotide (as acetate), and 5 mL multi-dose vials in two strengths, containing 200 and 1000 mcg/mL octreotide (as acetate). For treatment of acromegaly, dosage may be initiated at 50 mcg three times a day. The most common dose is 100 mcg three times a day but some patients required up to 500 mcg three times a day for maximum effectiveness. For carcinoid tumors, 100-600 mcg per day is suggested; in clinical studies the median daily maintenance dose was 450 mcg; some patients benefitted from as little as 50 mcg and some required up to 1500 mcg/day. For VIPomas, daily dosages of 200-300 mcg in 2-4 divided doses are recommended during the initial 2 weeks of therapy. For ongoing therapy, doses above 450 mcg/day are not usually required.

Octreotide is also available as an intramuscular (gluteal) formulation for depot administration (SANDOSTATIN LAR DEPOT). It is indicated for treatment in patients who have responded to and tolerated SANDOSTATIN Injection subcutaneous injection for: (1) Acromegaly, (2) Severe diarrhea/flushing episodes associated with metastatic carcinoid tumors and (3) Profuse watery diarrhea associated with Vasoactive Intestinal Peptide (VIP) secreting tumors (1.3). SANDOSTATIN LAR DEPOT is an injectable suspension in vials of 10 mg octreotide per 6 mL, 20 mg per 6 mL or 30 mg per 6 mL. The dosage for patients not currently receiving SANDOSTATIN injection subcutaneously is (1) Acromegaly: 50 mcg three times daily SANDOSTATIN Injection subcutaneously for 2 weeks followed by SANDOSTATIN LAR DEPOT 20 mg intragluteally every 4 weeks for 3 months; (2) Carcinoid Tumors and VIPomas: SANDOSTATIN Injection subcutaneously 100-600 mcg/day in 2-4 divided doses for 2 weeks followed by SANDOSTATIN LAR DEPOT 20 mg every 4 weeks for 2 months. For patients currently receiving SANDOSTATIN injection subcutaneously: (1) Acromegaly: 20 mg every 4 weeks for 3 months; (2) Carcinoid Tumors and VIPomas: 20 mg every 4 weeks for 2 months. For renal impairment, patients on dialysis: 10 mg every 4 weeks. For hepatic impairment, patients with cirrhosis: 10 mg every 4 weeks.

Octreotide is also available as a subcutaneous formulation for administration in a prefilled pen injector type device (BYNFEZIA) indicated for (1) reduction of growth hormone (GH) and insulin-like growth factor 1 (IGF-1) [somatomedin C] in adult patients with acromegaly who have had inadequate response to or cannot be treated with surgical resection, pituitary irradiation, and bromocriptine mesylate at maximally tolerated doses; (2) treatment of severe diarrhea/flushing episodes associated with metastatic carcinoid tumors in adult patients; and (3) treatment of profuse watery diarrhea associated with vasoactive intestinal peptide tumors (VIPomas) in adult patients. The device contains 2.8 mL of 2,500 mcg/mL octreotide. The doses are: (1) acromegaly: initiate dosage at 50 mcg three times daily. Typical dosage is 100 mcg three times a day; (2) carcinoid Tumors: 100-600 mcg daily in 2-4 divided doses for first 2 weeks; (3) VIPomas: 200-300 mcg daily in 2-4 divided doses for first 2 weeks. Octreotide (acetate) is also available in a delayed-release capsule for oral use called MYCAPSSA. For efficacy, administration of oral octreotide must not occur in proximity of a meal (e.g., at least one hour before or two hours after). Such dose timing restriction may not coincide with the need for treatment or a patient's schedule, for those indications described herein.

Lanreotide. Lanreotide (SOMATULINE DEPOT) is a synthetic polypeptide analogue of somatostatin that resembles the native hormone in its ability to suppress levels and activity of growth hormone, insulin, glucagon and many other gastrointestinal peptides.

Lanreotide acetate is a synthetic cyclical octapeptide analog of the natural hormone, somatostatin. Lanreotide acetate is chemically known as [cyclo S-S]-3-(2-naphthyl)-D-al anyl-L-cys teinyl-L-tyro syl-D-tryptophyl-L-lys yl-L-valyl-L-cys teinyl-L-threoninamide, acetate salt. Its molecular weight is 1096.34 (base) and its amino acid sequence is:

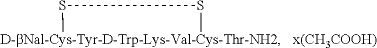

where x=1.0 to 2.0. Because its half-life is longer than somatostatin, lanreotide can be used clinically to treat neuroendocrine tumors that secrete excessive amounts of growth hormone (acromegaly) or other active hormones or neuropeptides. Lanreotide has many side effects including suppression of gall bladder contractility and bile production, and maintenance therapy may cause cholelithiasis and pancreatitis as well accompanying liver injury. SOMATULINE DEPOT is a somatostatin analog indicated for: (1) the long-term treatment of acromegalic patients who have had an inadequate response to or cannot be treated with surgery and/or radiotherapy; (2) the treatment of adult patients with unresectable, well- or moderately-differentiated, locally advanced or metastatic gastroenteropancreatic neuroendocrine tumors (GEP-NETs) to improve progression-free survival; (3) the treatment of adults with carcinoid syndrome; when used, it reduces the frequency of short-acting somatostatin analog rescue therapy. It is for deep subcutaneous injection only, in the superior external quadrant of the buttock. Recommended dosages are: (1) Acromegaly: 90 mg every 4 weeks for 3 months. Adjust thereafter based on GH and/or IGF-1 levels; (2) GEP-NETs: 120 mg every 4 weeks; (3) carcinoid Syndrome: 120 mg every 4 weeks.

Pasireotide. Pasireotide (SIGNIFOR and SIGNIFOR LAR) is a synthetic polypeptide analogue of somatostatin that resembles the native hormone in its ability to suppress levels and activity of growth hormone, insulin, glucagon and many other gastrointestinal peptides. The structure is:

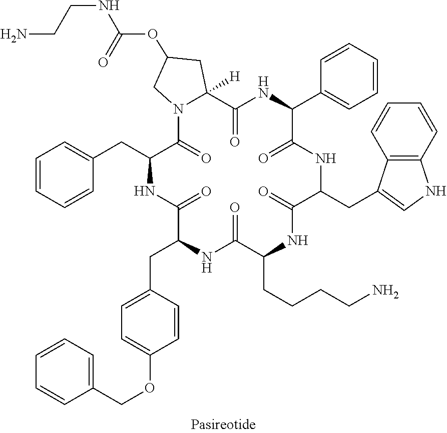 [SMILES: NCCCC[C@@H]1NC(=O)C(Cc2c[nH]c3ccccc23)NC(=O)[C@H](c2ccccc2)NC(=O)[C@@H]2CC(OC(=O)NCCN)CN2C(=O)[C@H](Cc2ccccc2)NC(=O)[C@H](Cc2ccc(OCc3ccccc3)cc2)NC1=O]

Because its half-life is longer than somatostatin, pasireotide can be used clinically to treat neuroendocrine pituitary tumors that secrete excessive amounts of growth hormone causing acromegaly, or adrenocorticotropic hormone (ACTH) causing Cushing disease. Pasireotide has many side effects including suppression of gall bladder contractility and bile production, and maintenance therapy can cause cholelithiasis and accompanying elevations in serum enzymes and bilirubin.

The foregoing descriptions of various somatostatin mimetics is intended to be illustrative and non-limiting with regard to the somatostatin mimetics useful for the purposes described herein.

### Intranasal Formulations and Delivery Devices

Any of the foregoing, non-limiting examples of a somatostatin analogue may be used in an intranasal formulation and delivery system for the treatment of conditions and diseases such as and including those described herein. In one aspect, intranasal delivery is via direct (also called deep) nose-to-brain delivery, i.e., to the olfactory region of the nasal cavity, in one embodiment, to directly access the blood brain barrier.

Direct “nose-to-brain” delivery of a somatostatin mimetic may be achieved by use of any one of several methods that comprise a nasal formulation and/or a nasal delivery device to deliver the somatostatin mimetic to the roof of the nasal cavity, where transport into the central nervous system (CNS) is achieved. Nose-to-brain delivery is a minimally invasive drug administration pathway, which bypasses the blood-brain barrier as the drug is directed from the nasal cavity to the brain.

In one aspect, intranasal delivery is via nose-to-brain delivery, i.e., to the olfactory area or region of the nasal cavity. As will be seen in the description below, the efficacious dose of a somatostatin mimetic for the conditions and diseases described herein and in general those benefitted by an intranasally delivered somatostatin mimetic, is substantially lower than the dose required by a non-intranasal route, when administered in a therapeutic combination with a carbonic anhydrase inhibitor. Moreover, the efficacious dose of a somatostatin mimetic for the conditions and diseases described herein and in general those benefitted by the direct nose-to-brain delivery of a somatostatin mimetic, is substantially lower than the dose required by a non-intranasal route, when administered in a therapeutic combination with a carbonic anhydrase inhibitor. In other embodiments, the efficacious dose of a somatostatin mimetic for the conditions and diseases described herein and in general those benefitted by the direct nose-to-brain delivery of a somatostatin mimetic, is substantially lower than the dose required by a non-intranasal route, when administered in a therapeutic combination with a carbonic anhydrase inhibitor at a lower dose than used to treat approved indications. Moreover, the efficacious dose of a somatostatin mimetic for the conditions and diseases described herein and in general those benefitted by the direct nose-to-brain delivery of a somatostatin mimetic, is substantially lower than the dose required by a nasal route of administration that is not nose-to-brain administration, when administered in a therapeutic combination with a carbonic anhydrase inhibitor at a lower dose than used to treat approved indications.

A somatostatin mimetic may be formulated in, and administered by, a formulation and/or nasal delivery device wherein the somatostatin mimetic is delivered into the nasal cavity, e.g., to the olfactory region of the nasal cavity, enters the brain circulation and treats a condition or disease such as and including those described herein. Intranasal formulations and delivery devices provide such administration, and direct (or deep) nose-to-brain delivery further enhances the administration and further reduces the efficacious dose. See, for example, Wang et al., 2019, “Nose-to-Brain Delivery, Journal of Pharmacology and Experimental Therapeutics September 2019, 370 (3) 593-601. As disclosed herein, various means of achieving delivery of peptides and other molecules such as somatostatin mimetic into the brain circulation may comprise the use of uptake enhancers and other components, to achieve absorption via the nose-to-brain route, using powder, liquid or gel formulations. The disclosure herein is not limited to any particular method or formulation for achieving delivery of the somatostatin mimetic intranasally, e.g., direct nose-to-brain routes. A preferred method is by direct nose-to-brain delivery. As will be described further below, in some embodiments, the amount of an intranasally delivered somatostatin mimetic necessary to achieve the treatment of a condition of disease described herein is substantially reduced compared to the amount of the somatostatin mimetic needed for efficacious treatment delivered by a non-intranasal route such as intravenous, intramuscular, oral or subcutaneous, or substantially reduced compared to an intranasal delivery method that is not directed to nose-to-brain. In some aspects, the efficacious amount of somatostatin mimetic for treating IIH and cluster headache is substantially reduced compared with the amount needed to treat a different condition or disease, such as acromegaly, by the intranasal route. While not wishing to be bound by theory, treatment of IIH and cluster headache using a somatostatin mimetic delivered into the brain by the intranasal route, in combination with a carbonic anhydrase inhibitor, may be achieved with a substantially lower amount of somatostatin mimetic than heretofore administered by other routes for treatment of the other conditions and diseases described herein, and a substantially lower amount than heretofore administered intranasally for treatment of conditions and disease not among those described herein, such as targets outside of the central nervous system. As will be described below, the dose (amount per nasal administration) and dosing regimen (e.g., frequency of intranasal administration of the dose per day, duration of dosing in days or longer, among others) factor into achieving an efficacious, brain exposure of a somatostatin mimetic delivered intranasally for the treatment of IIH, cluster headaches, among other conditions and diseases. The dose of carbonic anhydrase inhibitor may also be substantially lower than the dose needed alone to treat such a condition.

Examples of formulations useful for achieving nose-to-brain delivery of a somatostatin mimetic are described herein, but are not intended to be limiting, and may be used in combination with a carbonic anhydrase inhibitor delivered by its usual route of delivery, e.g., orally. Non-limiting examples of agents useful for preparing intranasal formulations herein include bioadhesives polymers such as those described by Ilium et al., The nasal delivery of peptides and proteins, T Biotech, 9, 284-289, 1991, incorporated herein by reference. Non-limiting examples of bioadhesives polymers include mucoadhesive polymers. Non-limiting examples of mucoadhesive polymers include chitosan, amylose, amylopectin, carbopol, cellulose, carboxymethylcellulose, sodium alginate, gellan gum, hyaluronan and poloxamer. Such polymers may be used singly or in any combination in a formulation disclosed herein. Use of chitosan is described in Ritthdej, 2011, Nasal delivery of peptides and proteins with chitosan and related mucoadhesive polymers, Peptide and Protein Delivery, Chapter 3, 47-68, incorporated herein by reference.

The present disclosure provides for somatostatin mimetic formulations that comprise one or more agents that facilitate or enable nose-to-brain delivery. Such agents are provided to overcome barriers to absorption include those that prevent degradation, enhance barrier permeability by transient opening of tight junctions, disrupting lipid bilayer packing/complexation/carrier/ion pairing and enhancing resident time/slowing down Mucociliary activity, as non-limiting examples.

In one embodiment, somatostatin mimetic formulations disclosed herein comprise one or more permeation enhancers. By way of non-limiting examples, such compounds are classified as surfactants, cyclodextrins, protease inhibitors, cationic polymers and tight junction modulators may be formulated with a somatostatin mimetic to facilitate nose-to-brain delivery. One or more of such types of compounds may be included in a formulation as disclosed herein. It is recognized that both specific and general formulation components and agents described herein may fall into more than one type of category; thus, one or more of any such compounds regardless of categorization may be a component of a formulation herein.

Surfactants. Surface active agents, or surfactants, are amphiphilic molecules possessing both lipophilic and hydrophilic residues. Surfactants have various applications in nasal drug administration, due to their high interfacial activity; one of which is as an absorption enhancer. Surfactants can enhance absorption with more than one mechanism; these include perturbing the cell membrane by leaching of membrane proteins, opening of tight junctions, or preventing enzymatic degradation of the drugs. Surfactants used as absorption enhancers can be classified as phospholipids, bile salts, (such as sodium taurocholate etc.), non-ionic surfactants (e.g. sorbitan ester such as monolaurate and monostearate, polysorbates such as polysorbate 20 and polysorbate 80), salt of fatty acids and alkyl glycosides (e.g., tetradecylmaltoside, N-lauryl-β-D-maltopyranoside etc.).

Cyclodextrins. Cyclodextrins are a family of cyclic oligosaccharides consisting of a macrocyclic ring of glucose subunits joined by α-1,4 glycosidic bonds. Cyclodextrins are composed of 5 or more α-D-glucopyranoside units linked 1->4, as in amylose (a fragment of starch). Typical cyclodextrins contain a number of glucose monomers ranging from six to eight units in a ring, creating a cone shape: a (alpha)-cyclodextrin: 6 glucose subunits, 13 (beta)-cyclodextrin: 7 glucose subunits, y (gamma)-cyclodextrin: 8 glucose subunits. The inclusion compounds of cyclodextrins with hydrophobic molecules are able to penetrate body tissues including mucosal penetration of drugs. A cyclodextrin may be included in a formulation disclosed herein.

In some embodiments, a permeation enhancer used in a formulation as disclosed herein is a calcium-binding agent.

Cationic polymers. Polymeric systems with positive charges or modified with cationic entities, incorporated on their backbone and/or side chains, are called cationic polymers. These polymers are able to enhance absorption of macromolecules. Cationated gelatins, cationated pullulans, poly-L-arginine, polyethyleneimine, chitosan and its derivatives, an derivatives of any of the foregoing, are non-limiting examples of cationic polymers useful for the purposes disclosed herein. Cationic polymers interact with the mucosal barriers and enhance the absorption of water-soluble macromolecules via tight junction modification. Chitosan is a bioadhesive, cationic polysaccharide that are able to transiently modulate the paracellular permeability of intestinal and nasal epithelia enhancing the absorption of small and macromolecular compounds. Other tight junction modulators effective on tight junction proteins include claudins (e.g., CLDN1, CLDN2, CLDN3, CLDN4, CLDN5, CLDN6, CLDN7, CLDN8, CLDN9, CLDN10, CLDN11, CLDN12, CLDN13, CLDN14, CLDN15, CLDN16, CLDN17, CLDN18, CLDN19, CLDN20, CLDN21, CLDN22, CLDN23) and zonnula occludans (ZO) proteins (e.g., ZO-1, ZO-2, ZO-3).

Mucoadhesives. As described above, non-limiting examples of mucoadhesive polymers include chitosan, amylose, amylopectin, carbopol, cellulose, dextran, carboxymethylcellulose, sodium alginate, gellan gum, hyaluronan and poloxamer. Such polymers may be used singly or in any combination in a formulation disclosed herein.

Buffers. Non-limiting examples of aqueous buffers useful in a somatostatin mimetic formulation disclosed herein include citrate, TRIS, HEPES, acetate, phosphate, phosphate-buffered saline, MOPS and MES. A formulation disclosed herein may comprise one or more buffering agents.

Preservatives. Formulations described herein may further comprise one or more preservatives to inhibit microbial activity and maintain stability of the somatostatin mimetic, including anti-oxidants. Non-limiting examples of preservatives useful herein include benzalkonium chloride (BAC), thimerosal, benzyl alcohol, butylated hydroxytoluene, butylated hydroxyanisole, chlorobutanol, EDTA, menthol, methylparaben, propylparaben, potassium sorbate,

Viscosity modifiers. Non-limiting examples of viscosity modifiers useful in a somatostatin mimetic formulation disclosed herein include hydroxypropylmethyl cellulose (hypromellose; HPMC), microcrystalline cellulose (MCC), MCC/carboxymethylcellulose mixtures, dextran and chitosan, and any combination thereof.

Tonicity modifiers. Non-limiting examples of tonicity modifiers useful in a somatostatin mimetic formulation disclosed herein include mannitol, dextrose, sodium chloride, sorbitol and maltitol, and any combination thereof.

Other component may be included in formulations disclosed herein, such as an aerosol flowability enhancer or dispersant, e.g., silicon dioxide, L-leucine, dileucine (Leu-Leu), trileucine (Leu-Leu-Leu), Leu-His-Leu, and Lys-Gly-Asp-Ser, by way of non-limiting examples.

Non-limiting examples of other components in a formulation disclosed herein include lactose, water-absorbing, water-insoluble, water-swellable or water-soluble polymers such as polyacrylates such as sodium polyacrylate, potassium polyacrylate and ammonium polyacrylates; lower alkyl ethers of cellulose such as methyl cellulose, hydroxyethyl cellulose, hydroxypropyl cellulose and sodium carboxymethyl cellulose; polyvinyl pyrrolidone, amylose, polyethylene glycol e.g. of MW from 400 to 8000, hydroxypropylmethyl cellulose, microcrystalline cellulose, cellulose, α-cellulose, and cross-linked sodium carboxymethyl cellulose, water-absorbing and water-insoluble starches such as hydroxypropyl starch, carboxymethyl starch, water-absorbing and water-insoluble proteins such as gelatin, casein; water-absorbing and water-insoluble gums such as gum arabic, tragacanth gum and glucomannan; and cross-linked vinyl polymers such as cross-linked polyvinyl pyrrolidone, cross-linked carboxyvinyl polymer or its salt, cross-linked polyvinyl alcohol and polyhydroxyethylmethylacrylate. Solvents including water, ethyl alcohol, propylene glycol, etc. may be used in preparing formulations including those to be spray dried or otherwise prepared as powder formulations.

Thus, in some embodiments, a liquid or powder formulation for nose-to-brain delivery of a somatostatin mimetic such as but not limited to octreotide may comprise any one of more of the following components: a permeation enhancer, and/or a mucoadhesive, and/or a protease inhibitor, and/or a buffer, and/or a preservative.

For powder formulations, such formulation may comprise any one or more of the foregoing components, which are typically lyophilized, spray dried, agglomerated, milled, any combination thereof, or otherwise formed into a powder from a liquid formulation. Such powder formulations may comprise a carrier such as but not limited to calcium carbonate, mannitol, lecithin, lactose, sorbitol, maltitol, or any combination thereof.

For gel formulations, such formulations may comprise thixotropic components such as but not limited to hypromellose, pectin, gellan gum, and sodium hyaluronate. Such gelling agents are provided to remain as a gel at rest but produce fine droplets on shear thinning when a pump is actuated. Gel formulations comprising octreotide for nose-to-brain delivery are embraced herein.

The aforementioned components may be provided in liquid, powder or gel formulations for achieving nose-to-brain delivery of a somatostatin mimetic. Typically, the formulation is used with a delivery device to deliver the formulation into the nose, e.g., into or onto the olfactory region in the nasal cavity. Non-limiting examples of such devices are described herein below. In some instances, specific devices are used for specific formulations, as specific devices or formulations were developed based on using specific formulations or devices, but the requirement for a specific device is not so limiting provided it delivery the desired formulation and achieves effective nose-to-brain delivery. The ensuing descriptions provide exemplary liquid and powder formulations as guidance in achieving the purposes disclosed herein.

For any of the formulations disclosed herein, the amount or unit dose of somatostatin mimetic e.g. octreotide, delivered per administration may be from less than about 500 mcg to about 1 mcg. In some formulations, the amount of somatostatin mimetic, e.g., octreotide, is 300 mcg per unit dose, equal to or less than about 250 mcg per unit dose, equal to or less than about 200 mcg per unit dose, equal to or less than about 150 mcg per unit dose, equal to or less than about 100 mcg per unit dose, equal to or less than about 90 mcg per unit dose, equal to or less than about 80 mcg per unit dose, equal to or less than about 70 mcg per unit dose, equal to or less than about 60 mcg per unit dose, equal to or less than about 50 mcg per unit dose, equal to or less than about 40 mcg per unit dose, equal to or less than about 30 mcg per unit dose, equal to or less than about 20 mcg per unit dose, equal to or less than about 10 mcg per unit dose, equal to or less than about 5 mcg per unit dose, or equal to or greater than about 1 mcg per unit dose.

Thus, for example, for a delivered dose of 10 micrograms of octreotide using a device that delivers 10 mg per pump, the concentration of octreotide in the formulation would be 0.1%. For a delivered dose of 10 micrograms of octreotide using a device that delivers 50 mg per pump, the concentration of octreotide in the formulation would be 0.02%. For a delivered dose of 80 micrograms of octreotide using a device that delivers 80 mg per pump, the concentration of octreotide in the formulation would be 0.1%. For a delivered dose of 80 micrograms of octreotide using a device that delivers 50 mg per pump, the concentration of octreotide in the formulation would be 0.16%. For a delivered dose of 50 micrograms of octreotide using a device that delivers 10 mg per pump, the concentration of octreotide in the formulation would be 0.5%. For a delivered dose of 100 micrograms of octreotide using a device that delivers 20 mg per pump, the concentration of octreotide in the formulation would be 0.5%.

### Liquid Formulations

Any of the methods, therapeutic combinations and unit doses disclosed herein may utilize a liquid formulation of the somatostatin mimetic, which in some embodiments is octreotide. Any of the ensuing descriptions of liquid formulations may be provided for the somatostatin mimetic, e.g., octreotide, in any method, therapeutic combination, unit dose, or any other embodiment herein.

Excipients useful for preparing liquid formulations disclosed herein may include but are not limited to polyethylene glycol 400 (PEG400), mannitol, Avicel (e.g., Avicel CL611), lecithin and polysorbate 80 (TWEEN 80), in addition to those described elsewhere herein. Such excipients may be included singly or in any combination. Non-limiting examples of liquid formulations suitable for the purposes described herein include those set forth below. Abbreviations: BAC, benzalkonium chloride; PEG, polyethylene glycol. Percentages of octreotide are shown as free base.

In some embodiments, a liquid formulation comprises about 0.1 to about 1 mg/mL octreotide; about 0.5 to about 10% of a polymer such chitosan, amylose, amylopectin, carbopol, cellulose, carboxymethylcellulose, sodium alginate, gellan gum, hyaluronan or poloxamer, or any combination thereof; about 0.5 to about 10% of a surfactant such as a bile salt, phospholipid, alkylglycoside, polysorbate 80; and about 0.005 to about 0.1% of a preservative. In some embodiments, the formulation is provided in a buffer such as citrate, TRIS, HEPES, acetate, phosphate, phosphate-buffered saline, MOPS or MES. In some embodiments the pH is about 4-6.

In some embodiments, a liquid formulation comprises about 0.1 to about 1 mg/mL octreotide, about 0.5 to about 10% (v/v) microcrystalline cellulose/sodium carboxymethycellulose (e.g., Avicel CL611), about 0.5 to about 10% polysorbate 80 (w/v), and about 0.005 to about 0.1% BAC (w/v). In some embodiments, the formulation is provided in a citrate buffer, e.g., about 0.025 M to about 0.05 M citrate, and a pH of about 4-6.

In some embodiments, the liquid formulation consists essentially of about 0.1 to about 1 mg/mL octreotide, about 0.5 to about 10% (v/v) microcrystalline cellulose/sodium carboxymethylcellulose (e.g., Avicel CL611), about 0.5 to about 10% polysorbate 80 (w/v), and about 0.005 to about 0.1% BAC (w/v). In some embodiments, the formulation further consists of a citrate buffer, e.g., about 0.025 M to about 0.05 M, and a pH of about 4-6.

In some embodiments, the formulation comprises octreotide 0.1 mg/mL, Avicel CL611 2% (w/v), polysorbate 80 2% (w/v), BAC 0.02% (w/v) in 0.01M citrate buffer pH 4.5. In some embodiments the formulation comprises octreotide 1.0 mg/mL, Avicel CL611 2% (w/v), polysorbate 80 2% (w/v), BAC 0.02% (w/v) in 0.01M citrate buffer pH 4.5.

In some embodiments, the formulation consists essentially of octreotide 0.1 mg/mL, Avicel CL611 2% (w/v), polysorbate 80 2% (w/v), BAC 0.02% (w/v) in 0.01M citrate buffer pH 4.5. In some embodiments the formulation consists essentially of octreotide 1.0 mg/mL, Avicel CL611 2% (w/v), polysorbate 80 2% (w/v), BAC 0.02% (w/v) in 0.01M citrate buffer pH 4.5.

In some embodiments, the octreotide formulation comprises Avicel CL611 2% (w/v), polysorbate 80 2% (w/v), BAC 0.02% (w/v) in 0.01M citrate buffer pH 4.5. In some embodiments the formulation comprises octreotide 1.0 mg/mL, Avicel CL611 2% (w/v), polysorbate 80 2% (w/v), BAC 0.02% (w/v) in 0.01M citrate buffer pH 4.5.

In some embodiments, the octreotide formulation consists essentially of octreotide and Avicel CL611 2% (w/v), polysorbate 80 2% (w/v), BAC 0.02% (w/v) in 0.01M citrate buffer pH 4.5. In some embodiments the formulation consists essentially of octreotide 1.0 mg/mL, Avicel CL611 2% (w/v), polysorbate 80 2% (w/v), BAC 0.02% (w/v) in 0.01M citrate buffer pH 4.5.

In some embodiments, the liquid formulation comprises about 0.1 to about 1 mg/mL octreotide, about 0.5 to about 10% (v/v) polyethylene glycol 400, about 0.5 to about 10% polysorbate 80 (w/v), and about 0.005 to about 0.1% BAC (w/v). In some embodiments, the formulation is provided in a citrate buffer, e.g., about 0.025 M to about 0.05 M, and a pH of about 4-6.

In some embodiments, the liquid formulation consists essentially of about 0.1 to about 1 mg/mL octreotide, about 0.5 to about 10% (v/v) polyethylene glycol 400, about 0.5 to about 10% polysorbate 80 (w/v), and about 0.005 to about 0.1% BAC (w/v). In some embodiments, the formulation further consists of a citrate buffer, e.g., about 0.025 M to about 0.05 M, and a pH of about 4-6.

In some embodiments, the formulation comprises octreotide 1.0 mg/mL, PEG 400 2% (w/v), polysorbate 80 2% (w/v), BAC 0.02% (w/v) in 0.01M citrate buffer pH 4.5. In some embodiments the formulation comprises octreotide 0.1 mg/mL, PEG 400 2% (w/v), polysorbate 80 2% (w/v), BAC 0.02% (w/v) in 0.01M citrate buffer pH 4.5.

In some embodiments, the formulation consists essentially of octreotide 1.0 mg/mL, PEG 400 2% (w/v), polysorbate 80 2% (w/v), BAC 0.02% (w/v) in 0.01M citrate buffer pH 4.5. In some embodiments the formulation consists essentially of octreotide 0.1 mg/mL, PEG 400 2% (w/v), polysorbate 80 2% (w/v), BAC 0.02% (w/v) in 0.01M citrate buffer pH 4.5.

In some embodiments, the octreotide formulation comprises PEG 400 2% (w/v), polysorbate 80 2% (w/v), BAC 0.02% (w/v) in 0.01M citrate buffer pH 4.5. In some embodiments the formulation comprises octreotide 0.1 mg/mL, PEG 400 2% (w/v), polysorbate 80 2% (w/v), BAC 0.02% (w/v) in 0.01M citrate buffer pH 4.5.

In some embodiments, the formulation consists essentially of octreotide and PEG 400 2% (w/v), polysorbate 80 2% (w/v), BAC 0.02% (w/v) in 0.01M citrate buffer pH 4.5. In some embodiments the formulation consists essentially of octreotide 0.1 mg/mL, PEG 400 2% (w/v), polysorbate 80 2% (w/v), BAC 0.02% (w/v) in 0.01M citrate buffer pH 4.5.

As noted above, depending on the amount of formulation delivered per pump of a delivery device used to administer the formulation and the desired dose per administration, the octreotide concentration in the formulation is correspondingly adjusted. The concentration may be, for example, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 mg/mL. In some embodiments, the concentration may be from about 1 to about 10 mg/ml. In some embodiments, the concentration may be 1, 2, 3, 4, 5, 6, 7, 8, 9, or 10 mg/mL. In some embodiments, the amount of octreotide delivered per administration (e.g., using a delivery device or pump) is about 1, 5, 10, 20, 30, 40, 50, 60, 70, 80, 90, 100, 150, 200, 250, 300, 350, 400, 450 or 500 microgram (mcg). In some embodiments, the volume of liquid formulation delivered per administration (e.g., using a delivery device or pump) is about 10, 20, 30, 40, 50, 60, 70, 80, 90, 100, 110, 120, 130, 140, 150, 160, 170, 180, 190 or 200 microliters (mcl).

In some embodiments, the liquid formulation is administered once a day (q.d.). In some embodiments the liquid formulation is administered twice a day (b.i.d.). In some embodiments the liquid formulation is administered three times a day (t.i.d.). In some embodiments the liquid formulation is administered more than three times a day (e.g., four times a day, q.i.d.). The dosing frequency, duration, and other aspects of the dosing regimen may be guided by the health care professional to achieve a desired clinical effect.

Any of the foregoing liquid formulations or any other liquid formulations comprising a somatostatin mimetic as described herein, may be provided for delivery using any one of a number of liquid delivery devices, such as but not limited to the VP7-232NE device of Aptar Pharmaceuticals.

In some embodiments, the droplet size of a liquid formulation comprising a somatostatin mimetic disclosed herein may be less than about 120 μm. In some embodiments, the droplet size is between about 15 μm and about 120 μm, between about 15 μm and about 100 μm between about 15 μm and about 80 μm, between about 15 μm and about 70 μm, between about 30 μm and about 70 μm or between about 30 μm to about 40 μm.

In some embodiments, the spray content uniformity of a liquid somatostatin mimetic formulation disclosed herein may be within about 1% relative standard deviation (RSD). In some embodiments, the spray content uniformity of a liquid somatostatin mimetic formulation disclosed herein may be within about 100% of target delivered dose (TDD).

In some embodiments, the pH of a liquid somatostatin mimetic formulation disclosed herein have a pH of about 4.5, a pH of between about 4.0 and about 5.0, a pH of between about 4.0 and about 4.5, a pH of between about 4.5 and 5.0, or a pH of between about 4 and about 6. In some embodiments, the desired buffering pH is achieved using a buffer such as but not limited to citrate, TRIS, HEPES, acetate, phosphate, phosphate-buffered saline, MOPS or MES. The final pH of a formulation is adjusted using an acid or base as needed, such as HCl or NaOH.

In some embodiments, the osmolality of a liquid somatostatin mimetic formulation disclosed herein is between about 290 and 500 mOsm/kg. In some embodiments, the osmolality of the formulation as described herein in increased to within about 290 to about 500 mOsm/kg by the additional of a tonicity agent. Non-limiting examples of tonicity agents include mannitol, dextrose, sodium chloride, sorbitol, maltitol, fructose, or any combination thereof.

In some embodiments, the viscosity of a liquid somatostatin mimetic formulation disclosed herein, η (Pa s) is about 0.4 to about 1.05.

### Powder Formulations

Any of the methods, therapeutic combinations and unit doses disclosed herein may utilize a powder formulation of the somatostatin mimetic, which in some embodiments is octreotide. Any of the ensuing descriptions of powder formulations may be provided for the somatostatin mimetic, e.g., octreotide, in any method, therapeutic combination, unit dose, or any other embodiment herein.

A powder formulation of a somatostatin mimetic such as octreotide may be prepared using excipients such as mannitol, calcium carbonate, lactose, sorbitol, maltitol, L-leucine, lecithin (phosphatidylcholine; e.g. LIPOID S 80, soybean phospholipids with 75% phosphatidylcholine), hydrogenated phosphatidylcholine (e.g., PHOSPHOLIPON 80 H [hydrogenated soybean phospholipids, >70% phosphatidylcholine]), in addition to those described elsewhere herein. Such excipients may be included singly or in any combination or any combination thereof.

In some embodiments, a powder formulation comprises about 0.1 to about 1% octreotide; about 85 to about 99% of a carrier such as mannitol, dextrose, lactose, sorbitol, fructose, or calcium carbonate; and about 1% to about 10% of a phospholipid such as lecithin.

In some embodiments, a powder formulation comprises about 0.1 to about 1% octreotide, about 85 to about 99% calcium carbonate; about 1 to about 5% mannitol, dextrose, lactose, sorbitol, fructose, or any combination thereof; and about 1% to about 10% L-leucine.

In some embodiments, mannitol is provided at about 85 to about 99%. In some embodiments, mannitol is provided at 95%.

In some embodiments, the mannitol is provided at about 1 to about 10%. In some embodiments the mannitol is provided at about 2.5%.

In some embodiments the L-leucine is provide at about 1 to about 10%. In some embodiments, the L-leucine is provided at 2.5%.

In some embodiments the lecithin (e.g., phosphatidyl choline or hydrogenated phosphatidylcholine) is provided at about 1 to about 10%. In some embodiments, the hydrogenated lecithin is provided at about 2.5%.

In some embodiments, the calcium carbonate is provided at about 80% to about 99%. In some embodiments the calcium carbonate is 95%.

In some embodiments, a mannitol/lecithin formulation comprises 95% mannitol and 5% hydrogenated phosphatidylcholine.

In some embodiments, a calcium carbonate/mannitol/L-leucine formulation comprises 95% calcium carbonate, 2.5% mannitol and 2.5% L-leucine.

In powder formulations, the somatostatin mimetic such as octreotide is provided at about 0.1% to about 1.0%. In some embodiments, the octreotide is 0.1% or 0.5%. As noted above, depending on the amount of formulation delivered per pump of a delivery device used to administer the formulation and the desired dose per administration, the octreotide concentration in the formulation is correspondingly adjusted. The concentration may be, for example, about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1%. In some embodiments, the concentration may be from about 1 to about 10%, In some embodiments, the concentration may be 1, 2, 3, 4, 5, 6, 7, 8, 9, or 10%. In some embodiments, the amount of octreotide delivered per administration (e.g., using a delivery device) is about 1, 5, 10, 20, 30, 40, 50, 60, 70, 80, 90, 100, 150, 200, 250, 300, 350, 400, 450 or 500 microgram (mcg). In some embodiments, the amount of powder formulation delivered per administration (e.g., using a delivery device) is about 1, 5, 10, 20, 30, 40, 50, 60, 70, 80, 90, 100, 110, 120, 130, 140, 150, 160, 170, 180, 190 or 200 milligrams (mg). In some embodiments, the amount of powder formulation delivered per administration (e.g., using a delivery device) is about 1, 5, 10, 20, 30, 40, 50, 60, 70, 80, 90, 100, 110, 120, 130, 140, 150, 160, 170, 180, 190 or 200 mm3 (cubic mm).

In some embodiments, the powder formulation is administered once a day (q.d.). In some embodiments the powder formulation is administered twice a day (b.i.d.). In some embodiments the powder formulation is administered three times a day (t.i.d.). In some embodiments the powder formulation is administered more than three times a day (e.g., four times a day, q.i.d.). The dosing frequency, duration, and other aspects of the dosing regimen may be guided by the health care professional to achieve a desired clinical effect.

Non-limiting examples of powder formulations useful for the purposes disclosed herein include the following (in w/w of powder formulation). Each formulation was spray dried at either 100° C. or 120° C. to evaluate the resulting powder (see Examples).
Octreotide 0.1%, mannitol 95%, lecithin 5%.
Octreotide 0.5%, mannitol 95%, lecithin 5%.
Octreotide 0.1%, calcium carbonate 95%, mannitol 2.5%, L-leucine 2.5%
Octreotide 0.5%, calcium carbonate 95%, mannitol 2.5%, L-leucine 2.5%

Such powder formulation are prepared from a liquid formulation then made into a powder by methods such as lyophilization and spray drying. The material may be further milled, agglomerated, pulverized, sieved, etc. to achieve a powder characteristic compatible with a powder delivery device.

As will be seen in the examples below, liquid and powder formulations of octreotide were developed that will deliver the requisite dose by nose-to-brain delivery for the uses herein described. The European Pharmacopoeia HPLC method for Octreotide quantification was used to study liquid and powder formulations of octreotide for nose-to-brain delivery.

For the liquid formulation, results for the Droplet Size Distribution Dv (50) values at 3 cm, for Formulation 1 ranged from 33.25-35.88 μm, Formulation; Formulation 2 ranged from 32.98-37.5 μm, Formulation 3 ranged from 31.71-33.32 μm and Formulation 4 ranged from 31.92-32.98 μm. The Dv 50 values at 6 cm, for Formulation 1 ranged from 38.96-41.60 μm, Formulation 2 ranged from 38.16-41.93 μm, Formulation 3 ranged from 35.66-39.36 μm and Formulation 4 ranged from 37.29-41.97 μm. The Spray Content Uniformity was performed with each formulation with an n=10. Recovery for all Formulations were very close to the Target Delivered Doses of either 10 or 100 μg. pH measurements were very similar for all formulations and within the pH range for the nasal cavity. Osmolality results showed the Avicel containing formulations 1 and 2 had values of 31.33 and 32.67 mOsm, while the PEG 400 containing formulations 3 and 4 had values of 83.67 and 80.00 mOsm; which tonicities can be increased using any one of more of a number of agents. Viscosity testing were also completed with the Avicel containing formulations showed increased viscosity, where the PEG 400 containing formulations showed very little change in viscosity.

For the powder formulations, all 8 formulations have been successfully spray dried and generated suitable yields of recovery. All powders have been characterized using in-vitro tools. For TGA: all powders showed low/negligible levels of moisture. For SCU, Delivered Doses were consistent for all formulations.

As noted above, depending on the amount of formulation delivered per insufflation of a delivery device used to administer the formulation, the octreotide concentration in the formulation is correspondingly adjusted.

### Delivery Devices

Notwithstanding the disclosure herein of a liquid or powder formulation comprising a somatostatin mimetic such as octreotide, a formulation disclosed herein is typically used in combination with a delivery device to deliver the liquid, gel, or powder formulation into the nose and ideally to the olfactory region. In some embodiments, the methods and formulations disclosed herein are achieved and delivered using such a delivery device.

Not wishing to be bound by theory and for which no duty of disclosure is necessary, nasal spray drug products contain therapeutically active pharmaceutical ingredients (API; e.g. octreotide) dissolved or suspended in non-pressurized dispensers that deliver a spray containing a metered dose of the API. The dose can be metered by the spray pump or pre-metered during manufacture. Upon actuation of the dose from nasal spray systems, the spray events can be divided into three phases: (1) formation, (2) fully developed/stable, and (3) dissipation. In the formation phase, flow through the spray pump nozzle is relatively low, droplet size is large, and the output of the nasal spray product is not yet stable. Flow is also low during dissipation at the end of the spray event when the metering chamber is empty. Nasal solution or suspension drug products consist of API particles either dissolved or suspended in an aqueous system in the presence of a range of different excipients, respectively. These excipients can range from preservatives, viscosity modifiers, emulsifiers and buffering agents. For suspension nasal products, the API particle size is a key critical quality attribute, which will affect emitted API particle size and regional deposition of API in the nose. In addition, the particle size of the API will affect the rate of dissolution and permeability at site of deposition in the nasal epithelium and thereby systemic exposure of the API from the nose. Other physicochemical properties of the API such as polymorphic form, solvated/hydrated form, rugosity and surface properties may also affect the rate of API dissolution and therefore affect systemic exposure from the nose. In the case of peptide formulations careful characterization of the molecule is undertaken to ensure drug product quality requirements are met. For both solution and suspension nasal sprays droplet size and rheological properties of the formulation will impact the regional deposition. Such characterization is described herein in the Examples below, such that the formulations described herein achieve the desired features for efficacious nose-to-brain delivery for successfully treating the conditions disclosed herein.

In some embodiments, droplet sizes of 50 μm favored anterior deposition (mainly at the nasal vestibule), and particles of this size are unable to flow freely within the nasal perplexed anatomy. In some embodiments, droplets within the 2.6-14.3 μm particle size range and 10-11 μm diameter achieve maximum deposition in the central nasal areas. Rheological properties influence spray droplet sizes are known to impact on nasal regional deposition. Agents such as but not limited to pectin, gellan gum, and sodium hyaluronate have been used as in situ gelling system to develop a nasal suspension formulations. These agents have shown to increase turbinate deposition posterior to the nasal valve. Such gelling agents may create thixotropic formulation systems that remain gel like at rest but produce fine droplets on shear thinning when the pump is actuated. In some embodiments, thixotropic formulations containing hypromellose are able to achieve 84% turbinate deposition. While, in some embodiments, viscosity is confused with rheology of nasal formulations, when just the viscosity component of the formulation is increased there is generally decreased deposition into the turbinate and olfactory regions of the nose.

In one embodiment, Aptar Pharma manufactures the UDS (solution or powder formulation) single shot nasal unidose system. In the Examples herein, this system has been selected for the feasibility study based on its key features which make it designed to enable the systemic delivery of drugs without the need for injection or administration by a healthcare professional but has the ability to be administered by a second party. Primeless, with one handed actuation and 360° functionality. This system has a liquid dose up to 100 μL in volume or 140 mm3 powder dosage. In some embodiments, the UDS system is used with a liquid or powder formulation disclosed herein to achieve the purposes hereof.

As shown in the Examples herein, in developing a solution formulation of octreotide, the solubility of the compound under pH range of 4-8 is desired, in one embodiment, since this pH is well tolerated in the nasal cavity. In some embodiments, the addition of dextran, sorbitol, fructose, dextrose, mannitol or sodium chloride modulates the tonicity and viscosity of the formulation together with other buffering agents. In some embodiments, an osmolality target for the formulation is about 290 mOsm/kg to prevent damage to the nasal cilia. In some embodiments the osmolality of the formulation is between about 290 and about 500 mOsm/kg.

In one embodiment, a powder formulation system is prepared by co-spray drying a solution of octreotide in the presence of calcium carbonate, leucine and mannitol. In one embodiment, a powder formulation system is prepared by co-spray drying a solution of octreotide in the presence of mannitol and lecithin. In one embodiment, a powder formulation system is prepared by co-spray drying a solution of octreotide in the presence of trimethyl-chitosan, leucine and mannitol. In one embodiment, such construct creates a powder formulation with high dispersibility and contain absorption enhancer within the matrix. The density of powder should be between 0.6-0.8 g/mL which will enable a wide range of doses that could be filled into the UDS/P system.

Descriptions of some such systems are provided herein but are not intended in any way to be limiting as to the selection of the formulation and/or device used for intranasal delivery. Reference to patent and publications is intended merely as a guide. Each and every reference herein is incorporated herein by reference.

### Intranasal Formulations of Somatostatin Mimetics

A somatostatin mimetic such as but not limited to those described above may be provided for administration in any formulation compatible with intranasal administration including direct nose-to-brain administration. The intranasal formulation may be a powder, a liquid, a gel, or any other form that achieves the intranasal delivery of a somatostatin mimetic as described herein. IIH and cluster headache are among conditions and diseases treatable by agonists or ligands of one or more somatostatin receptor subtypes 1-5, and wherein intranasal administration offers a facile and patient-compliant means for dosing.

Alkylglycosides and related compounds can be used to enhance intranasal delivery of peptides. U.S. Pat. No. 8,833,728 (to Aegis Therapeutics LLC) describes a pharmaceutical compositions and methods for delivering a polypeptide to the central nervous system of a mammal via intranasal administration. Nasal absorption is enhanced using, for example, an aqueous composition comprising a therapeutic polypeptide and a compound such as 1-O-n-dodecyl-beta-D-maltopyranoside, 1-O-n-decyl-beta-D-maltopyranoside, 1-O-n-tetradecyl-beta-D-maltopyranoside, and beta-D-fructopyranosyl-alpha-glucopyranoside monododecanoate. In some embodiments, the compound is present in the aqueous composition at a concentration between 0.125 g to 1 g per 100 ml of the aqueous composition. U.S. patent Ser. No. 10/046,025 (also to Aegis Therapeutics LLC) describes an intranasal formulation comprising dodecyl-β-D-maltoside. Other patents include U.S. Pat. Nos. 7,998,927, 8,133,863, 8,226,949, EP EP18731691.4 and U.S. Ser. No. 16/615,362, all of which are incorporated herein by reference. Additional intranasal formulations and delivery systems are described in Wang et al, 2019, ibid, which is incorporated herein by reference in its entirety.

Formulations comprising a somatostatin mimetic in a liposomal particle including nanoparticles are embraced herein. Such formulations are described by Ong et al., Nose-to-brain drug delivery by nanoparticles in the treatment of neurological disorders, Curr Med Chem. 2014; 21(37):4247-56; Ghadiri et al., Strategies to Enhance Drug Absorption via Nasal and Pulmonary Routes, Pharmaceutics 2019, 11, 113; by way of non-limiting example.

The present disclosure also embraces somatostatin mimetic formulations used for routes of delivery other than intranasal, such as but not limited to intravenous, intramuscular, subcutaneous, and oral. In some embodiments, a sustained release or depot formulation of a somatostatin mimetic is utilized for intranasal administration as described here. Non-limiting examples of such formulations include microspheres described in U.S. Pat. Nos. 7,399,486, 9,877,922, and 9,155,702 (to Peptron Inc.); other formulations are described elsewhere herein.

Other examples of intranasal formulations are described for particular nasal delivery systems and devices, as described further below. Such intranasal formulations may be used together with the particular delivery system or device as described, or may be used with a different delivery device including devices such as a nasal spray, pumps, insufflators or atomizers.

### Intranasal Delivery Devices

Nose-to-brain delivery devices, and their respective formulations of a somatostatin mimetic, are useful to achieve the purposes disclosed herein. A somatostatin mimetic such as but not limited to those described above may be provided for administration using any device compatible with intranasal administration, and on one aspect, for nose-to-brain delivery.

U.S. patent Ser. No. 10/507,295 (to Impel NeuroPharma Inc) describes a device for delivery of a compound to an olfactory region of a nasal cavity, the device comprising: an actuator body comprising a vertical portion and an angled portion, the vertical portion comprising a contoured surface to accommodate a facial component of a user's face and configured to house a pressurized propellant container containing propellant and a first portion of a connection channel, the first portion of the connection channel configured to be in communication with the propellant container, the angled portion comprising an additional contoured surface to accommodate an additional component of the user's face and configured to house a second portion of the connection channel; a diffuser in communication with the second portion of the connection channel; a drug chamber in communication with the diffuser, the drug chamber configured to hold the compound; a nozzle in communication with the drug chamber, wherein propellant released from the propellant container is configured to travel through the connection channel, contact the diffuser, and propel the compound out the nozzle forming a plume.

U.S. Pat. No. 8,978,647 (to OptiNose AS) describes a nasal delivery device for delivering substance to a nasal cavity of a subject, the delivery device comprising: a nosepiece for fitting to a nostril of a subject, wherein the nosepiece comprises a tip element which includes a delivery aperture from which substance is in use delivered into the nasal cavity, and the tip element is at least in part tapered so as to be inclined to a longitudinal axis of the nosepiece, with the delivery aperture extending both laterally across the tip element and along a longitudinal extent of the tip element in relation to the longitudinal axis, wherein the tip element of the nosepiece has differing lengths adjacent forwardmost and rearwardmost sections thereof; a nozzle through which substance is in use delivered to the respective nasal cavity; and a delivery unit for delivering substance through the nozzle of the nosepiece.

U.S. Pat. No. 7,841,337 (to OptiNose AS) describes an Exhalation Delivery System (EDS) actuated by breath, wherein a mouthpiece through which a user in use exhales to actuate the delivery device; a nosepiece for fitting to a nostril of the user through which a substance is in use delivered; a substance supply unit actuatable to deliver a dose of a substance through the nosepiece; a loading unit operable to load the substance supply unit with an actuation force; and a release mechanism for enabling actuation of the substance supply unit in response to exhalation by the user through the mouthpiece; wherein the release mechanism comprises a locking unit which is movable between a locking configuration in which the substance supply unit is locked in a non-actuated position when loaded by the loading unit and a release configuration in which the substance supply unit is actuatable by the loading unit, and a trigger member for releasing the locking unit from the locking configuration to the release configuration in response to exhalation by the user through the mouthpiece and thereby enabling actuation of the substance supply unit.

U.S. Pat. No. 10,238,577 (to Zeteo Biomedical LLC) describes devices for delivery of fluid compositions include a delivery device including an internally pierced blister in which the device includes a plunger configured to crush the blister and deliver the contents in a lateral direction with respect to the motion of a dispensing button. Such devices can be used for intranasal delivery. International patent publication WO2020198536 (to Zeteo Biomedical, LLC) describes a handheld assembly for dispensing a medicament to a subject. The assembly includes a unit dose device, a shell, a plunger, a dispense button, a drive member and an escapement that is movable and capable of cycling the dispense assembly thru multiple states of a dispense cycle. Intranasal delivery including nose-to-brain delivery are described.

Another example of an intranasal delivery system comprises pressurized compartment containing medicament and excipient, pre-loaded into a lightweight casing, wherein finger-tip snap compression of the casing results in cyclonic motion for accurate drug release of powdered formulations; see U.S. Pat. Nos. 7,163,013 and 8,695,592 (to Alchemy Pharmatech Ltd).

Any of the devices and formulations described herein may be used for the purposes disclosed herein. Other formulations and devices to achieve effective intranasal delivery are described below. All of the citations herein are incorporated by reference.

In some embodiments, the intranasal formulation and delivery system comprises an exhalation delivery system such as described in U.S. Pat. Nos. 10,076,614, 10,076,615, 10,124,132, and 10,179,216, incorporated herein by reference in their entireties.

In some embodiments, the intranasal formulation and delivery system comprises a delivery system as described in any one or more of U.S. Pat. Nos. 10,765,829; 10,737,045; 10,722,667; 10,695,295; 10,682,414; 10,653,745; 10,639,438; 10,639,437; 10,549,052; 10,525,218; 10,478,574; 10,478,405; 10,456,377; 10,420,750; 10,406,200; 10,398,859; 10,300,229; 10,286,164; 10,252,010; 10,220,097; 10,179,216; 10,144,776; 10,124,132; 10,112,021; 10,099,024; 10,099,019; 10,076,615; 10,076,614; 10,066,017; 9,962,397; 9,949,989; 9,949,923; 9,855,247; 9,833,504; 9,744,210; 9,682,205; 9,649,456; 9,629,894; 9,566,402; 9,556,260; 9,522,243; 9,468,727; 9,452,272; 9,339,617; 9,320,800; 9,308,234; 9,308,191; 9,272,104; 9,249,424; and US patent publication 20190269867, all of which are incorporated by reference herein.

In some embodiments, the intranasal formulation and delivery system comprises a delivery system as described in any one or more of U.S. Pat. Nos. 10,653,690; 10,617,682; 10,549,052; 10,292,948; 10,213,487; 10,085,937; 9,775,838; 9,707,226; 9,629,965; 9,561,177; 9,480,644; 9,468,747; 9,211,253; 9,033,939; 8,567,390; all of which are incorporated by reference herein.

The foregoing are merely non-limiting examples of nasal formulations and nasal delivery systems that can be used to deliver the somatostatin mimetic for the purposes disclosed herein.

### Unit Dose of Somatostatin Mimetic

In reference to the somatostatin mimetic of the methods and therapeutic combinations disclosed herein, unit dose refers to the amount of somatostatin mimetic (dose amount) delivered into the nose per single prescribed time of administration (e.g., one insufflation in each nostril, two insufflations in each nostril, etc.). As noted herein, the unit dose of the somatostatin mimetic in the intranasal formulation is provided such that the intranasal formulation is effective in the treatment of any condition or disease described herein, and moreover, is effective at a dose level and dosing regimen (e.g., dose level, frequency of daily administration, duration of dosing in days or longer time periods, the time of start and ending of a course of a dosing regimen and cycles of a dosing regimen, among others) that is lower than the dose and dosing regimen of the same somatostatin mimetic administered by a route other than intranasal for the treatment of the conditions and diseases described herein, or lower than the dose and dosing regimen of the same somatostatin mimetic administered intranasally for the treatment of conditions and diseases other than described herein. The unit dose may be prescribed for administration one time or multiple times per day, e.g., one insufflation in each nostril, three times a day; two insufflations in each nostril twice a day, two insufflations in each nostril every other day, etc.). Dosing regimen refers to the dose administration factors such as frequency of daily dosing (e.g., once [q.d.], twice [b.i.d], thrice [t.i.d], four times [q.i.d], five times and so forth, including every other day, every third day, twice a week, weekly, and so forth); duration of administration (e.g., daily for one or more days, one week or more than one week, one month or more than one month, chronically, and any combination or components of any of the foregoing); cycles of dosing with periods of no dosing, such as one week of dosing with a one week no-dosing period, and repeated; two week cycles with a week or two weeks of non-dosing in between, etc.). In some instances, dosing regimen may include the dose (dose level). Such dosing regimens may be provided for treating an acute episode as well as for providing maintenance therapy to prevent or reduce recurrence. In some embodiments, the maintenance dose will be of a lower dose level, a reduced dosing frequency, or the combination of both, to reduce or protect from recurrence.

The intranasal formulation and/or delivery device may comprise a single unit dose packaging of the somatostatin mimetic, or may be provided in multiple unit dose packaging. Such format of packaging will be provided for facile storage of the formulation and/or device, and provide for facile administration depending on the prescribed dosing regimen. A combination package comprising the somatostatin mimetic formulation and/or delivery device, and the carbonic anhydrase inhibitor dosage form, are described herein below.

In some embodiments, the dose and dosing regimen of the somatostatin mimetic described herein provides for an effective administration that is lower (e.g., lower total daily dose) than the subcutaneous dose and dosing regimen of the same somatostatin mimetic. In some embodiments, the dose and dosing regimen described herein provides for an effective administration that is lower (e.g., lower total daily dose) than the intravenous dose and dosing regimen of the same somatostatin mimetic. In some embodiments, the dose and dosing regimen described herein provides for an effective administration that is lower (e.g., lower total daily dose) than the intramuscular dose and dosing regimen of the same somatostatin mimetic. In some embodiments, the dose and dosing regimen described herein provides for an effective administration that is lower (e.g., lower total daily dose) than the deep subcutaneous dose and dosing regimen of the same somatostatin mimetic. In some embodiments, the dose and dosing regimen described herein provides for an effective administration that is lower (e.g., lower total daily dose) than the depot dose and dosing regimen of the same somatostatin mimetic. In some embodiments, the dose and dosing regimen described herein provides for an effective administration that is lower (e.g., lower total daily dose) than the oral dose and dosing regimen of the same somatostatin mimetic. In any of the foregoing embodiments, the lower dose and dosing regimen is for treating the same condition or disease.

Moreover, the efficacious dose of a somatostatin mimetic for the conditions and diseases described herein and in general those benefitted by the direct nose-to-brain delivery of a somatostatin mimetic, is substantially lower than the dose required by a nasal route of administration that is not nose-to-brain administration or not formulated for nose-to-brain administration.

In some embodiments, the dose and dosing regimen described herein provides for an effective administration that is lower (e.g., lower total daily dose) than the intranasal dose and dosing regimen of the same somatostatin mimetic for treating other conditions and disease than those described herein, such as acromegaly and carcinoid syndrome. Such intranasal somatostatin mimetic pharmaceutical products have not been approved for treatments of these other diseases.

In some embodiments, the systemic exposure achieved by the intranasal administration of a somatostatin mimetic, and in particular the direct nose-to-brain route, as disclosed herein is lower than the systemic exposure achieved by another route of administration such as a parenteral (e.g., intravenous, subcutaneous, intradermal, intramuscular, intraperitoneal) or oral route. In some embodiments, the side effect profile and/or adverse events elicited by the intranasal administration of a somatostatin mimetic, and in particular the direct nose-to-brain route, as disclosed results in lower incidence and severity of side effects or adverse events than elicited by another route of administration such as a parenteral (e.g., intravenous, subcutaneous, intradermal, intramuscular, intraperitoneal) or oral route. In some embodiments, systemic absorption of the somatostatin mimetic is essentially avoided by the intranasal administration, and in particular the direct nose-to-brain route, as disclosed herein.

In some embodiments, the systemic exposure achieved by direct nose-to-brain administration of a somatostatin mimetic, as disclosed herein is lower than the systemic exposure achieved by the traditional intranasal route of administration.

In some embodiments, the dose of effective intranasal amount of somatostatin mimetic is equal to or less than about 300 mcg per day, equal to or less than about 250 mcg per day, equal to or less than about 200 mcg per day, equal to or less than about 150 mcg per day, equal to or less than about 100 mcg per day, equal to or less than about 90 mcg per day, equal to or less than about 80 mcg per day, equal to or less than about 70 mcg per day, equal to or less than about 60 mcg per day, equal to or less than about 50 mcg per day, equal to or less than about 40 mcg per day, equal to or less than about 30 mcg per day, equal to or less than about 20 mcg per day, equal to or less than about 10 mcg per day or equal to or less than about 5 mcg per day, equal to or less than about 1 mcg per day or greater than or equal to about 0.5 mcg per day. In some embodiments, the foregoing doses are administered once a day. In some embodiments, a foregoing dose is administered as a divided dose twice a day. In some embodiments, a foregoing dose is administered as a divided dose three times a day. In some embodiments, a foregoing dose is administered as a divided dose four times a day. In some embodiments, a foregoing dose is administered every other day or every third day. In some embodiments, a foregoing dose is administered once a week. For any of the aforementioned doses, less than may extend to the next lower disclosed dose, or to any of the lower disclosed doses described. By way of example, less than or equal to about 60 mcg per day also refers to about 5-60, about 10-60, about 20-60, about 30-60, about 40-60 or about 50-60 mcg per day.

In some embodiments, the serum area-under-the-curve after a single dose of an effective dosing regimen of a somatostatin mimetic administered by the intranasal route is lower than the serum area-under-the-curve after a single dose of an effective dosing regimen of the somatostatin mimetic administered by a route other than intranasal, such as but not limited to subcutaneous, intramuscular, oral and intravenous.

In some embodiments, the serum area-under-the-curve after a single dose and dosing regimen described herein provides for an effective administration that is lower than the serum area-under-the-curve from a subcutaneous dose and dosing regimen of the same somatostatin mimetic. In some embodiments, the serum area-under-the-curve from the dose and dosing regimen described herein provides for an effective administration that is lower than the serum area-under-the-curve from the intravenous dose and dosing regimen of the same somatostatin mimetic. In some embodiments, the serum area-under-the-curve from the dose and dosing regimen described herein provides for an effective administration that is lower than the serum area-under-the-curve from the intramuscular dose and dosing regimen of the same somatostatin mimetic. In some embodiments, the serum area-under-the-curve from the dose and dosing regimen described herein provides for an effective administration that is lower than the serum area-under-the-curve from the deep subcutaneous dose and dosing regimen of the same somatostatin mimetic. In some embodiments, the serum area-under-the-curve from the dose and dosing regimen described herein provides for an effective administration that is lower than the serum area-under-the-curve from the depot dose and dosing regimen of the same somatostatin mimetic. In some embodiments, the serum area-under-the-curve from the dose and dosing regimen described herein provides for an effective administration that is lower than the serum area-under-the-curve from the oral dose and dosing regimen of the same somatostatin mimetic. In any of the foregoing embodiments, the lower dose and dosing regimen is for treating the same condition or disease.

In some embodiments, the serum area-under-the-curve of the intranasal dose of the somatostatin mimetic is about equal to or less than about 50% of the serum area-under-the-curve of the somatostatin mimetic administered by a non-intranasal route. In some embodiments, the serum area-under-the-curve of the intranasal dose of the somatostatin mimetic is about equal to or less than about 45% of the serum area-under-the-curve of the somatostatin mimetic administered by a non-intranasal route. In some embodiments, the serum area-under-the-curve of the intranasal dose of the somatostatin mimetic is about equal to or less than about 40% of the serum area-under-the-curve of the somatostatin mimetic administered by a non-intranasal route. In some embodiments, the serum area-under-the-curve of the intranasal dose of the somatostatin mimetic is about equal to or less than about 35% of the serum area-under-the-curve of the somatostatin mimetic administered by a non-intranasal route. In some embodiments, the serum area-under-the-curve of the intranasal dose of the somatostatin mimetic is about equal to or less than about 30% of the serum area-under-the-curve of the somatostatin mimetic administered by a non-intranasal route. In some embodiments, the serum area-under-the-curve of the intranasal amount of the somatostatin mimetic is about equal to or less than about 25% of the serum area-under-the-curve of the somatostatin mimetic administered by a non-intranasal route. In some embodiments, the serum area-under-the-curve of the intranasal dose of the somatostatin mimetic is about equal to or less than about 20% of the serum area-under-the-curve of the somatostatin mimetic administered by a non-intranasal route. In some embodiments, the serum area-under-the-curve of the intranasal dose of the somatostatin mimetic is about equal to or less than about 15% of the serum area-under-the-curve of the somatostatin mimetic administered by a non-intranasal route. In some embodiments, the serum area-under-the-curve of the intranasal amount of the somatostatin mimetic is about equal to or less than about 10% of the serum area-under-the-curve of the somatostatin mimetic administered by a non-intranasal route. In some embodiments, the serum area-under-the-curve of the intranasal dose of the somatostatin mimetic is about equal to or less than about 5% of the serum area-under-the-curve of the somatostatin mimetic administered by a non-intranasal route. In some embodiments, the serum area-under-the-curve of the intranasal dose of the somatostatin mimetic is about equal to or less than about 4% of the serum area-under-the-curve of the somatostatin mimetic administered by a non-intranasal route. In some embodiments, the serum area-under-the-curve of the intranasal dose of the somatostatin mimetic is about equal to or less than about 3% of the serum area-under-the-curve of the somatostatin mimetic administered by a non-intranasal route. In some embodiments, the serum area-under-the-curve of the intranasal dose of the somatostatin mimetic is about equal to or less than about 2% of the serum area-under-the-curve of the somatostatin mimetic administered by a non-intranasal route. In some embodiments, the serum area-under-the-curve of the intranasal dose of the somatostatin mimetic is about equal to or less than about 1% of the serum area-under-the-curve of the somatostatin mimetic administered by a non-intranasal route. For any of the aforementioned percentages, less than may extend to the next lower disclosed percentage, or to any of the lower disclosed percentages described. By way of example, less than or equal to about 25% also refers to about 1-25%, about 2-25%, about 3-25%, about 4-25%, about 5-25%, about 10-25%, about 15-25%, or about 20-25%.

In some embodiments, the serum area-under-the-curve from a single intranasal dose of a somatostatin mimetic is equal to or less than about 900 mcg L−1 min. In some embodiments, the serum area-under-the-curve from a single intranasal dose of a somatostatin mimetic is equal to or less than about 800 mcg L−1 min. In some embodiments, the serum area-under-the-curve from a single intranasal dose of a somatostatin mimetic is equal to or less than about 700 mcg L−1 min. In some embodiments, the serum area-under-the-curve from a single intranasal dose of a somatostatin mimetic is equal to or less than about 600 mcg L−1 min. In some embodiments, the serum area-under-the-curve from a single intranasal dose of a somatostatin mimetic is equal to or less than about 500 mcg L−1 min. In some embodiments, the serum area-under-the-curve from a single intranasal dose of a somatostatin mimetic is equal to or less than about 400 mcg L−1 min. In some embodiments, the serum area-under-the-curve from a single intranasal dose of a somatostatin mimetic is equal to or less than about 300 mcg L−1 min. In some embodiments, the serum area-under-the-curve from a single intranasal dose of a somatostatin mimetic is equal to or less than about 200 mcg L−1 min. In some embodiments, the serum area-under-the-curve from a single intranasal dose of a somatostatin mimetic is equal to or less than about 100 mcg L−1 min. In some embodiments, the serum area-under-the-curve from a single intranasal dose of a somatostatin mimetic is equal to or less than about 90 mcg L−1 min. In some embodiments, the serum area-under-the-curve from a single intranasal dose of a somatostatin mimetic is equal to or less than about 80 mcg L−1 min. In some embodiments, the serum area-under-the-curve from a single intranasal dose of a somatostatin mimetic is equal to or less than about 70 mcg L−1 min. In some embodiments, the serum area-under-the-curve from a single intranasal dose of a somatostatin mimetic is equal to or less than about 60 mcg L−1 min. In some embodiments, the serum area-under-the-curve from a single intranasal dose of a somatostatin mimetic is equal to or less than about 50 mcg L−1 min. In some embodiments, the serum area-under-the-curve from a single intranasal dose of a somatostatin mimetic is equal to or less than about 40 mcg L−1 min. In some embodiments, the serum area-under-the-curve from a single intranasal dose of a somatostatin mimetic is equal to or less than about 30 mcg L−1 min. In some embodiments, the serum area-under-the-curve from a single intranasal dose of a somatostatin mimetic is equal to or less than about 20 mcg L−1 min. In some embodiments, the serum area-under-the-curve from a single intranasal dose of a somatostatin mimetic is equal to or less than about 10 mcg L−1 min. In some embodiments, the serum area-under-the-curve from a single intranasal dose of a somatostatin mimetic is equal to or less than about 5 mcg L−1 min. For any of the aforementioned AUCs, less than may extend to the next lower disclosed AUC, or to any of the lower disclosed AUCs described. By way of example, less than or equal to about 30 mcg L−1 min also refers to about 5-30, about 10-30 or about 20-30 mcg L−1 min.

In some embodiments, the serum area-under-the-curve after a single dose of an effective dosing regimen of a somatostatin mimetic administered by the intranasal route is lower than the serum area-under-the-curve after a single dose of an effective dosing regimen of the somatostatin mimetic administered for another indication by the intranasal route. Non-limiting examples of other indications include acromegaly and carcinoid syndrome.

In some embodiments, the serum area-under-the-curve of the intranasal amount of the somatostatin mimetic is about equal to or less than about 50% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 45% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 40% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 35% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 30% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 25% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 20% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 15% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 10% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 5% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 4% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 3% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 2% of the serum area-under-the-curve of the somatostatin mimetic administered by the intranasal route for an indication other than IIH or cluster headache. In some embodiments, the serum area-under-the-curve of the intranasal amount of the somatostatin mimetic is about equal to or less than about 1% of the serum area-under-the-curve of the somatostatin mimetic administered by the intranasal route for an indication other than IIH or cluster headache. For any of the aforementioned percentages, less than may extend to the next lower disclosed percentage, or to any of the lower disclosed percentages described. By way of example, less than or equal to about 25% also refers to about 1-25%, about 2-25%, about 3-25%, about 4-25%, about 5-25%, about 10-25%, about 15-25%, or about 20-25%.

Thus, a pharmaceutical composition in unit dose form may comprise about equal to or less than about 300 mcg per unit dose, equal to or less than about 250 mcg per unit dose, equal to or less than about 200 mcg per unit dose, equal to or less than about 150 mcg per unit dose, equal to or less than about 100 mcg per unit dose, equal to or less than about 90 mcg per unit dose, equal to or less than about 80 mcg per unit dose, equal to or less than about 70 mcg per unit dose, equal to or less than about 60 mcg per unit dose, equal to or less than about 50 mcg per unit dose, equal to or less than about 40 mcg per unit dose, equal to or less than about 30 mcg per unit dose, equal to or less than about 20 mcg per unit dose, equal to or less than about 10 mcg per unit dose or equal to or less than about 5 mcg per unit dose, and greater than about 1 mcg per unit dose. For any of the aforementioned unit doses, less than may extend to the next lower disclosed unit dose, or to any of the lower disclosed unit doses described. By way of example, less than or equal to about 80 mcg per unit dose also refers to about 1-80, 5-80, about 10-80, about 20-80, about 30-80, about 40-80 or about 50-80 mcg per unit dose.

### Carbonic Anhydrase Inhibitors

Carbonic anhydrase inhibitors are a class of pharmaceutical agents that suppress the activity of carbonic anhydrase. They are approved for indications including glaucoma and use as diuretics. Off-label uses include antiepileptics, in the management of mountain sickness, gastric and duodenal ulcers, idiopathic intracranial hypertension, neurological disorders, and osteoporosis.

Non-limiting examples of carbonic anhydrase inhibitors include topiramate (TOPAMAX), acetazolamide (acetazolamide sodium) (DIAMOX), methazolamide (NEPTAZANE), zonisamide (ZONEGRAN), sulthiame (OSPOLOT) and bichlorphenamide (diclofenamide; KEVEYIS). Carbonic anhydrase inhibitors includes the aforementioned generic names, proprietary names (whether active or discontinued), synonyms thereof, and are intended to be exemplary and non-limiting.

### Topiramate

Topiramate (2,3:4,5-bis-O-(1-methylethylidene)-beta-D-fructopyranos e sulfamate; TOPAMAX) is an antiepileptic agent (or AED; anti-epileptic drug) indicated for (approved indications): monotherapy epilepsy: initial monotherapy in patients ≥2 years of age with partial onset or primary generalized tonic-clonic seizures. It is also indicated for adjunctive therapy epilepsy: adjunctive therapy for adults and pediatric patients (2 to 16 years of age) with partial onset seizures or primary generalized tonic-clonic seizures, and in patients >2 years of age with seizures associated with Lennox-Gastaut syndrome (LGS). It is also indicated for migraine: treatment for adults for prophylaxis of migraine headache.

The recommended dosage for epilepsy monotherapy in adults is 400 mg/day in two divided doses, increased weekly by increments of 50 mg for the first 4 weeks then 100 mg for weeks 5 to 6. For epilepsy adjunctive therapy in adults with partial onset seizures of Lennox-Gastaut syndrome (LGS), 200-400 mg/day in two divided doses. The dosage should be increased weekly to an effective dose by increments of 25 to 50 mg. For epilepsy adjunctive therapy in adults with primary generalized tonic-clonic seizures, 400 mg/day in two divided doses. The dosage should be increased weekly to an effective dose by increments of 25 to 50 mg. For migraine, 100 mg/day in two divided doses. The dosage should be increased weekly by increments of 25 mg. Dose and titration should be guided by clinical outcome.

Taken orally, topiramate is available as tablets of 15 mg, 25 mg, 50 mg, 100 mg or 200 mg, and as Sprinkle Capsules at 15 mg and 25 mg. Extended release capsules are available at 25 mg, 50 mg, 100 mg, 150 mg, 200 mg.

The product label lists the following warnings and precautions: Acute myopia and secondary angle closure glaucoma: Untreated elevated intraocular pressure can lead to permanent visual loss. The primary treatment to reverse symptoms is discontinuation of TOPAMAX® as rapidly as possible; Oligohidrosis and hyperthermia: Monitor decreased sweating and increased body temperature, especially in pediatric patients; Metabolic acidosis: Baseline and periodic measurement of serum bicarbonate is recommended. Consider dose reduction or discontinuation of TOPAMAX® if clinically appropriate; Suicidal behavior and ideation: Antiepileptic drugs increase the risk of suicidal behavior or ideation; Cognitive/neuropsychiatric: TOPAMAX® may cause cognitive dysfunction. Patients should use caution when operating machinery including automobiles. Depression and mood problems may occur in epilepsy and migraine populations; Fetal Toxicity: TOPAMAX® use during pregnancy can cause cleft lip and/or palate; Withdrawal of AEDs: Withdrawal of TOPAMAX® should be done gradually; Hyperammonemia and encephalopathy associated with or without concomitant valproic acid use: Patients with inborn errors of metabolism or reduced mitochondrial activity may have an increased risk of hyperammonemia. Measure ammonia if encephalopathic symptoms occur; Kidney stones: Use with other carbonic anhydrase inhibitors, other drugs causing metabolic acidosis, or in patients on a ketogenic diet should be avoided; Hypothermia has been reported with and without hyperammonemia during topiramate treatment with concomitant valproic acid use. The foregoing are referred to herein as adverse effect or side effects.

The product label provides the following adverse reactions. The most common (>5% more frequent than placebo or low-dose TOPAMAX® in monotherapy) adverse reactions in controlled, epilepsy clinical trials were paresthesia, anorexia, weight decrease, fatigue, dizziness, somnolence, nervousness, psychomotor slowing, difficulty with memory, difficulty with concentration/attention, cognitive problems, confusion, mood problems, fever, infection, and flushing. The most common (>5% more frequent than placebo) adverse reactions in controlled, migraine clinical trials were paresthesia and taste perversion.

Because of side effects, patients taking topiramate should avoid activities requiring mental alertness and coordination until drug effects are realized. Topiramate may impair heat regulation, especially in children. Caution should be used with activities leading to an increased core temperature, such as strenuous exercise, exposure to extreme heat, or dehydration. Topiramate may cause visual field defects, and decrease effectiveness of estrogen-containing oral contraceptives. Taking topiramate in the first trimester of pregnancy may increase risk of cleft lip/cleft palate in infant. Studies of topiramate in IIH reported side effects including distal paresthesias, difficulties in concentrating and prominent weight loss.

Thus, in various embodiments herein, the dose of topiramate for treatment of IIH or cluster headache in combination with a somatostatin mimetic, includes unit doses of 200, 100, 50, 25, 20, 15, 10 or 5 mg.

In some embodiments, the dosing regimen of topiramate is about 200 mg twice a day. In some embodiments, the dosing regimen of topiramate is about 100 mg twice a day. In some embodiments, the dosing regimen of topiramate is about 50 mg twice a day. In some embodiments, the dosing regimen of topiramate is about 25 mg twice a day. In some embodiments, the dosing regimen of topiramate is about 20 mg twice a day. In some embodiments, the dosing regimen of topiramate is about 15 mg twice a day. In some embodiments, the dosing regimen of topiramate is about 10 mg twice a day. In some embodiments, the dosing regimen of topiramate is about 5 mg twice a day.

In some embodiments, the dosing regimen of topiramate is about 200 mg once a day. In some embodiments, the dosing regimen of topiramate is about 100 mg once a day. In some embodiments, the dosing regimen of topiramate is about 50 mg once a day. In some embodiments, the dosing regimen of topiramate is about 25 mg once a day. In some embodiments, the dosing regimen of topiramate is about 20 mg once a day. In some embodiments, the dosing regimen of topiramate is about 15 mg once a day. In some embodiments, the dosing regimen of topiramate is about 10 mg once a day. In some embodiments, the dosing regimen of topiramate is about 5 mg once a day.

### Acetazolamide

Acetazolamide (N-(5-sulfamoyl-1,3,4-thiadiazol-2-yl) acetamide) (acetazolamide sodium), sold under the trade name DIAMOX among others, is a medication that is effective in (approved indications): the control of fluid secretion (e.g., some types of glaucoma), in the treatment of certain convulsive disorders (e.g., epilepsy) and in the promotion of diuresis in instances of abnormal fluid retention (e.g., cardiac edema). It is indicated for (approved indications): adjunctive treatment of: chronic simple (open-angle) glaucoma, secondary glaucoma, and preoperatively in acute angle-closure glaucoma where delay of surgery is desired in order to lower intraocular pressure. DIAMOX is also indicated for (approved indications): the prevention or amelioration of symptoms associated with acute mountain sickness despite gradual ascent.

DIAMOX SEQUELS (and generic versions) (acetazolamide extended-release [ER] capsules) are available as 500 mg capsules. Acetazolamide (acetazolamide sodium) (DIAMOX) oral tablets are available as 125 mg and 500 mg tablets. Acetazolamide for intravenous injection is available at 500 mg per vial for reconstitution with at least 5 mL Sterile Water for Injection.

The recommended dosage is 1 capsule (500 mg) two times a day. For the aforementioned indications, usually 1 capsule is administered in the morning and 1 capsule in the evening. It may be necessary to adjust the dose, but it has usually been found that dosage in excess of 2 capsules (1 g) does not produce an increased effect. The dosage should be adjusted with careful individual attention both to symptomatology and intraocular tension. In all cases, continuous supervision by a physician is advisable. In those unusual instances where adequate control is not obtained by the twice-a-day administration of DIAMOX SEQUELS, the desired control may be established by means of DIAMOX (tablets or parenteral). Use tablets or parenteral in accordance with the more frequent dosage schedules recommended for these dosage forms, such as 250 mg every four hours, or an initial dose of 500 mg followed by 250 mg or 125 mg every four hours, depending on the case in question.

For acute altitude sickness, the recommended dosage is 500 mg to 1000 mg daily, in divided doses using tablets or extended-release capsules as appropriate. In circumstances of rapid ascent, such as in rescue or military operations, the higher dose level of 1000 mg is recommended. It is preferable to initiate dosing 24 to 48 hours before ascent and to continue for 48 hours while at high altitude, or longer as necessary to control symptoms.

The acetazolamide label lists the following warnings: Fatalities have occurred, although rarely, due to severe reactions to sulfonamides including Stevens-Johnson syndrome, toxic epidermal necrolysis, fulminant hepatic necrosis, anaphylaxis, agranulocytosis, aplastic anemia, and other blood dyscrasias. Sensitizations may recur when a sulfonamide is readministered irrespective of the route of administration. If signs of hypersensitivity or other serious reactions occur, discontinue use of this drug. Caution is advised for patients receiving concomitant high-dose aspirin and DIAMOX, as anorexia, tachypnea, lethargy, metabolic acidosis, coma, and death have been reported. Information for Patients includes: Adverse reactions common to all sulfonamide derivatives may occur: anaphylaxis, fever, rash (including erythema multiforme, Steven-Johnson syndrome, toxic epidermal necrolysis), crystalluria, renal calculus, bone marrow depression, thrombocytopenic purpura, hemolytic anemia, leukopenia, pancytopenia, and agranulocytosis. In patients with pulmonary obstruction or emphysema where alveolar ventilation may be impaired, DIAMOX which may precipitate or aggravate acidosis should be used with caution. Gradual ascent is desirable to try to avoid acute mountain sickness. If rapid ascent is undertaken and DIAMOX is used, it should be noted that such use does not obviate the need for prompt descent if severe forms of high altitude sickness occur, i.e., high altitude pulmonary edema (HAPE) or high altitude cerebral edema. Caution is advised for patients receiving concomitant high-dose aspirin and DIAMOX, as anorexia, tachypnea, lethargy, metabolic acidosis, coma, and death have been reported (see WARNINGS). Both increases and decreases in blood glucose have been described in patients treated with acetazolamide. This should be taken into consideration in patients with impaired glucose tolerance or diabetes mellitus. Acetazolamide treatment may cause electrolyte imbalances, including hyponatremia and hypokalemia, as well as metabolic acidosis. Therefore, periodic monitoring of serum electrolytes is recommended. Particular caution is recommended in patients with conditions that are associated with, or predispose a patient to, electrolyte and acid/base imbalances, such as patients with impaired renal function (including elderly patients), patients with diabetes mellitus, and patients with impaired alveolar ventilation. Some adverse reactions to acetazolamide, such as drowsiness, fatigue, and myopia, may impair the ability to drive and operate machinery.

Common side effects of acetazolamide include numbness, ringing in the ears, loss of appetite, vomiting, and sleepiness. It is not recommended in those with significant kidney problems, liver problems, or who are allergic to sulfonamides. Studies on acetazolamide in IIH reported side effects including fatigue and tingling of the hands and feet.

Thus, in various embodiments herein, the dose of acetazolamide for treatment of IIH or cluster headache in combination with a somatostatin mimetic, includes unit doses of 500, 250, 125, 100, 50, 25, 20, 15, 10 or 5 mg.

In some embodiments, the dosing regimen of acetazolamide is about 500 mg twice a day. In some embodiments, the dosing regimen of acetazolamide is about 125 mg twice a day. In some embodiments, the dosing regimen of acetazolamide is about 100 mg twice a day. In some embodiments, the dosing regimen of acetazolamide is about 50 mg twice a day. In some embodiments, the dosing regimen of acetazolamide is about 25 mg twice a day. In some embodiments, the dosing regimen of acetazolamide is about 20 mg twice a day. In some embodiments, the dosing regimen of acetazolamide is about 15 mg twice a day. In some embodiments, the dosing regimen of acetazolamide is about 10 mg twice a day. In some embodiments, the dosing regimen of acetazolamide is about 5 mg twice a day.

In some embodiments, the dosing regimen of acetazolamide is about 500 mg once a day. In some embodiments, the dosing regimen of acetazolamide is about 125 mg once a day. In some embodiments, the dosing regimen of acetazolamide is about 100 mg once a day. In some embodiments, the dosing regimen of acetazolamide is about 50 mg once a day. In some embodiments, the dosing regimen of acetazolamide is about 25 mg once a day. In some embodiments, the dosing regimen of acetazolamide is about 20 mg once a day. In some embodiments, the dosing regimen of acetazolamide is about 15 mg once a day. In some embodiments, the dosing regimen of acetazolamide is about 10 mg once a day. In some embodiments, the dosing regimen of acetazolamide is about 5 mg once a day.

### Methazolamide

Methazolamide (NEPTAZANE and generic) is a carbonic anhydrase inhibitor indicated for (approved indications): the treatment of chronic open-angle or secondary glaucoma; short-term therapy of acute angle-closure glaucoma prior to surgery. It is available in 25 and 50 mg oral dosage forms. The recommended dose regimen is 50 to 100 mg 2-3 times a day.

Adverse reactions include, by system: Central nervous system: Confusion, drowsiness, fatigue, flaccid paralysis, malaise, paresthesia, seizure; Dermatologic: Erythema multiforme, skin photosensitivity, skin rash, Stevens-Johnson syndrome, toxic epidermal necrolysis, urticaria; Endocrine & metabolic: Electrolyte disturbance, glycosuria, metabolic acidosis; Gastrointestinal: Decreased appetite, diarrhea, dysgeusia, melena, nausea, vomiting; Genitourinary: Crystalluria, hematuria; Hematologic & oncologic: Agranulocytosis, aplastic anemia, bone marrow depression, hemolytic anemia, immune thrombocytopenia, leukopenia, pancytopenia; Hepatic: Fulminant hepatic necrosis, hepatic insufficiency; Hypersensitivity: Anaphylaxis, hypersensitivity reaction; Ophthalmic: Myopia; Otic: Auditory disturbance, tinnitus; and Renal: Nephrolithiasis, polyuria.

Warnings and precautions include: CNS effects: May impair mental alertness and/or physical coordination; Electrolyte disturbance: Initially, potassium excretion may be increased; periodically monitor serum electrolytes and signs of hypokalemia in at risk patients; Sulfonamide (“sulfa”) allergy. Disease-related concerns include: Diabetes: Use with caution in patients with prediabetes or diabetes mellitus; may see a change in glucose control; Hepatic impairment: Use with caution in patients with hepatic impairment; may precipitate hepatic encephalopathy. Use is contraindicated in patients with marked liver impairment or cirrhosis; Respiratory disease: Use with caution in patients with respiratory disease such as emphysema or pulmonary obstruction; may precipitate or aggravate respiratory acidosis.

Common side effects include: nausea, vomiting, diarrhea, change in taste, lack of appetite, fatigue and loss of strength and energy.

Thus, in various embodiments herein, the dose of methazolamide for treatment of IIH or cluster headache in combination with a somatostatin mimetic, includes unit doses of 50, 25, 20, 15, 10 or 5 mg.

In some embodiments, the dosing regimen of methazolamide is about 50 mg three times a day. In some embodiments, the dosing regimen of methazolamide is about 25 mg three times a day. In some embodiments, the dosing regimen of methazolamide is about 20 mg three times a day. In some embodiments, the dosing regimen of methazolamide is about 15 mg three times a day. In some embodiments, the dosing regimen of methazolamide is about 10 mg three times a day. In some embodiments, the dosing regimen of methazolamide is about 5 mg three times a day.

In some embodiments, the dosing regimen of methazolamide is about 50 mg twice a day. In some embodiments, the dosing regimen of methazolamide is about 25 mg twice a day. In some embodiments, the dosing regimen of methazolamide is about 20 mg twice a day. In some embodiments, the dosing regimen of methazolamide is about 15 mg twice a day. In some embodiments, the dosing regimen of methazolamide is about 10 mg twice a day. In some embodiments, the dosing regimen of methazolamide is about 5 mg twice a day.

In some embodiments, the dosing regimen of methazolamide is about 50 mg once a day. In some embodiments, the dosing regimen of methazolamide is about 25 mg once a day. In some embodiments, the dosing regimen of methazolamide is about 20 mg once a day. In some embodiments, the dosing regimen of methazolamide is about 15 mg once a day. In some embodiments, the dosing regimen of methazolamide is about 10 mg once a day. In some embodiments, the dosing regimen of methazolamide is about 5 mg once a day.

### Zonisamide

Zonisamide (ZONEGRAN) is an antiseizure drug indicated as (approved indications): adjunctive therapy in the treatment of partial seizures in adults with epilepsy. It is supplied for oral administration as capsules containing 25 mg, 50 mg or 100 mg zonisamide.

The most commonly observed adverse events associated with the use of ZONEGRAN in controlled clinical trials that were not seen at an equivalent frequency among placebo-treated patients were somnolence, anorexia, dizziness, headache, nausea, and agitation/irritability.

Thus, in various embodiments herein, the dose of zonisamide for treatment of IIH or cluster headache in combination with a somatostatin mimetic, includes unit doses of 100, 50, 25, 20, 15, 10 or 5 mg.

In some embodiments, the dosing regimen of zonisamide is about 100 mg twice a day. In some embodiments, the dosing regimen of zonisamide is about 50 mg twice a day. In some embodiments, the dosing regimen of zonisamide is about 25 mg twice a day. In some embodiments, the dosing regimen of zonisamide is about 20 mg twice a day. In some embodiments, the dosing regimen of zonisamide is about 15 mg twice a day. In some embodiments, the dosing regimen of zonisamide is about 10 mg twice a day. In some embodiments, the dosing regimen of zonisamide is about 5 mg twice a day.

In some embodiments, the dosing regimen of zonisamide is about 100 mg once a day. In some embodiments, the dosing regimen of zonisamide is about 50 mg once a day. In some embodiments, the dosing regimen of zonisamide is about 25 mg once a day. In some embodiments, the dosing regimen of zonisamide is about 20 mg once a day. In some embodiments, the dosing regimen of zonisamide is about 15 mg once a day. In some embodiments, the dosing regimen of zonisamide is about 10 mg once a day. In some embodiments, the dosing regimen of zonisamide is about 5 mg once a day.

### Sulthiame

Sulthiame or sultiame (p-(tetrahydro-2H-1,2-thiazin-2-yl)benzenesulfonamide, S,S-dioxide; OSPOLOT) is indicated for (approved indications): anticonvulsant for behavioral being tested for the treatment of epileptic seizures of focal origin with or without secondary generalization, especially benign partial epilepsies in childhood, such as rolandic epilepsy, pseudo-Lennox syndrome, and bioelectric status epilepticus in non-REM sleep (ESES), Landau-Kleffner syndrome. Film coated tablets for oral administration contain 200 mg of sulthiame (the tables are scored for making 100 mg doses). The maintenance dose is about 5 to 10 mg/kg body weight per day (i.e., 350-700 mg/day in a 70 kg individual).

Side effects of sulthiame include gastric complaints can occur in about 10% of patients. The following side effects occasionally occur dose-dependent: paresthesias in the extremities and in the face, tachypnea, hyperpnea, dyspnea, dizziness, headache, stenocardia, tachycardia, double vision, singultus, weight loss or lack of appetite. In rare cases, hallucinations, anxiety, myasthenic phenomena, lack of drive, joint pain, a grand mal state or increased seizure activity can be triggered. In individual cases, it has been suspected that sulthiame may be associated with the induction of acute renal failure, Stevens-Johnson syndrome, Lyell's syndrome, or polyneuritis. In one case, the administration of OSPOLOT led to progressive weakness of the limbs, hypersalivation, slurred speech, increasing drowsiness up to coma. The symptoms abated within hours of OSPOLOT being discontinued.

Thus, in various embodiments herein, the dose of sulthiame for treatment of IIH or cluster headache in combination with a somatostatin mimetic, includes unit doses of 200, 100, 50, 25 or 10 mg.

In some embodiments, the dosing regimen of sulthiame is about 200 mg three times a day. In some embodiments, the dosing regimen of sulthiame is about 100 mg three times a day. In some embodiments, the dosing regimen of sulthiame is about 50 mg three times a day. In some embodiments, the dosing regimen of sulthiame is about 25 mg three times a day. In some embodiments, the dosing regimen of sulthiame is about 20 mg three times a day. In some embodiments, the dosing regimen of sulthiame is about 15 mg three times a day. In some embodiments, the dosing regimen of sulthiame is about 10 mg three times a day. In some embodiments, the dosing regimen of sulthiame is about 5 mg three times a day.

In some embodiments, the dosing regimen of sulthiame is about 200 mg twice a day. In some embodiments, the dosing regimen of sulthiame is about 100 mg twice a day. In some embodiments, the dosing regimen of sulthiame is about 50 mg twice a day. In some embodiments, the dosing regimen of sulthiame is about 25 mg twice a day. In some embodiments, the dosing regimen of sulthiame is about 20 mg twice a day. In some embodiments, the dosing regimen of sulthiame is about 15 mg twice a day. In some embodiments, the dosing regimen of sulthiame is about 10 mg twice a day. In some embodiments, the dosing regimen of sulthiame is about 5 mg twice a day.

In some embodiments, the dosing regimen of sulthiame is about 200 mg once a day. In some embodiments, the dosing regimen of sulthiame is about 100 mg once a day. In some embodiments, the dosing regimen of sulthiame is about 50 mg once a day. In some embodiments, the dosing regimen of sulthiame is about 25 mg once a day. In some embodiments, the dosing regimen of sulthiame is about 20 mg once a day. In some embodiments, the dosing regimen of sulthiame is about 15 mg once a day. In some embodiments, the dosing regimen of sulthiame is about 10 mg once a day. In some embodiments, the dosing regimen of sulthiame is about 5 mg once a day.

### Bichlorphenamide

Bichlorphenamide (KEVEYIS) is a carbonic anhydrase inhibitor indicated for (approved indications): treatment of primary hyperkalemic periodic paralysis, primary hypokalemic periodic paralysis, and related variants. Tablets of 50 mg are available. The recommended dosage is an initial dose: 50 mg twice daily, then titrate the dose based on individual response. The maximum recommended dose is 200 mg daily.

In a 9-week randomized controlled trial in adults with hyperkalemic or hypokalemic periodic paralysis, the most common adverse reactions in patients treated with KEVEYIS with rates greater than placebo, were paresthesia, cognitive disorder, dysgeusia, and confusional state.

Thus, in various embodiments herein, the dose of bichlorphenamide for treatment of IIH or cluster headache in combination with a somatostatin mimetic, includes unit doses of 50, 25, 20, 15, 10 or 5 mg.

In some embodiments, the dosing regimen of bichlorphenamide is about 50 mg twice a day. In some embodiments, the dosing regimen of bichlorphenamide is about 25 mg twice a day. In some embodiments, the dosing regimen of bichlorphenamide is about 20 mg twice a day. In some embodiments, the dosing regimen of bichlorphenamide is about 15 mg twice a day. In some embodiments, the dosing regimen of bichlorphenamide is about 10 mg twice a day. In some embodiments, the dosing regimen of bichlorphenamide is about 5 mg twice a day.

In some embodiments, the dosing regimen of bichlorphenamide is about 50 mg once a day. In some embodiments, the dosing regimen of bichlorphenamide is about 25 mg once a day. In some embodiments, the dosing regimen of bichlorphenamide is about 20 mg once a day. In some embodiments, the dosing regimen of bichlorphenamide is about 15 mg once a day. In some embodiments, the dosing regimen of bichlorphenamide is about 10 mg once a day. In some embodiments, the dosing regimen of bichlorphenamide is about 5 mg once a day.

Unit Dose and Administration of Carbonic Anhydrase inhibitors

In some embodiments, a unit dose of a carbonic anhydrase inhibitor for use in the methods and therapeutic combinations described herein may be a single dose of compound at the dosage level that is administered as prescribed for approved indications, but administered at a lower frequency than prescribed for approved indications. In some embodiments, a unit dose of a carbonic anhydrase inhibitor for use in the methods and therapeutic combinations described herein may be a single dose of compound at a lower dose level than prescribed for approved indications, administered at a frequency prescribed for approved indications. In some embodiment, a unit dose of a carbonic anhydrase inhibitor for use in the methods and therapeutic combinations described herein may be both a lower single dose level of compound that is administered as prescribed for approved indications, and is administered at a lower frequency than prescribed for approved indications. In certain embodiments, the lower dose levels and/or lower dosing frequency elicits fewer if any side effects known or associated with the compound, while providing efficacy in IIH or cluster headaches when administered with an intranasal formulation of somatostatin mimetic.

As noted above, the therapeutic combination and methods disclosed herein contemplates each of the components of the combination being administered by a different route of administration. The somatostatin mimetic is administered intranasally. The carbonic anhydrase inhibitor is typically administered orally, though dose forms for other routes of administration are available (e.g., intramuscular), and dose forms for other routes such as but not limited to intranasal (e.g., traditional or nose-to-brain) are embraced herein. In some embodiments, the somatostatin mimetic and carbonic anhydrase inhibitor are formulated together and administered in the same formulation.

In some embodiments, the therapeutic combination is provided to effectively treat IIH or cluster headache in patients in which intranasal administration of a somatostatin mimetic alone is insufficiently effective. In some embodiments, the therapeutic combination is provided to effectively treat IIH or cluster headache in patients in which administration of a carbonic anhydrase inhibitor has an unacceptable side effect profile and may limit patient compliance. In some embodiments, the therapeutic combination is provided to effectively treat IIH or cluster headache in patients in which have not found benefit by any prior therapeutic regimen. In some embodiments, the therapeutic combination is provided to effectively treat IIH or cluster headache in patients who have failed treatment by another therapeutic regimen. In some embodiments, the therapeutic combination is provided to effectively treat IIH or cluster headache in patients in whom another therapeutic regimen has become less effective or ineffective.

In some embodiments, the therapeutic combination is provided to effectively treat IIH or cluster headache in a greater number or proportion of patients than the number or proportion of IIH or cluster headache patients in whom intranasal administration of a somatostatin mimetic alone is effective. In some embodiments, the therapeutic combination is provided to effectively treat IIH or cluster headache in patients and reduce the number or proportion of patients in which administration of a carbonic anhydrase inhibitor alone has side effect profile that may limit patient compliance.

Each component of the therapeutic combination is administered at a dose level and/or dosing frequency (referred to herein as dosing regimen) that is lower than is therapeutically effective, in the case of the somatostatin mimetic, when administered alone by a non-nasal route.

In the case of the carbonic anhydrase inhibitor, the dosing regimen is lower than is therapeutically effective when administered alone, or lower than the prescribed dosage. In one embodiment, the therapeutically effective dose is the dose used to treat IIH. In one embodiment, the therapeutically effective dose is the dose used to treat any approved indication, such as but not limited to migraine, epilepsy monotherapy, epilepsy adjunctive therapy, glaucoma or mountain (altitude) sickness.

As will also be described in further detail below, each component is administered at a dosing regimen that provides a lower systemic exposure than is therapeutically effective, in the case of the somatostatin mimetic, when administered alone by a non-nasal route, and in the case of a carbonic anhydrase inhibitor, a lower systemic exposure than therapeutically effective when administered alone. In some embodiments therapeutically effective means effective for treating IIH or cluster headache. In some embodiments therapeutically effective means effective for treatment of an approved indication.

In some embodiments, the dosing regimen of the carbonic anhydrase inhibitor that provides a dose level or exposure that is less than the therapeutically effective dose when administered alone, results in an improved side effect profile and/or reduces any one or more of the side effects that are reported on its product label.

Thus, in various embodiments, a unit dose of acetazolamide is 125 mg. In one embodiment, a unit dose of acetazolamide is 250 mg. In one embodiment the unit dose of acetazolamide is 500 mg. In some embodiments, the dosing regimen of the unit dose is once per day. In some embodiments, the dosing regimen of the unit dose is twice per day.

In various embodiments, a unit dose of topiramate is 15 mg, 25 mg, 50 mg, 100 mg or 200 mg. In some embodiments, the dosing regimen of the unit dose is once per day. In some embodiments, the dosing regimen of the unit dose is twice per day.

In various embodiments, a unit dose of methazolamide is 25 mg, 50 mg or 100 mg. In some embodiments, the dosing regimen of the unit dose is once per day. In some embodiments, the dosing regimen of the unit dose is twice per day.

In various embodiments, a unit dose of zonisamide is 25 mg, 50 mg or 100 mg. In some embodiments, the dosing regimen of the unit dose is once per day. In some embodiments, the dosing regimen of the unit dose is twice per day.

In various embodiments, a unit dose of sulthiame is 100 mg or 200 mg. In some embodiments, the dosing regimen of the unit dose is once per day. In some embodiments, the dosing regimen of the unit dose is twice per day.

In various embodiments, a unit dose of bichlorfenamide is 50 mg or 100 mg. In some embodiments, the dosing regimen of the unit dose is once per day. In some embodiments, the dosing regimen of the unit dose is twice per day.

As noted elsewhere herein, the unit dose level and/or the frequency of the unit dose administration of a carbonic anhydrase inhibitor is lower than that recommended for treatment of an indicated condition or disease. In some embodiments, the unit dose level of administration of a carbonic anhydrase inhibitor is lower than that recommended for treatment of an indicated condition or disease. In some embodiments, frequency of administration of a unit dose of a carbonic anhydrase inhibitor is lower than that recommended for treatment of an indicated condition or disease. In some embodiments, the unit dose level and the frequency of the unit dose administration of a carbonic anhydrase inhibitor are lower than that recommended for treatment of an indicated condition or disease.

In some embodiments, a lower dose level and/or dosing frequency elicits fewer side effects. In some embodiments, a lower dose level and/or dosing frequency elicits fewer side effects without reducing efficacy. In some embodiments, the dosing frequency is half as often as recommended for an approved indication. (e.g., once per day instead of twice per day; once every other day instead of once daily). Where a lower dosage form is available, a dose level of half or less of the indicated dose is administered at the recommended dosing frequency for an approved indication. In some embodiments, both a lower dose and a lower frequent dose frequency of a carbonic anhydrase inhibitor are provided.

In some embodiment, a unit dose of topiramate for use in the methods and therapeutic combinations described herein may be both a lower single dose of compound that is administered as prescribed for approved indications, and is administered at a lower frequency than prescribed for approved indications. In certain embodiments, the lower dose levels and/or lower dosing frequency elicits fewer if any side effects known or associated with the compound, while providing efficacy in IIH or cluster headaches when administered with an intranasal formulation of somatostatin mimetic.

In one embodiment, 200 mg topiramate is administered once a day. In one embodiment, 100 mg topiramate is administered twice a day. In one embodiment, 100 mg topiramate is administered once a day. In one embodiment, 100 mg topiramate is administered every other day. In one embodiment, 50 mg topiramate is administered twice a day. In one embodiment, 50 mg topiramate is administered once a day. In one embodiment, 25 mg topiramate is administered twice a day. In one embodiment, 25 mg topiramate is administered once a day. In one embodiment, 15 mg topiramate is administered twice a day. In one embodiment, 15 mg topiramate is administered once a day

In some embodiments, a unit dose of acetazolamide for use in the methods and therapeutic combinations described herein may be a single dose of compound at the dosage level that is administered as prescribed for approved indications, but administered at a lower frequency than prescribed for approved indications. In some embodiments, a unit dose of acetazolamide for use in the methods and therapeutic combinations described herein may be a single dose of compound at a lower dose than prescribed for approved indications, administered at a frequency prescribed for approved indications. In some embodiment, a unit dose of acetazolamide for use in the methods and therapeutic combinations described herein may be both a lower single dose of compound that is administered as prescribed for approved indications, and is administered at a lower frequency than prescribed for approved indications. In certain embodiments, the lower dose levels and/or lower dosing frequency elicits fewer if any side effects known or associated with the compound, while providing efficacy in IIH or cluster headaches when administered with an intranasal formulation of somatostatin mimetic,

In one embodiment, 500 mg acetazolamide is administered twice a day. In one embodiment, 500 mg acetazolamide is administered once a day. In one embodiment, 500 mg acetazolamide is administered every other day. In one embodiment, 250 mg acetazolamide is administered twice a day. In one embodiment, 250 mg acetazolamide is administered once a day. In one embodiment, 250 mg acetazolamide is administered every other day. In one embodiment, 125 mg acetazolamide is administered twice a day. In one embodiment, 125 mg acetazolamide is administered once a day. In one embodiment, 125 mg acetazolamide is administered every other day.

In one embodiment, 25 mg of methazolamide is administered once a day. In one embodiment, 25 mg of methazolamide is administered twice a day. In one embodiment, 50 mg of methazolamide is administered once a day. In one embodiment, 50 mg of methazolamide is administered twice a day. In one embodiment, 100 mg of methazolamide is administered once a day. In one embodiment, 100 mg of methazolamide is administered twice a day.

In one embodiment, 25 mg of zonisamide is administered once a day. In one embodiment, 25 mg of zonisamide is administered twice a day. In one embodiment, 50 mg of zonisamide is administered once a day. In one embodiment, 50 mg of zonisamide is administered twice a day. In one embodiment, 100 mg of zonisamide is administered once a day. In one embodiment, 100 mg of zonisamide is administered twice a day. In one embodiment, 25 mg of zonisamide is administered once every other day. In one embodiment, 50 mg of zonisamide is administered every other day. In one embodiment, 100 mg of zonisamide is administered once a day. In one embodiment, 100 mg of zonisamide is administered every other day. In one embodiment, 100 mg of sulthiame is administered once a day. In one embodiment, 100 mg of sulthiame is administered twice a day. In one embodiment, 200 mg of sulthiame is administered once a day. In one embodiment, 200 mg of sulthiame is administered twice a day.

In one embodiment, 100 mg of bichlorfenamide is administered once a day. In one embodiment, 100 mg of bichlorfenamide is administered twice a day. In one embodiment, 200 mg of bichlorfenamide is administered once a day. In one embodiment, 200 mg of bichlorfenamide is administered twice a day.

### Instructions

In some embodiments, instructions are provided with a therapeutic combination disclosed herein providing dose level and dosing frequency of the somatostatin mimetic and a carbonic anhydrase inhibitor. Such instructions may, in some embodiments, instruct the patient to self-administer the dose of the intranasal somatostatin mimetic a certain number of times per day (or in some cases less frequently) and number of insufflations per nostril at the time of each dose administration. The instructions may, in some embodiments, instruct the patient to self-administer the dose of a carbonic anhydrase inhibitor a certain number of times per day (or in some cases less frequently). Such instructions may be provided with a kit or package comprising a certain number of unit doses of each component, or may be provided by prescription by a health care professional, or may be provided on the product label (including full prescribing information).

In some embodiments, instructions are provided for adjusting or increasing the dose of the somatostatin mimetic and/or the carbonic anhydrase inhibitor if insufficient benefit is achieved from the initial dosing regimen. Such increase can be, by way of non-limiting example, increasing the frequency of administration from once a day to twice a day of either or both components; increasing the dose by 25%, 50% or 100% of each or both components administered at the same dosing frequency. In some embodiments, both the dose level and dose frequency is increased of one or both components, following the dose adjustment instructions provided with the combination as prescribed by the health care professional.

### Packaging

In one embodiment, the carbonic anhydrase inhibitor tablet or capsules are blister packed separately, and the single doses for the nasal delivery device or a prefilled delivery device, are also blister packed separately. Together, the two separately packaged products are packaged within one package with instructions. In some embodiments, a prefilled nasal delivery device providing for multiple doses is provided in the package. In one embodiment, such packaging provides for dosing for a period of time such as but not limited to one day, one week, two weeks, three weeks, or four weeks.

### Side Effects and Assessment

As noted herein, carbonic anhydrase inhibitors exhibit an undesirable side effect profile at the dose levels and frequencies recommended for approved indications and dose levels and frequencies in clinical trials including those for IIH. The dose levels and/or dosing frequency disclosed herein in some embodiments is lower than the dose levels and/or dosing frequency of a carbonic anhydrase inhibitors for approved indications those under evaluation, and therefore should not elicit the same side effect profile, and should elicit fewer if any side effects. In some embodiments, the therapeutic combination elicits an improved side effect profile or has reduced or no reported side effects. Notwithstanding any regulatory requirement to list in instructions or prescribing information any or all known or reported side effects of the individual components of the therapeutic combination disclosed herein, such listing of such side effects is not intended in any way to negate the improved side effect profile of the therapeutic combination disclosed here, when administered following the recommended dosing regimen.

In some embodiments, treatment using topiramate in accordance with the disclosure herein provides a reduced side effect profile (e.g., fewer or reduced intensity of one or more side effects) compared with that described on the product label, using the dose levels and/or dosing regimen recommended on the product label. Side effects that are reduced or eliminated using the therapeutic combinations disclosed herein include, but are not limited to, acute myopia, secondary angle closure glaucoma, acute onset of decreased visual acuity, ocular pain, myopia, anterior chamber shallowing, ocular hyperemia (redness), increased intraocular pressure, mydriasis, anterior displacement of the lens, oligohydrosis, hyperthermia, metabolic acidosis (and its sequelae), suicidal thoughts or behavior, emerging or worsening depression, unusual change in mood or behavior, cognitive-related dysfunction (including but not limited to confusion, psychomotor slowing, difficulty with concentration/attention, difficulty with memory, speech or language problems, particularly word-finding difficulties); psychiatric/behavioral disturbances (including but not limited to depression or mood problems), somnolence or fatigue, kidney stones, hypothermia with concomitant valproic acid use, paresthesias (usually tingling in the extremities), and decreased hepatic function.

Thus, in some embodiment, side effects of topiramate such as impaired mental alertness, coordination, heat regulation or visual field effects are reduced or absent by using the therapeutic combination or following the dosing regimen disclosed herein.

In some embodiments, treatment using acetazolamide in accordance with the disclosure herein provides a reduced side effect profile (e.g., fewer or reduced intensity of one or more side effects) compared with that described on the product label, using the dose levels and/or dosing regimen recommended on the product label. Side effects that are reduced or eliminated using the therapeutic combinations disclosed herein include, but are not limited to numbness, ringing in the ears, loss of appetite, vomiting, and sleepiness.

In one embodiment, the therapeutic combination and methods of use disclosed herein using acetazolamide eliminate or have a reduced effect on weight loss. In one embodiment, the therapeutic combination and methods of use disclosed herein eliminate or have a reduced effect on paresthesias. In one embodiment, the therapeutic combination and methods of use disclosed herein eliminate or have a reduced effect on tingling in the feet. In one embodiment, the therapeutic combination and methods of use disclosed herein eliminate or have a reduced effect on dysgeusia. In one embodiment, the therapeutic combination and methods of use disclosed herein eliminate or have a reduced effect on fatigue. In one embodiment, the therapeutic combination and methods of use disclosed herein eliminate or have a reduced effect on decreased carbon dioxide level. In one embodiment, the therapeutic combination and methods of use disclosed herein eliminate or have a reduced effect on nausea. In one embodiment, the therapeutic combination and methods of use disclosed herein eliminate or have a reduced effect on, vomiting. In one embodiment, the therapeutic combination and methods of use disclosed herein eliminate or have a reduced effect on diarrhea. In one embodiment, the therapeutic combination and methods of use disclosed herein eliminate or have a reduced effect on tinnitus. In one embodiment, the therapeutic combination and methods of use disclosed herein eliminate or have a reduced effect on weight loss. In one embodiment, the therapeutic combination and methods of use disclosed herein eliminate or have a reduced effect on concentration difficulties.

In one embodiment, the therapeutic combination and methods of use disclosed herein using methazolamide eliminate or have a reduced effect on, by system: Central nervous system: Confusion, drowsiness, fatigue, flaccid paralysis, malaise, paresthesia, seizure; Dermatologic: Erythema multiforme, skin photosensitivity, skin rash, Stevens-Johnson syndrome, toxic epidermal necrolysis, urticaria; Endocrine & metabolic: Electrolyte disturbance, glycosuria, metabolic acidosis; Gastrointestinal: Decreased appetite, diarrhea, dysgeusia, melena, nausea, vomiting; Genitourinary: Crystalluria, hematuria; Hematologic & oncologic: Agranulocytosis, aplastic anemia, bone marrow depression, hemolytic anemia, immune thrombocytopenia, leukopenia, pancytopenia; Hepatic: Fulminant hepatic necrosis, hepatic insufficiency; Hypersensitivity: Anaphylaxis, hypersensitivity reaction; Ophthalmic: Myopia; Otic: Auditory disturbance, tinnitus; and Renal: Nephrolithiasis, polyuria.

In one embodiment, the therapeutic combination and methods of use disclosed herein using methazolamide eliminate or have a reduced effect on: CNS effects: May impair mental alertness and/or physical coordination; Electrolyte disturbance: Initially, potassium excretion may be increased; periodically monitor serum electrolytes and signs of hypokalemia in at risk patients; Sulfonamide (“sulfa”) allergy. Disease-related concerns include: Diabetes: Use with caution in patients with prediabetes or diabetes mellitus; may see a change in glucose control; Hepatic impairment: Use with caution in patients with hepatic impairment; may precipitate hepatic encephalopathy. Use is contraindicated in patients with marked liver impairment or cirrhosis; Respiratory disease: Use with caution in patients with respiratory disease such as emphysema or pulmonary obstruction; may precipitate or aggravate respiratory acidosis.

In one embodiment, the therapeutic combination and methods of use disclosed herein using methazolamide eliminate or have a reduced effect on nausea, vomiting, diarrhea, change in taste, lack of appetite, fatigue and loss of strength and energy.

In one embodiment, the therapeutic combination and methods of use disclosed herein using zonisamide eliminate or have a reduced effect on somnolence, anorexia, dizziness, headache, nausea, and agitation/irritability.

In one embodiment, the therapeutic combination and methods of use disclosed herein using sulthiame eliminate or have a reduced effect profile including: paresthesias in the extremities and in the face, tachypnea, hyperpnea, dyspnea, dizziness, headache, stenocardia, tachycardia, double vision, singultus, weight loss or lack of appetite. In rare cases, hallucinations, anxiety, myasthenic phenomena, lack of drive, joint pain, a grand mal state or increased seizure activity can be triggered. In individual cases, it has been suspected that sulthiame may be associated with the induction of acute renal failure, Stevens-Johnson syndrome, Lyell's syndrome, or polyneuritis. In one case, the administration of OSPOLOT led to progressive weakness of the limbs, hypersalivation, slurred speech, increasing drowsiness up to coma. The symptoms abated within hours of OSPOLOT being discontinued.

In one embodiment, the therapeutic combination and methods of use disclosed herein using bichlorphenamide eliminate or have a reduced effects among paresthesia, cognitive disorder, dysgeusia, and confusional state.

### Assessment of Efficacy and Persistence of Effect/Lack of Dropouts

In some embodiments, the efficacy of the therapeutic combination is demonstrated by any one or more characteristics such as but not limited to improvement in the disease or condition, time to observe a treatment response, reduction of symptoms of the disease or condition, longer intervals between recurrence of symptoms or sequelae of the disease or condition, persistence of symptom or sequelae-free periods, complete remission of disease, improved compliance in maintaining the dosing regimen, and reduced discontinuation or modification of the dosing regimen. Any one of more of these characteristics, optionally in combination with reduced side effects as described elsewhere, are among the benefits of the therapeutic combinations disclosed herein.

In some embodiments, the therapeutic combination disclosed herein is provided to effectively treat IIH or cluster headache in patients. Such treatment includes improvement in or reduction of symptoms and sequelae such as headaches, visual changes, increased intracranial pressure, altered vision, intracranial noise (e.g., pulse synchronous tinnitus), stiff neck, back and arm pain, pain behind the eyes, exercise intolerance and memory difficulties.

In some embodiments, the therapeutic combination is provided to effectively treat IIH or cluster headache in patients in which have not found benefit by any prior therapeutic regimen (such as but not limited to a carbonic anhydrase inhibitor). In some embodiments, the therapeutic combination is provided to effectively treat IIH or cluster headache in patients who have failed treatment by another therapeutic regimen (such as but not limited to a carbonic anhydrase inhibitor). In some embodiments, the therapeutic combination is provided to effectively treat IIH or cluster headache in patients in whom another therapeutic regimen has become less effective, not tolerated, or ineffective (such as but not limited to a carbonic anhydrase inhibitor).

In some embodiments, the therapeutic combination is provided to effectively treat IIH or cluster headache in a greater number or proportion of patients than the number or proportion of IIH or cluster headache patients in whom intranasal administration of a somatostatin mimetic alone is effective. In some embodiments, the therapeutic combination is provided to effectively treat IIH or cluster headache in patients and reduce the number or proportion of patients in which administration of a carbonic anhydrase inhibitor alone has side effect profile that may limit patient compliance. In some embodiments, the therapeutic combination is provided to effectively treat IIH or cluster headache in patients and reduce the number or proportion of patients in which administration of a carbonic anhydrase inhibitor alone at the dose that does not elicit side effect profile that may limit patient compliance, but has limited if any efficacy on symptoms of IIH or cluster headache.

The following therapeutic combinations, and methods for their administration, are embraced herein comprising octreotide and a carbonic anhydrase inhibitor.

### Abbreviations

LA: octreotide 0.1 mg/mL, Avicel CL611 2% (w/v), polysorbate 80 2% (w/v), BAC 0.02%
(w/v) in 0.01M citrate buffer pH 4.5.
LB: octreotide 1.0 mg/mL, Avicel CL611 2% (w/v), polysorbate 80 2% (w/v), BAC 0.02% (w/v) in 0.01M citrate buffer pH 4.5.
LC: octreotide 1.0 mg/mL, PEG 400 2% (w/v), polysorbate 80 2% (w/v), BAC 0.02% (w/v) in 0.01M citrate buffer pH 4.5.
LD: octreotide 0.1 mg/mL, PEG 400 2% (w/v), polysorbate 80 2% (w/v), BAC 0.02% (w/v) in 0.01M citrate buffer pH 4.5.
PA: Octreotide 0.1%, mannitol 95%, hydrogenated lecithin 5%.
PB: Octreotide 0.5%, mannitol 95%, hydrogenated lecithin 5%
PC: Octreotide 0.1%, calcium carbonate 95%, mannitol 2.5%, L-leucine 2.5%.
PD: Octreotide 0.5%, calcium carbonate 95%, mannitol 2.5%, L-leucine 2.5%.
TO: topiramate
AC: acetazolamide
LA, 100 mcg q.d.; topiramate 50 mg b.i.d.
LA, 100 mcg q.d.; topiramate 25 mg b.i.d.
LA, 100 mcg q.d.; topiramate 15 mg b.i.d.
LA, 100 mcg q.d.; topiramate 50 mg q.d.
LA, 100 mcg q.d.; topiramate 25 mg q.d.
LA, 100 mcg q.d.; topiramate 15 mg q.d.
LA, 100 mcg q.d.; acetazolamide 500 mg b.i.d.
LA, 100 mcg q.d.; acetazolamide 250 mg b.i.d.
LA, 100 mcg q.d.; acetazolamide 125 mg b.i.d.
LA, 100 mcg q.d.; acetazolamide 500 mg q.d.
LA, 100 mcg q.d.; acetazolamide 250 mg q.d.
LA, 100 mcg q.d.; acetazolamide 125 mg q.d.
LB, 100 mcg q.d.; topiramate 50 mg b.i.d.
LB, 100 mcg q.d.; topiramate 25 mg b.i.d.
LB, 100 mcg q.d.; topiramate 15 mg b.i.d.
LB, 100 mcg q.d.; topiramate 50 mg q.d.
LB, 100 mcg q.d.; topiramate 25 mg q.d.
LB, 100 mcg q.d.; topiramate 15 mg q.d.
LB, 100 mcg q.d.; acetazolamide 500 mg b.i.d.
LB, 100 mcg q.d.; acetazolamide 250 mg b.i.d.
LB, 100 mcg q.d.; acetazolamide 125 mg b.i.d.
LB, 100 mcg q.d.; acetazolamide 500 mg q.d.
LB, 100 mcg q.d.; acetazolamide 250 mg q.d.
LB, 100 mcg q.d.; acetazolamide 125 mg q.d.
LC, 100 mcg q.d.; topiramate 50 mg b.i.d.
LC, 100 mcg q.d.; topiramate 25 mg b.i.d.
LC, 100 mcg q.d.; topiramate 15 mg b.i.d.
LC, 100 mcg q.d.; topiramate 50 mg q.d.
LC, 100 mcg q.d.; topiramate 25 mg q.d.
LC, 100 mcg q.d.; topiramate 15 mg q.d.
LC, 100 mcg q.d.; acetazolamide 500 mg b.i.d.
LC, 100 mcg q.d.; acetazolamide 250 mg b.i.d.
LC, 100 mcg q.d.; acetazolamide 125 mg b.i.d.
LC, 100 mcg q.d.; acetazolamide 500 mg q.d.
LC, 100 mcg q.d.; acetazolamide 250 mg q.d.
LC, 100 mcg q.d.; acetazolamide 125 mg q.d.
LD, 100 mcg q.d.; topiramate 50 mg b.i.d.
LD, 100 mcg q.d.; topiramate 25 mg b.i.d.
LD, 100 mcg q.d.; topiramate 15 mg b.i.d.
LD, 100 mcg q.d.; topiramate 50 mg q.d.
LD, 100 mcg q.d.; topiramate 25 mg q.d.
LD, 100 mcg q.d.; topiramate 15 mg q.d.
LD, 100 mcg q.d.; acetazolamide 500 mg b.i.d.
LD, 100 mcg q.d.; acetazolamide 250 mg b.i.d.
LD, 100 mcg q.d.; acetazolamide 125 mg b.i.d.
LD, 100 mcg q.d.; acetazolamide 500 mg q.d.
LD, 100 mcg q.d.; acetazolamide 250 mg q.d.
LD, 100 mcg q.d.; acetazolamide 125 mg q.d.
PA, 100 mcg q.d.; topiramate 50 mg b.i.d.
PA, 100 mcg q.d.; topiramate 25 mg b.i.d.
PA, 100 mcg q.d.; topiramate 15 mg b.i.d.
PA, 100 mcg q.d.; topiramate 50 mg q.d.
PA, 100 mcg q.d.; topiramate 25 mg q.d.
PA, 100 mcg q.d.; topiramate 15 mg q.d.
PA, 100 mcg q.d.; acetazolamide 500 mg b.i.d.
PA, 100 mcg q.d.; acetazolamide 250 mg b.i.d.
PA, 100 mcg q.d.; acetazolamide 125 mg b.i.d.
PA, 100 mcg q.d.; acetazolamide 500 mg q.d.
PA, 100 mcg q.d.; acetazolamide 250 mg q.d.
PA, 100 mcg q.d.; acetazolamide 125 mg q.d.
PB, 100 mcg q.d.; topiramate 50 mg b.i.d.
PB, 100 mcg q.d.; topiramate 25 mg b.i.d.
PB, 100 mcg q.d.; topiramate 15 mg b.i.d.
PB, 100 mcg q.d.; topiramate 50 mg q.d.
PB, 100 mcg q.d.; topiramate 25 mg q.d.
PB, 100 mcg q.d.; topiramate 15 mg q.d.
PB, 100 mcg q.d.; acetazolamide 500 mg b.i.d.
PB, 100 mcg q.d.; acetazolamide 250 mg b.i.d.
PB, 100 mcg q.d.; acetazolamide 125 mg b.i.d.
PB, 100 mcg q.d.; acetazolamide 500 mg q.d.
PB, 100 mcg q.d.; acetazolamide 250 mg q.d.
PB, 100 mcg q.d.; acetazolamide 125 mg q.d.
PC, 100 mcg q.d.; topiramate 50 mg b.i.d.
PC, 100 mcg q.d.; topiramate 25 mg b.i.d.
PC, 100 mcg q.d.; topiramate 15 mg b.i.d.
PC, 100 mcg q.d.; topiramate 50 mg q.d.
PC, 100 mcg q.d.; topiramate 25 mg q.d.
PC, 100 mcg q.d.; topiramate 15 mg q.d.
PC, 100 mcg q.d.; acetazolamide 500 mg b.i.d.
PC, 100 mcg q.d.; acetazolamide 250 mg b.i.d.
PC, 100 mcg q.d.; acetazolamide 125 mg b.i.d.
PC, 100 mcg q.d.; acetazolamide 500 mg q.d.
PC, 100 mcg q.d.; acetazolamide 250 mg q.d.
PC, 100 mcg q.d.; acetazolamide 125 mg q.d.
PD, 100 mcg q.d.; topiramate 50 mg b.i.d.
PD, 100 mcg q.d.; topiramate 25 mg b.i.d.
PD, 100 mcg q.d.; topiramate 15 mg b.i.d.
PD, 100 mcg q.d.; topiramate 50 mg q.d.
PD, 100 mcg q.d.; topiramate 25 mg q.d.
PD, 100 mcg q.d.; topiramate 15 mg q.d.
PD, 100 mcg q.d.; acetazolamide 500 mg b.i.d.
PD, 100 mcg q.d.; acetazolamide 250 mg b.i.d.
PD, 100 mcg q.d.; acetazolamide 125 mg b.i.d.
PD, 100 mcg q.d.; acetazolamide 500 mg q.d.
PD, 100 mcg q.d.; acetazolamide 250 mg q.d.
PD, 100 mcg q.d.; acetazolamide 125 mg q.d.

As noted elsewhere herein, any one of such therapeutic combinations may be provided in a separate package within a combined package, with instructions for use, wherein each of the octreotide formulation and/or delivery device, and the carbonic anhydrase inhibitor, or both, provided in a blister pack or other suitable packaging such that the doses of each as prescribed are clearly and readily available for the patient to administer in accordance with a prescribed dosing regimen.

The following numbered embodiments, while non-limiting, are examples of certain aspects of the present disclosure.

1. A method for treating idiopathic intracranial hypertension (IIH) or cluster headache in a subject in need thereof, comprising administering to the subject a therapeutically effective combination of:
(a) an intranasal dosing regimen of a somatostatin mimetic, wherein the dosing regimen of the somatostatin mimetic provides a dose level or exposure that is less than the therapeutically effective dosing regimen when administered alone by a non-nasal route of administration; and
(b) a dosing regimen of a carbonic anhydrase inhibitor, wherein the dosing regimen of a carbonic anhydrase inhibitor provides a dose level or exposure that is less than the therapeutically effective dose when administered alone.

2. The method of embodiment 1 wherein the intranasal dosing regimen of the somatostatin mimetic is direct nose-to-brain administration.

3. The method of embodiment 1 wherein the dosing regimen of a carbonic anhydrase inhibitor elicits fewer or no side effects than that elicited by the therapeutically effective dose when administered alone.

4. The method of embodiment 1 wherein a serum area-under-the-curve from a unit dose of the intranasal amount of the somatostatin mimetic is less than the serum area-under-the-curve of an effective unit dose of the somatostatin mimetic administered by a non-intranasal route.

5. The method of embodiment 1 or 2 wherein a serum area-under-the-curve from a unit dose of the intranasal amount of the somatostatin mimetic is less than the serum area-under-the-curve from an effective unit dose of the somatostatin mimetic administered by the intranasal route for an indication other than IIH or cluster headache.

6. The method of embodiment 5 wherein the indication other than IIH or cluster headache is acromegaly or carcinoid syndrome.

7. The method of embodiment 3 or 4 wherein the non-intranasal route is subcutaneous, intramuscular, oral or intravenous.

8. The method of embodiments 1 or 2 wherein the effective intranasal amount is about equal to or less than about 100 mcg daily.

9. The method of embodiment 4 wherein the serum area-under-the-curve of the intranasal amount of the somatostatin mimetic is about equal to or less than about 50% of the serum area-under-the-curve of the somatostatin mimetic administered by a non-intranasal route.

10. The method of embodiment 4 wherein the serum area-under-the-curve of the intranasal amount of the somatostatin mimetic is about equal to or less than about 25% of the serum area-under-the-curve of the somatostatin mimetic administered by a non-intranasal route.

11. The method of embodiment 4 wherein the serum area-under-the-curve of the intranasal amount of the somatostatin mimetic is about equal to or less than about 10% of the serum area-under-the-curve of the somatostatin mimetic administered by a non-intranasal route.

12. The method of embodiment 5 wherein the serum area-under-the-curve of the intranasal amount of the somatostatin mimetic is about equal to or less than about 50% of the serum area-under-the-curve of the somatostatin mimetic administered by the intranasal route for an indication other than IIH or cluster headache.

13. The method of embodiment 5 wherein the serum area-under-the-curve of the intranasal amount of the somatostatin mimetic is about equal to or less than about 25% of the serum area-under-the-curve of the somatostatin mimetic administered by the intranasal route for an indication other than IIH or cluster headache.

14. The method of embodiment 5 wherein the serum area-under-the-curve of the intranasal amount of the somatostatin mimetic is about equal to or less than about 10% of the serum area-under-the-curve of the somatostatin mimetic administered by the intranasal route for an indication other than IIH or cluster headache.

15. The method of any one of embodiments 1-14 wherein the effective intranasal amount is administered as a single daily dose or as 2, 3 or 4 divided doses.

16. The method of any one of embodiments 1-2 wherein the effective intranasal amount is administered daily for a duration effective to treat or resolve the IIH or cluster headache in combination with the carbonic anhydrase inhibitor.

17. The method of embodiment 1 or 2, wherein the somatostatin mimetic is selected from somatostatin, octreotide, lanreotide, pasireotide, pentetreotide, or any combination thereof.

18. The method of embodiment 1 wherein the somatostatin mimetic formulated for intranasal administration comprises a powder, liquid or gel.

19. The method of embodiment 1 or 2 wherein the effective intranasal amount of somatostatin mimetic provides a minimal effective dose.

20. The method of embodiment 1 or 2 wherein treating is to reduce or prevent an acute, ongoing episode, and/or prophylactic and/or maintenance therapy to prevent future episodes.

21. The method of embodiment 1 wherein a carbonic anhydrase inhibitor is administered orally.

22. The method of embodiment 1 wherein a carbonic anhydrase inhibitor is administered daily.

23. The method of embodiment 1 wherein the therapeutically effective combination is a synergistic combination.

24. The method of embodiment 1 wherein the carbonic anhydrase inhibitor is selected from among topiramate, acetazolamide, methazolamide, zonisamide, sulthiame, bichlorphenamide, or a combination thereof.

25. The method of embodiment 1 wherein the dose level, dose frequency, or combination thereof of a carbonic anhydrase inhibitor when administered alone elicits fewer side effects than elicited by the dose level, dose frequency or combination thereof that is effective to treat approved indications.

26. The method of embodiment 1 wherein the dose level, dose frequency, or combination thereof of a carbonic anhydrase inhibitor when administered alone elicits fewer side effects than elicited by the dose level, dose frequency or combination thereof that is effective to treat IIH or cluster headache.

27. The method of embodiment 1 wherein the carbonic anhydrase inhibitor is acetazolamide and the dosing regimen is about 500 mg twice a day, about 250 mg twice a day, 125 mg twice a day, 100 mg twice a day, 50 mg twice a day, 25 mg twice a day, 20 mg twice a day, 15 mg twice a day or 10 mg twice a day.

28. The method of embodiment 1 wherein the carbonic anhydrase inhibitor is acetazolamide and the dosing regimen is about 500 mg once a day, about 250 mg once a day, 125 mg once a day, 100 mg once a day, 50 mg once a day, 25 mg once a day, 20 mg once a day, 15 mg once a day, 10 mg once a day, or 5 mg once a day.

29. The method of embodiment 1 wherein the carbonic anhydrase inhibitor is topiramate and the dosing regimen is about 50 mg twice a day, about 25 mg twice a day, about 20 mg twice a day, about 15 mg twice a day, about 10 mg twice a day, or about 5 mg twice a day.

30. The method of embodiment 1 wherein the carbonic anhydrase inhibitor is topiramate and the dosing regimen is about 50 mg once a day, about 25 mg once a day, about 20 mg once a day, about 15 mg once a day, about 10 mg once a day, or about 5 mg once a day.

31. A therapeutic combination for treatment of idiopathic intracranial hypertension (IIH) or cluster headache, comprising
(a) one or more unit doses of a somatostatin mimetic formulated for intranasal administration, wherein the dosing regimen of the somatostatin mimetic unit dose provides a dose level or exposure that is less than the therapeutically effective dosing regimen when administered alone by a non-nasal route of administration;
(b) one or more unit doses of a carbonic anhydrase inhibitor, wherein the dosing regimen of a unit dose level of a carbonic anhydrase inhibitor provides a dose or exposure that is less than the therapeutically effective dose when administered alone; and
(c) instructions for individual administration of each of the somatostatin mimetic and a carbonic anhydrase inhibitor in accordance with an effective dosing regimen.

32. The therapeutic combination of embodiment 31 wherein the intranasal dosing regimen is direct nose-to-brain administration.

33. The therapeutic combination of embodiment 31 or 32 wherein the dosing regimen of a carbonic anhydrase inhibitor elicits fewer or no side effects than that elicited by the therapeutically effective dose when administered alone.

34. The therapeutic combination of embodiment 31 or 32 wherein a serum area-under-the-curve of a unit dose of the somatostatin mimetic administered intranasally is less than the serum area-under-the-curve of the unit dose of the somatostatin mimetic administered by a non-intranasal route.

35. The therapeutic combination of embodiment 32 or 33 wherein the non-intranasal route is subcutaneous, intramuscular oral or intravenous.

36. The therapeutic combination of embodiment 31 or 32 wherein a unit dose of the somatostatin mimetic formulated for intranasal administration is about equal to or less than about 100 mcg.

37. The therapeutic combination of embodiment 31 or 32 wherein a unit dose of the somatostatin mimetic formulated for intranasal administration is about equal to or less than about 50 mcg.

38. The therapeutic combination of embodiment 31 or 32 wherein a unit dose of the somatostatin mimetic formulated for intranasal administration is about equal to or less than about 10 mcg.

39. The therapeutic combination of embodiment 31 or 32 wherein a unit dose of the somatostatin mimetic formulated for intranasal administration is about equal to or less than about 5 mcg.

40. The therapeutic combination of embodiment 31 or 32 wherein a unit dose of the somatostatin mimetic formulated for intranasal administration is equal to about 1 mcg.

41. The therapeutic combination of embodiment 34 wherein the serum area-under-the-curve of a unit dose of the somatostatin mimetic administered intranasally is about equal to or less than about 50% of the serum area-under-the-curve of a unit dose of the somatostatin mimetic administered by a non-intranasal route.

42. The therapeutic combination of embodiment 34 wherein the serum area-under-the-curve of a unit dose of the somatostatin mimetic administered intranasally is about equal to or less than about 25% of the serum area-under-the-curve of a unit dose of the somatostatin mimetic administered by a non-intranasal route.

43. The therapeutic combination of embodiment 34 wherein the serum area-under-the-curve of a unit dose of the somatostatin mimetic administered intranasally is about equal to or less than about 10% of the serum area-under-the-curve of a unit dose of the somatostatin mimetic administered by a non-intranasal route.

44. The therapeutic combination of embodiment 34 wherein a serum area-under-the-curve from a unit dose of the intranasal amount of the somatostatin mimetic is less than the serum area-under-the-curve from an effective single dose of the somatostatin mimetic administered by the intranasal route for an indication other than IIH or cluster headache.

45. The therapeutic combination of embodiment 44 wherein the indication other than IIH or cluster headache is acromegaly or carcinoid syndrome.

46. The therapeutic combination of embodiment 31 or 32 wherein the unit dose is labeled for administration in a single daily dose or as 2, 3 or 4 divided doses.

47. The therapeutic combination of embodiment 31 or 32 wherein the unit dose is labeled for daily administration for a duration effective to treat or resolve the IIH or cluster headache.

48. The therapeutic combination of embodiment 31 or 32 wherein treatment is to reduce or prevent an acute, ongoing episode, and/or prophylactic and/or maintenance therapy to prevent future episodes.

49. The therapeutic combination of embodiment 31 wherein the somatostatin mimetic is selected from somatostatin, octreotide, lanreotide, pasireotide, pentetreotide, or any combination thereof.

50. The therapeutic combination of embodiment 31 wherein the somatostatin mimetic formulated for intranasal administration comprises a powder, liquid or gel.

51. The therapeutic combination of embodiment 31 or 32 wherein the unit dose is a minimal effective dose.

52. The therapeutic combination of embodiment 31 or 32 wherein a carbonic anhydrase inhibitor is administered orally.

53. The therapeutic combination of embodiment 31 or 32 wherein a carbonic anhydrase inhibitor is administered daily.

54. The therapeutic combination of embodiment 31 or 32 wherein the dose level, dose frequency, or combination thereof of a carbonic anhydrase inhibitor is lower than effective to treat approved indications.

55. The therapeutic combination of embodiment 31 or 32 wherein the carbonic anhydrase inhibitor is selected from among topiramate, acetazolamide, methazolamide, zonisamide, sulthiame, bichlorphenamide, or a combination thereof.

56. The therapeutic combination of embodiment 31 or 32 wherein the carbonic anhydrase inhibitor is acetazolamide and the dosing regimen is equal to or less than about 500 mg once a day or twice a day.

57. The therapeutic combination of embodiment 56 wherein the dosing regimen is equal to or less than about 250 mg once a day or twice a day.

58. The therapeutic combination of embodiment 57 wherein the dosing regimen is equal to or less than about 125 mg twice a day.

59. The therapeutic combination of embodiment 31 or 32 wherein the dosing regimen of topiramate is equal to or less than about 50 mg once a day or twice a day.

60. The therapeutic combination of embodiment 31 or 32 which is a synergistic combination.

61. The therapeutic combination of embodiment 31 or 32 wherein the dose level, dose frequency, or combination thereof of a carbonic anhydrase inhibitor when administered alone in accordance with the instructions elicits fewer side effects than elicited by the dose level, dose frequency or combination thereof that is effective to treat approved indications.

62. The therapeutic combination of embodiment 31 or 32 wherein the dose level, dose frequency, or combination thereof of a carbonic anhydrase inhibitor when administered alone in accordance with the instructions elicits fewer side effects than elicited by the dose level, dose frequency or combination thereof that is effective to treat IIH or cluster headache.

63. A therapeutic combination for treatment of idiopathic intracranial hypertension (IIH) or cluster headache, comprising
(a) one or more unit doses of a somatostatin mimetic formulated for intranasal administration;
(b) one or more unit doses of a carbonic anhydrase inhibitor; and
(c) instructions for individual administration of each of the somatostatin mimetic and a carbonic anhydrase inhibitor in accordance with an effective dosing regimen;
wherein the unit dose of a carbonic anhydrase inhibitor administered in accordance with the instructions elicits fewer side effects than the unit dose of a carbonic anhydrase inhibitor as administered alone for treatment of approved indications.

64. A therapeutic combination for treatment of idiopathic intracranial hypertension (IIH) or cluster headache, comprising
(a) one or more unit doses of a somatostatin mimetic formulated for intranasal administration;
(b) one or more unit doses of a carbonic anhydrase inhibitor; and
(c) instructions for individual administration of each of the somatostatin mimetic and a carbonic anhydrase inhibitor in accordance with an effective dosing regimen;
wherein the unit dose of a carbonic anhydrase inhibitor administered in accordance with the instructions elicits fewer side effects than the unit dose of a carbonic anhydrase inhibitor as administered alone for treatment of idiopathic intracranial hypertension or cluster headache.

### EXAMPLES

The following examples are put forth so as to provide persons having ordinary skill in the art with a complete disclosure and description of how to make and use the disclosed embodiments, and are not intended to limit the scope of as intended herein.

### Example 1. Treatment of Idiopathic Intracranial Hypertension

A patient suffering from idiopathic intracranial hypertension is started on a course of treatment with an intranasal formulation of octreotide acetate delivered using a nose-to-brain delivery formulation and device, and oral dosing with low dose acetazolamide. Within days from the initiation of daily dosing, the patient experiences relief from headache. Intracranial pressure is also found to be reduced. No side effects are associated with the administration.

### Example 2. Treatment of Cluster Headache

A patient suffering from cluster headaches without relief from any therapy during or between episodes is started on a course of treatment with an intranasal formulation of octreotide acetate delivered using a nose-to-brain delivery device, and low dose oral topiramate. Treatment is started at the onset of a headache cycle. Within one week from the initiation of daily dosing, the patient experiences a reduction in frequency of headaches which breaks the attack cycle. The patient is maintained on a less frequent dosing schedule which prevents recrudescence.

### Example 3. Treatment of Idiopathic Intracranial Hypertension

A patient suffering from idiopathic intracranial hypertension has failed on carbonic anhydrase inhibitor therapy following a dosing regimen prescribed for approved indications. The patient is started on a course of treatment of the combination of an intranasal formulation of octreotide acetate delivered using a nose-to-brain delivery formulation and device, and oral dosing with low dose topiramate. Within days from the initiation of daily dosing, the patient experiences relief from headache. Intracranial pressure is also found to be reduced. No side effects are associated with the administration.

### Example 4. Treatment of Cluster Headache

A patient suffering from cluster headaches is without relief from carbonic anhydrase inhibitor therapy following a dosing regimen prescribed for approved indications. The patient is started on a course of treatment of a combination of an intranasal formulation of octreotide acetate delivered using a nose-to-brain delivery device, and low dose oral acetazolamide. Treatment is started at the onset of a headache cycle. Within one week from the initiation of daily dosing, the patient experiences a reduction in frequency of headaches which breaks the attack cycle. The patient is maintained on a less frequent dosing schedule which prevents recrudescence.

### Example 5. Liquid Formulations of Octreotide for Nose-to-brain Delivery

Liquid formulations of octreotide acetate were prepared for evaluation of droplet size distribution (DSD), spray content uniformity (SCU), pH, osmolality and viscosity, in order to identify a suitable formulation that can be used for nose-to-brain delivery for effective treatment of IIH or cluster headache, in combination with a carbonic anhydrase inhibitor. First, a suitable HPLC system was developed to quantify octreotide. The following table shows the materials being used for this and the ensuing examples.

| Material | Part | Batch | Supplier |
|---|---|---|---|
| Octreotide | HY- | Lot# | MedChemExpress |
| Acetate salt | 17365/CS- | 116521 | |
| | 0944 | | |
| UDSp Device | Ball | 002982503A | Aptar Pharma |
| (powder) | Container Sub | 0027722920 | |
| | Stem Sub | 0028929870 | |
| | Actuator UDSP | 0028928070 | |
| VP7-232NE | Pump | NA | Aptar Pharma |
| Devices | TP27.0 mm | | |
| (liquid) | Bottle | NA | |
| | 20 mL | | |
| | HDPE | | |
| PBS | Excipients | SLCJ5627 | Sigma |
| PEG400 | | 1882895 | Fisher |
| Mannitol | | 1849103 | Fisher |
| Avicel** | | D1936C | DuPont |
| Lecithin*** | | A0414102 | Acros |
| Tween 80 | | BCBW9985 | Sigma |
| Benzalkonium | | BCCD5323 | Sigma |
| chloride (BAC) | | | |

*NA: not applicable.
**Avicel CL-611 is a blend of microcrystalline cellulose and 11.3-18.8% sodium carboxymethycellulose
***or LIPOID S 80, Batch No. 29232000, soybean phospholipids containing 75% phosphatidylcholine

A liquid formulation of octreotide was developed for delivery using a spray device, which has a delivered dose of 10 mcg or 100 mcg in 100 mcl (microliters). Octreotide formulations comprising 0.1 mg/mL octreotide were prepared for the 10 mcg dose in 100 mcl delivered volume, and formulations comprising 1.0 mg/mL octreotide for the 100 mcg dose in 100 mcl delivered volume.

The following liquid formulation compositions were prepared for testing in the analyses described, after establishing an HPLC-based analytical method described further below.
Octreotide Formulation 1—Octreotide 0.1 mg/mL, Avicel CL611 2% (w/v), polysorbate 80 2% (w/v), BAC 0.02% (w/v) in 0.01M citrate buffer pH 4.5
Octreotide Formulation 2—Octreotide 1.0 mg/mL, Avicel CL611 2% (w/v), polysorbate 80 2% (w/v), BAC 0.02% (w/v) in 0.01M citrate buffer pH 4.5
Octreotide Formulation 3—Octreotide 1.0 mg/mL, PEG 400 2% (w/v), polysorbate 80 2% (w/v), BAC 0.02% (w/v) in 0.01M citrate buffer pH 4.5
Octreotide Formulation 4—Octreotide 0.1 mg/mL, PEG 400 2% (w/v), polysorbate 80 2% (w/v), BAC 0.02% (w/v) in 0.01M citrate buffer pH 4.5.

### Example 6. Characterization of Liquid Formulations of Octreotide for Nose-to-Brain Delivery

HPLC METHOD. The compendial PhEur HPLC method was used (EP Reference: 01/2020:2414). An Agilent 1260 system with either a MWD or DAD detector is used via Reverse Phase HPLC with a YMC-Pack-ODS-AQ to separate Octreotide via gradient elution. Mobile phases of tetramethyl ammonium hydroxide in different concentrations of water (90% H₂O:10% ACN and 40% H₂O:60% ACN) pH2.0 with a gradient elution at a flow rate of 1.8 mL/min was used at a column temperature of 40° C. with an injection volume of 20 μL. UV absorbance was measured at a wavelength of 210 nm over the 50 minute run time. The HPLC parameters are summarized below.

| Parameter | Value to set up | | |
|---|---|---|---|
| HPLC System and | Agilent 1260 system, MWD or DAD | | |
| detector | detector | | |
| Phase | Reverse Phase | | |
| Column | YMC-Pack ODS-AQ, 250 mm × 4.6 mm, | | |
| | 5 μm | | |
| Mobile phase A | Tetramethyl ammonium hydroxide in water | | |
| | for chromatography pH 2.0 900:100 (v/v) | | |
| | acetonitrile; | | |
| Mobile phase B | Tetramethyl ammonium hydroxide in water | | |
| | for chromatography pH 2.0 400:600 (v/v) | | |
| | acetonitrile; | | |
| Flow rate (mL/min) | 1.8 | | |
| Detection Wavelength (nm) | 210 | | |
| Column temperature (° C.) | 40 | | |
| Autosampler temperature | 2-8° C. | | |
| (° C.) | | | |
| Injection volume (μL) | 20 | | |
| Total run time (min) | 50 | | |
| | Time (mins) | % A | % B |
| Gradient elution | 0 | 90 | 10 |
| | 15 | 80 | 20 |
| | 40 | 55 | 45 |
| | 45 | 30 | 70 |
| | 50 | 90 | 10 |

| | |
|---|---|
| Washing Procedure | H2O milliQ 95:5 MeOH |
| | 20 mins 100% ACN |
| | 20 mins |
| | H2O milliQ 50:50 ACN 20 mins |

ACN = acetonitrile

Solution Preparation. All solutions and/or raw active storage, please ensure it is in an airtight container, protected from light, at a temperature of 2° C. to 8° C.

Diluent Preparation. Mix 10 volumes of acetonitrile and 90 volumes of water, then adjust to pH 3.5 with acetic acid. Expiry: 7 days

Mobile Phase A Preparation. Dissolve 4.5 g of tetramethyl ammonium hydroxide in 800 mL of water for chromatography and adjust to pH 2.0 with phosphoric acid; dilute to 900 mL with water for chromatography and add 100 mL of acetonitrile. Expiry: 5 days

Mobile Phase B Preparation. Dissolve 4.5 g of tetramethyl ammonium hydroxide in 300 mL of water for chromatography and adjust to pH 2.0 with phosphoric acid; dilute to 400 mL with water for chromatography and add 600 mL of acetonitrile. Expiry: 5 days

Reference Stock Solution Preparation. Dissolve the contents of a vial of Octreotide CRS in the solvent mixture to obtain a concentration of 1.0 mg/mL.

Linearity, Standard Agreement and System Precision. System suitability was tested to ensure the method was fit for purpose and identified the quantification range. Serial dilutions from stocks solutions were prepared ranging from 1000.00 μg/mL to 0.05 μg/mL in diluent 90% H2O:10% acetonitrile at pH 3.5. Samples prepared above were analyzed by HPLC.

The percentage content of octreotide (C49H66N10O10S2) was calculated taking into account the assigned content of C49H66N10O10S2 in Octreotide CRS (European Pharmacopeia octreotide reference standard).

System suitability assessment was as follows:

| Parameter | Acceptance Criteria |
|---|---|
| Limit of | The Signal to Noise (S/N) ratio for the LOQ |
| Quantification (LOQ) | should be NLT 10. |
| Limit of Detection | Signal to Noise (S/N) ratio for the LOD should be |
| (LOD) | NLT 3. |
| Linearity | The correlation co-efficient should be NLT 0.99. |
| Standard Verification | Between 90-110% |
| Working precision | % RSD NMT 2.0% |
| (Initial 6 WSA | |
| injections) | |
| Specificity | PASS if Blank, diluent, Matrix Ingredients do not |
| | interfere with the main peak. |
| Tailing factor | NMT 2.0 |

Results. The system specificity was tested against the blank diluent and the excipients and no interference was seen with the main peak. Octreotide could be identified at a retention time of 20.8 minutes. The standard verification (98.5%) was also well within the required range, as was the working standard precision (0.1% RSD). The linearity showed a R 2 value of 0.9999, and the limits of quantification and detection had signal to noise ratios of 17.4 and 6.0, respectively. Peak tailing factor was around 1.4 which was below the criteria set of 2.0.

To ensure that the addition of formulation excipients do not interfere with the quantification of the API, samples of excipients were prepared in the standard diluent at the concentration of lmg/mL. These were run alongside the usual system suitability sequence using the same HPLC method used for the linearity testing. There was no interference from the diluent. None of the excipients tested showed any interference. Assay results were found to be within the target for all 4 formulations (i.e. Assay NLT 95% and NMT 105%).

### Droplet Size Distribution (DSD)—Method Development

Droplet size measurement of the aerosol emitted from the selected pumps was carried out employing Malvern Spraytec (Malvern Panalytical Ltd, UK) in an open bench configuration. The nasal spray was actuated at 3 and 6 cm from the laser in a carefully defined position with an extraction hood on top to ensure safety of the analyst as well as ensure one single crossing of the laser beam by the spray emitted after actuation. Data collected was analyzed in terms of transmittance, volume diameters of the 10th, 50th and 90th percentile of the Gaussian distribution and width of the droplets distribution obtained (span). A Malvern Mastersizer may also be used to analyze DSD.

Analysis was performed as n=6 for each of the four formulations employing two different analysts (n=3 per analyst) in order to reduce manual actuation variability.

The repeatability of the method was assessed from the standard deviation of three replicate measurements performed by two analysts. The combined variability for both analyst was within the specified limits in in the USP <429>, for all measurements except the Dv,10 for formulation 4 at 6 cm. Formulation 2 tested at 3 cm for one analyst was also slightly higher than the required criteria, in this instance the combined analysis of both analysts was within the 10% criteria.

All formulations showed comparable droplet size distribution (DSD) 15-70 μm, which showed to be suitable for nasal delivery. Particularly, a median droplet size of 30-40 μm was reported to successfully deposit in the nasal cavity (Sangolkar et al., 2012. Particle size determination of nasal drug delivery system: A review. International Journal of Pharmaceutical Sciences Review and Research 17(1), no. 14, 66-73; Trows et al., Analytical Challenges and Regulatory Requirements for Nasal Drug Products in Europe and the U.S., 2014 Apr. 11; 6(2):195-219). Bigger droplets (higher than 120 μm) would deposit at the front of the nose, while finer particles may penetrate further into the body (lungs), these last two are deposition patterns which has to be avoided for this formulation development.

### Spray Content Uniformity (SCU)—Method Development

A suitable method for the quantification of the amount of peptide emitted at every spray from the four formulations metered dose pumps coupled with nasal actuator was developed. This method was then employed to determine the SCU of the different formulations manufactured into a suitable collection container. The SCU test was performed at the beginning life stage of the liquid products.

Analysis was performed for n=10 sprays for each liquid formulation. Shot weight was recorded as well.

The Avicel formulations at 0.1 and 1.0 mg/mL, with a target delivered dose of 10.00 and 100.00 mcg, respectively, showed a SCU mean of 10.41 mcg+/−0.13 mcg, % RSD 1 and % of TDD 104; and a mean of 100.08 mcg+/−0.93 mcg, % RSD 1 and % of TDD 100, respectively.

The PEG 400 formulations at 1.0 and 0.1 mg/mL, with a target delivered dose of 100.00 and 10.00 mcg, respectively, showed a SCU mean of 98.83+/−1.31 mcg, % RSD 1 and % of TDD 99; and a mean of 9.67+/−0.14 mcg, % RSD 1 and % of TDD 97, respectively.

All formulations presented a consistent delivered dose from the Aptar pump, emitting the target amount of API for both formulation strengths. These results were in line with the acceptance criteria from the USP/EP.

pH.

pH value was measured for the liquid formulations developed in order to determine if it is suitable for nasal products and for the peptide stability. Three measurements were carried out for each liquid formulation. The pH values collected for the final formulations were as expected (target pH 4.5), suitable for nasal delivery (Dhakar et al., 2011, A review on factors affecting the design of nasal drug delivery system, International Journal of Drug Delivery 3(2), 194-208), as shown in the table below.

| pH Results | Average |
|---|---|
| Formulation 1 | 4.51 |
| Formulation 2 | 4.51 |
| Formulation 3 | 4.52 |
| Formulation 4 | 4.49 |

Osmolality. Osmolality was checked in triplicate for each liquid formulation by using a 16M Löser freezing point osmometer (Loser Messtachnik, DE). 25 μL of material was pipetted into the system sample holder for the analysis.

| Osmometry Results (mOsm) | Average |
|---|---|
| Formulation 1 | 31.33 |
| Formulation 2 | 32.67 |
| Formulation 3 | 83.67 |
| Formulation 4 | 80.00 |

Osmolality was lower than the optimal osmolality range for nasal formulations, 290-500 mOsm/kg. These low osmolality values were particularly visible with the Avicel containing formulations. The target of 290 mOsm will be obtainable with the addition of tonicity agents such as mannitol, dextrose, sodium chloride, sorbitol of fructose.

### Viscosity.

The viscosity of the formulation was measured using Kinexus Ultra+(Malvern Panalytical, UK). The following data output displayed: viscosity average and standard deviation. Viscosity of the nasal spray has an impact on spray characteristics such as droplet size and spray geometry and the nasal deposition. It was reported that nasal formulations with low viscosity tend to deposit distal to the nares compared to viscous formulations (Guo Y, 2005, The effect of formulation variables and breathing patterns on the site of nasal deposition in an anatomically correct model, Pharm Res. 2005 November; 22(11):1871-8; Sosnowski T R, 2020, Impact of physicochemical properties of nasal spray products on drug deposition and transport in the pediatric nasal cavity model, Int J Pharm. 2020 Jan. 25; 574:118911). Besides, it was found that the higher viscosity is associated with larger droplet size and narrower plume angle, leading to smaller spray area (Kundoor V, 2011, Effect of formulation- and administration-related variables on deposition pattern of nasal spray pumps evaluated using a nasal cast Pharm Res. 2011 August; 28(8):1895-904; Pu Y, 2014, A comparison of the deposition patterns of different nasal spray formulations using a nasal cast, Aerosol Science and Technology, 48:9, 930-938). Three samples for each liquid formulation were analyzed for viscosity.

The power law model was fitted on the viscometry data in order to calculate the flow consistency index, k, and the power law index, n. Tangent analysis was applied to the oscillation amplitude data to calculate the cross-over point from the Linear Viscoelatic region (LVR) to the flow region, which is being used to measure of yield stress. Equilibrium flow curves (shear viscosity vs shear rate) and plots from the oscillation amplitude sweep experiments were prepared.

The power law fit describes the behavior of a non-Newtonian fluid for the formulations comprising Avicel. It was observed that ri was less than one and the power law predicts that the effective viscosity would decrease with increasing shear rate (shear thinning profile). This is the typical behavior of formulations comprising Avicel [Rudra-raju and Wyandt, 2005, Rheological characterization of Microcrystalline Cellulose/Sodiumcarboxymethyl cellulose hydrogels using a controlled stress rheometer: part I, Int J Pharm. 2005 Mar. 23; 292(1-2):53-61]. In contrast, the formulations comprising PEG400 showed a higher variability in the viscosity collected at low shear rates and a Newtonian behavior at higher shear rate.

| Viscosity | | Standard | | Standard |
|---|---|---|---|---|
| Results | Average η | Deviation η | Average k1 | Deviation k1 |
| Formulation 1 | 0.51 | 0.04 | 0.09 | 0.02 |
| Formulation 2 | 0.59 | 0.01 | 0.07 | 0.00 |
| Formulation 3 | 0.92 | 0.06 | 0.00 | 0.00 |
| Formulation 4 | 0.97 | 0.04 | 0.00 | 0.00 |

### Example 7. Powder Formulations of Octreotide for Nose-to-Brain Delivery

A powder formulation of octreotide was developed for delivery using the Aptar UDSp device, which has a recommended fill weight of 10-80 mg. Octreotide formulations comprising 0.1% octreotide were prepared for a 10-80 mcg delivered dose based on a 10-80 mg dispensed weight, and a formulation comprising 0.5% octreotide for a 50-250 mcg delivered dose based on a 10-50 mg dispensed weight.

Method development for powder formulations. Eight different powder formulations—#M1, #M2, #M3, #M4, #C1, #C2, #C3 and #C4— were manufactured by spray drying, based on the following four octreotide compositions (all w/w):
Octreotide 0.1%, mannitol 95%, hydrogenated lecithin 5%.
Octreotide 0.5%, mannitol 95%, hydrogenated lecithin 5%.
Octreotide 0.1%, calcium carbonate 95%, mannitol 2.5%, L-leucine 2.5%
Octreotide 0.5%, calcium carbonate 95%, mannitol 2.5%, L-leucine 2.5%

Formulation code #s: M, mannitol; C, calcium carbonate; 1 or 2, 0.1% octreotide; 3 or 4; 0.5% octreotide; 1 or 3: inlet temperature 100° C.; 2 or 4, inlet temperature 120° C.

The parameters used for spray drying were a feed solution concentration 2% (w/v; total of octreotide and excipients, in water), the Inlet Temperature was either 100 or 120° C., 100% Aspiration and a Flow rate of 600 L/h. The yield of the mannitol formulation was >70% and the calcium carbonate formulation was >50%. Formulations #C1, #C2, #M1 and #M2 had an octreotide concentration of 0.1% (w/w) and formulations #C3, #C4, #M3 and #M4 were 0.5% (w/w). During the drug loading preparations the 0.1 and 0.5% values of the drug weight were subtracted from the mannitol weight, so in effect values of either 94.5 or 94.95% of mannitol were used in #M formulations and 2.45 or 2.49% of mannitol was used in the #C formulations.

The table below displays some representative details of these formulations such as composition, conditions in which the powders were spray dried, yield, residual water content and particle size distribution (PSD).

For powders M1, M2, M3 and M4 the PSD values are relative to the spray dried powder before agglomeration. Powders MI to M4 were agglomerated by placing the powder on the top of a stack of two sieves with nominal apertures of 600 mm and 106 mm, respectively (10 cm diameter sieves, Endecotts Ltd., London, UK); the final collector was added to the stack. The sieve stack was closed with the glass cover and vibrated for 5 min on a laboratory sieve shaker (amplitude 3; Analysette 3 Fritz model, Fritsch GMBH, Germany) Agglomerates retained between 600 mm and 106 mm were collected. The non-agglomerated powder in the collector was reprocessed, and the large agglomerates on the first sieve were crushed. The entire process was repeated twice. The yield of the agglomeration process was higher than 90% for all the powders.

After several reanalysis, water residual content for powders C1, C2, C3 and C4 was not detectable by TGA. Dv values represent a sphere of the same volume, and percentile of cumulative distribution. Data on the formulations are provided in the following table.

### Octreotide Powder Formulation Characterization.

| | | | | Spray Dryer | | | | | | |
|---|---|---|---|---|---|---|---|---|---|---|
| | | | | Conditions | | Spray | Residual | | | |
| | | | Theoretical | Inlet | Feed | Dryer | Water | Dv | Dv | Dv |
| Sample | | Weight | OA Conc. | Temperature | Rate | Yield | Content | (10) | (50) | (90) |
| Name | Composition | (g) | (w/w %) | (° C.) | (ml/mi | (%) | (% w/w) | (μm) | (μm) | (μm) |
| Powder | Mannitol | 5.04 | 0.1 | 100 | 5 | 63.14 | 0.14 ± | 0.53 ± | 2.73 ± | 6.63 ± |
| #M1 | Lecithin | | | | | | 0.01 | 0.01 | 0.08 | 0.08 |
| Powder | Mannitol | 5.08 | 0.1 | 120 | 5 | 69.20 | 0.11 ± | 0.57 ± | 2.37 ± | 5.85 ± |
| #M2 | Lecithin | | | | | | 0.01 | 0.00 | 0.01 | 0.03 |
| Powder | Mannitol | 5.12 | 0.5 | 100 | 5 | 61.22 | 0.23 ± | 0.42 ± | 2.46 ± | 6.38 ± |
| #M3 | Lecithin | | | | | | 0.00 | 0.00 | 0.03 | 0.26 |
| Powder | Mannitol | 4.14 | 0.5 | 120 | 5 | 42.88 | 0.11 ± | 0.68 ± | 2.01 ± | 6.15 ± |
| #M4 | Lecithin | | | | | | 0.01 | 0.01 | 0.01 | 0.15 |
| Powder | CaCO3 | 5.92 | 0.1 | 100 | 5 | 58.84 | 0 | 3.29 ± | 14.40 ± | 26.20 ± |
| #C1 | Mannitol L- | | | | | | | 1.60 | 0.47 | 0.13 |
| | Leucine | | | | | | | | | |
| Powder | CaCO3 | 5.30 | 0.1 | 120 | 5 | 51.25 | 0 | 4.55 ± | 14.50 ± | 26.00 ± |
| #C2 | Mannitol L- | | | | | | | 0.30 | 0.11 | 0.20 |
| | Leucine | | | | | | | | | |
| Powder | CaCO3 | 5.49 | 0.5 | 100 | 5 | 58.68 | 0 | 3.69 ± | 14.20 ± | 25.40 ± |
| #C3 | Mannitol L- | | | | | | | 1.03 | 0.16 | 0.12 |
| | Leucine | | | | | | | | | |
| Powder | CaCO3 | 5.89 | 0.5 | 120 | 5 | 59.66 | 0 | 5.37 ± | 14.10 ± | 25.10 ± |
| #C4 | Mannitol L- | | | | | | | 0.44 | 0.25 | 0.25 |
| | Leucine | | | | | | | | | |

### Example 8. Further Characterization of Powder Formulations of Octreotide for Nose-to-Brain Delivery

Spray Content Uniformity. Calcium carbonate based low dose formulations have an expected content of 0.1% (w/w) and a target delivered dose of 50 mcg. Formulation C1 had a mean SCU of 16.60 (SD 1.10; % RSD 6.65, % of TDD 33.20), and Formulation C2 40.24 (SD 1.76, % RSD 4.38, % of TDD 80.47). Calcium carbonate based high dose formulations have an expected content of 0.5% (w/w) and a target delivered dose of 250 mcg. High dose Formulation C3 had a mean SCU of 196.35 (SD 16.33, % RSD 8.32, % of TDD 78.54), and Formulation C4 has a SCU of 173.96 (SD 17.75, % RSD 10.20, % of TDD 69.58). The recoveries from the single 25 mg shots of either concentration were recovered from 10 mL volumetric, filtered through a nylon 0.2 μm filter into a HPLC vial for testing. The #C1 showed the lowest recovery obtained from all eight formulations at ˜33% of the TTD.

The #C2 formulation had a better recovery at #80% of the TTD, and was more consistent with both the higher doses of #C3 ˜79%, and #C4 ˜70%.

The data was consistent within batches as displayed by the relatively low % RSD values that ranged from ˜4-10%.

These data sets align with the assay results obtained by HPLC, which indicated that the active was lost in other regions of the spray drier hence the low drug loading.

Mannitol based low dose formulations have an expected content of 0.1% (w/w) and a target delivered dose of 50 mcg. The mannitol-based low dose formulations Formulation M1 has a mean SCU of 50.95 (SD 0.89; % RSD 1.74, % of TDD 101.89), and Formulation M2 51.22 (SD 1.36, % RSD 2.65, % of TDD 102.44). Mannitol based high dose formulations have an expected content of 0.5% (w/w) and a target delivered dose of 250 mcg. High dose Formulation M3 has a mean SCU of 251.43 (SD 5.50, % RSD 2.19, % of TDD 100.57), and Formulation M4 has a SCU of 246.81 (SD 3.74, % RSD 1.52, % of TDD 98.72).

As before, the recoveries from the single 25 mg shots of either concentration were recovered from 10 mL volumetric, filtered through a nylon 0.2 μm filter into a HPLC vial for testing. Unlike the #C formulations, there was less variation between the #M formulations which ranged from 98% (#M4) to 102% (#M2) of the TTD.

The data was very consistent within batches, even more so that the #C formulations, as displayed by the relatively low % RSD values that ranged from ˜1.5-2.7%.

Droplet Size Distribution.

The DSD for the calcium carbonate formulations are show below.

| | | | |
|---|---|---|---|
| Analyst 1 (JS) | D10 | D50 | D90 |

Formulation #C1 - Distance 3 cm

| | | | |
|---|---|---|---|
| Average | 11.91 | 19.14 | 30.38 |
| STD | 0.27 | 0.39 | 0.61 |
| RSD % | 2.29 | 2.05 | 2.00 |

| | |
|---|---|
| Span | 0.97 |

Formulation #C2 - Distance 3 cm

| | | | |
|---|---|---|---|
| Average | 13.50 | 21.57 | 34.03 |
| STD | 0.59 | 0.71 | 0.80 |
| RSD % | 4.37 | 3.27 | 2.34 |

| | |
|---|---|
| Span | 0.95 |

Formulation #C3 - Distance 3 cm

| | | | |
|---|---|---|---|
| Average | 11.66 | 18.38 | 28.62 |
| STD | 0.64 | 0.49 | 0.07 |
| RSD % | 5.52 | 2.66 | 0.23 |

| | |
|---|---|
| Span | 0.92 |

Formulation #C4 - Distance 3 cm

| | | | |
|---|---|---|---|
| Average | 12.21 | 18.67 | 28.29 |
| STD | 0.23 | 0.42 | 0.79 |
| RSD % | 1.92 | 2.27 | 2.80 |

| | |
|---|---|
| Span | 0.86 |

In general the results from these formulations produced particle diameters of a good value for a nasal spray with D50 values ranging from ˜18 μm (#C3) to ˜22 μm (#C2) indicating both consistency within each batch and between all #C batches.

### Powder Formulation Method Development.

The assay recovery for all powder octreotide formulations is shown in the following table.

| Assay Results | Average | % | Target |
|---|---|---|---|
| (Concentration μg/mL) | conc. | Assay | mass (μg). |
| C1 | 32.20 | 32.20 | 100 |
| C2 | 76.60 | 76.60 | |
| C3 | 423.50 | 84.70 | 500 |
| C4 | 397.51 | 79.50 | |
| M1 | 98.06 | 98.06 | 100 |
| M2 | 96.67 | 96.67 | |
| M3 | 468.53 | 93.71 | 500 |
| M4 | 458.27 | 91.65 | |

The assay results mirror the observations seen in the SCU data for all of the formulations. Both the low and high dose formulations performed well with the mannitol based formulations, with the lower dose showing the highest levels of recovery 97-99%. The calcium carbonate based formulation showed the same pattern as the SCU data with very similar values of recovery as well.

Any of the foregoing formulations of octreotide can be used in a therapeutic combination with a carbonic anhydrase inhibitor, for the purposes disclosed herein.

### Example 9. Treatment of Idiopathic Intracranial Hypertension Using Octreotide Nose-to-Brain Liquid Formulation

A patient suffering from idiopathic intracranial hypertension is started on a course of treatment with an intranasal formulation of octreotide acetate delivered using a nose-to-brain delivery as described herein, and low dose acetazolamide. For the octreotide, a liquid formulation comprising octreotide 0.1 mg/mL, Avicel CL611 2% (w/v), polysorbate 80 2% (w/v), BAC 0.02% (w/v) in 0.01M citrate buffer pH 4.5 is used; the tonicity is increased with NaCl to about 290 mOsm/kg. Delivery is using a VP7-232NE device (Aptar Pharma). The device delivers 100 mcl per pump. The dose of octreotide is 10 mcg per pump. Acetazolamide is administered at 125 mg once a day, orally. The patient is started on a daily dosing regimen, which the octreotide will be increased to twice or three times daily if clinical effects are not achieved using a single daily dose. Within days from the initiation of daily dosing, the patient experiences relief from headache. Intracranial pressure is also found to be reduced. No side effects are associated with the administration.

### Example 10. Treatment of Cluster Headache Using Octreotide Nose-to-Brain Liquid Formulation

A patient suffering from cluster headaches without relief from any therapy during or between episodes is started on a course of treatment with an intranasal formulation of octreotide acetate delivered using a nose-to-brain delivery device, and topiramate administered orally. The formulation is octreotide 1.0 mg/mL, PEG 400 2% (w/v), polysorbate 80 2% (w/v), BAC 0.02% (w/v) in 0.01M citrate buffer pH 4.5, The tonicity is increased with NaCl; delivery is using a VP7-232NE device (Aptar Pharma). The device delivers 100 mcl per pump. The dose is 100 mcg per pump. Topiramate is administered at a dose of 25 mg daily. Treatment is started at the onset of a headache cycle. The patient is started on a once daily dosing regimen, which will be increased to twice or three times daily if clinical effects are not achieved using a single daily dose. Within one week from the initiation of daily dosing, the patient experiences a reduction in frequency of headaches which breaks the attack cycle. The patient is maintained on a less frequent dosing schedule which prevents recrudescence.

### Example 11. Treatment of Idiopathic Intracranial Hypertension Using Octreotide Nose-to-Brain Powder Formulation

A patient suffering from idiopathic intracranial hypertension is started on a course of treatment with an intranasal formulation of octreotide acetate delivered using a nose-to-brain delivery formulation and device, and acetazolamide administered orally. The formulation is octreotide 0.1%, mannitol 95%, hydrogenated lecithin 5%. An Aptar UDSp device is used for delivery; each pump delivers 10 mg. The dose is 10 mcg per pump. Acetazolamide is administered at 125 mg once a day, orally. The patient is started on a daily dosing regimen, which will be increased to twice or three times daily if clinical effects are not achieved using a single daily dose. Within days from the initiation of daily dosing, the patient experiences relief from headache. Intracranial pressure is also found to be reduced. No side effects are associated with the administration.

### Example 12. Treatment of Cluster Headache Using Octreotide Nose-to-Brain Powder Formulation

A patient suffering from cluster headaches without relief from any therapy during or between episodes is started on a course of treatment with an intranasal formulation of octreotide acetate delivered using a nose-to-brain delivery device, and topiramate administered orally. The formulation is octreotide 0.5%, mannitol 95%, hydrogenated lecithin 5%. The tonicity is increased with NaCl. An Aptar UDSp device is used for delivery; each pump delivers 50 mg. The dose is 250 mcg per pump. Treatment is started at the onset of a headache cycle. Topiramate is administered at a dose of 25 mg daily. The patient is started on a daily dosing regimen, which will be increased to twice or three times daily if clinical effects are not achieved using a single daily dose. Within one week from the initiation of daily dosing, the patient experiences a reduction in frequency of headaches which breaks the attack cycle. The patient is maintained on a less frequent dosing schedule which prevents recrudescence.

What is claimed is:

1. A method for treating idiopathic intracranial hypertension (IIH) in a subject in need thereof, comprising administering to the subject a therapeutically effective combination of:
(a) an intranasal dosing regimen of a somatostatin mimetic formulated for direct nose-to-brain administration, wherein the dosing regimen of the somatostatin mimetic provides a dose level or exposure that is less than the therapeutically effective dosing regimen when administered alone by a non-nasal route of administration; and
(b) a dosing regimen of a carbonic anhydrase inhibitor, wherein the dosing regimen of a carbonic anhydrase inhibitor provides a dose level or exposure that is less than the therapeutically effective dose when administered alone.

2. A method for treating cluster headache in a subject in need thereof, comprising administering to the subject a therapeutically effective combination of:
(a) an intranasal dosing regimen of a somatostatin mimetic formulated for direct nose-to-brain administration, wherein the dosing regimen of the somatostatin mimetic provides a dose level or exposure that is less than the therapeutically effective dosing regimen when administered alone by a non-nasal route of administration; and
(b) a dosing regimen of a carbonic anhydrase inhibitor, wherein the dosing regimen of a carbonic anhydrase inhibitor provides a dose level or exposure that is less than the therapeutically effective dose when administered alone.

3. The method of claim 1 or 2 wherein the dosing regimen of the carbonic anhydrase inhibitor elicits fewer or no side effects than that elicited by the therapeutically effective dose when administered alone.

4. The method of claim 1 or 2 wherein a serum area-under-the-curve from a unit dose of the intranasal amount of the somatostatin mimetic is less than the serum area-under-the-curve of an effective unit dose of the somatostatin mimetic administered by a non-intranasal route.

5. The method of claim 1 or 2 wherein a serum area-under-the-curve from a unit dose of the intranasal amount of the somatostatin mimetic is less than the serum area-under-the-curve from an effective unit dose of the somatostatin mimetic administered by the intranasal route for an indication other than IIH or cluster headache.

6. The method of claim 5 wherein the indication other than IIH or cluster headache is acromegaly or carcinoid syndrome.

7. The method of claim 1, 2 or 4 wherein the non-intranasal route is subcutaneous, intramuscular, oral or intravenous.

8. The method of claim 1 or 2 wherein the effective intranasal amount of somatostatin mimetic is about equal to or less than about 100 mcg daily.

9. The method of claim 4 wherein the serum area-under-the-curve of the intranasal amount of the somatostatin mimetic is about equal to or less than about 50% of the serum area-under-the-curve of the somatostatin mimetic administered by a non-intranasal route.

10. The method of claim 4 wherein the serum area-under-the-curve of the intranasal amount of the somatostatin mimetic is about equal to or less than about 25% of the serum area-under-the-curve of the somatostatin mimetic administered by a non-intranasal route.

11. The method of claim 4 wherein the serum area-under-the-curve of the intranasal amount of the somatostatin mimetic is about equal to or less than about 10% of the serum area-under-the-curve of the somatostatin mimetic administered by a non-intranasal route.

12. The method of claim 5 wherein the serum area-under-the-curve of the intranasal amount of the somatostatin mimetic is about equal to or less than about 50% of the serum area-under-the-curve of the somatostatin mimetic administered by the intranasal route for an indication other than IIH or cluster headache.

13. The method of claim 5 wherein the serum area-under-the-curve of the intranasal amount of the somatostatin mimetic is about equal to or less than about 25% of the serum area-under-the-curve of the somatostatin mimetic administered by the intranasal route for an indication other than IIH or cluster headache.

14. The method of claim 5 wherein the serum area-under-the-curve of the intranasal amount of the somatostatin mimetic is about equal to or less than about 10% of the serum area-under-the-curve of the somatostatin mimetic administered by the intranasal route for an indication other than IIH or cluster headache.

15. The method of claim 1 or 2 wherein the effective intranasal amount of somatostatin mimetic is administered as a single daily dose, twice a day dosing, three times a day dosing, four times a day dosing, or as 2, 3 or 4 divided doses.

16. The method of claim 1 or 2 wherein the effective intranasal amount is administered daily for a duration effective to treat or resolve the IIH or cluster headache.

17. The method of claim 1 or 2, wherein the somatostatin mimetic is selected from somatostatin, octreotide, lanreotide, pasireotide, pentetreotide, or any combination thereof.

18. The method of claim 1 or 2 wherein the somatostatin mimetic formulated for intranasal administration comprises a powder, liquid or gel.

19. The method of claim 1 or 2 wherein the effective intranasal amount of somatostatin mimetic provides a minimal effective dose.

20. The method of claim 1 or 2 wherein treating is to reduce or prevent an acute, ongoing episode, and/or prophylactic and/or maintenance therapy to prevent future episodes.

21. The method of claim 1 or 2 wherein a carbonic anhydrase inhibitor is administered orally.

22. The method of claim 1 or 2 wherein a carbonic anhydrase inhibitor is administered once daily or twice daily.

23. The method of claim 1 or 2 wherein the therapeutically effective combination is a synergistic combination.

24. The method of claim 1 or 2 wherein the carbonic anhydrase inhibitor is selected from among topiramate, acetazolamide, methazolamide, zonisamide, sulthiame, bichlorphenamide, or a combination thereof.

25. The method of claim 1 or 2 wherein the dose level, dose frequency, or combination thereof of a carbonic anhydrase inhibitor when administered alone elicits fewer side effects than elicited by the dose level, dose frequency or combination thereof that is effective to treat approved indications.

26. The method of claim 1 or 2 wherein the dose level, dose frequency, or combination thereof of a carbonic anhydrase inhibitor when administered alone elicits fewer side effects than elicited by the dose level, dose frequency or combination thereof that is effective to treat IIH or cluster headache.

27. The method of claim 1 or 2 wherein the carbonic anhydrase inhibitor is acetazolamide and the dosing regimen is equal to or less than about 500 mg once a day or twice a day.

28. The method of claim 27 wherein the dosing regimen is about 250 mg once a day or twice a day.

29. The method of claim 28 wherein the dosing regimen is about 125 mg once a day or twice a day.

30. The method of claim 1 or 2 wherein the carbonic anhydrase inhibitor is topiramate and the dosing regimen is equal to or less than about 50 mg once a day or twice a day.

31. The method of claim 30 wherein the dosing regimen of topiramate is about 25 mg once a day or twice a day.

32. The method of claim 30 wherein the dosing regimen of topiramate is about 15 mg once a day or twice a day.

33. A therapeutic combination for treatment of idiopathic intracranial hypertension (IIH), comprising
(a) one or more unit doses of a somatostatin mimetic formulated for intranasal administration, wherein the dosing regimen of the somatostatin mimetic unit dose provides a dose level or exposure that is less than the therapeutically effective dosing regimen when administered alone by a non-nasal route of administration;
(b) one or more unit doses of a carbonic anhydrase inhibitor, wherein the dosing regimen of a unit dose level of a carbonic anhydrase inhibitor provides a dose or exposure that is less than the therapeutically effective dose when administered alone; and
(c) instructions for individual administration of each of the somatostatin mimetic and a carbonic anhydrase inhibitor in accordance with an effective dosing regimen.

34. A therapeutic combination for treatment of cluster headache, comprising
(a) one or more unit doses of a somatostatin mimetic formulated for intranasal administration, wherein the dosing regimen of the somatostatin mimetic unit dose provides a dose level or exposure that is less than the therapeutically effective dosing regimen when administered alone by a non-nasal route of administration;
(b) one or more unit doses of a carbonic anhydrase inhibitor, wherein the dosing regimen of a unit dose level of a carbonic anhydrase inhibitor provides a dose or exposure that is less than the therapeutically effective dose when administered alone; and
(c) instructions for individual administration of each of the somatostatin mimetic and a carbonic anhydrase inhibitor in accordance with an effective dosing regimen.

35. The therapeutic combination of claim 33 or 34 wherein the dosing regimen of the carbonic anhydrase inhibitor elicits fewer or no side effects than that elicited by the therapeutically effective dose when administered alone.

36. The therapeutic combination of claim 33 or 34 wherein a serum area-under-the-curve of a unit dose of the somatostatin mimetic administered intranasally is less than the serum area-under-the-curve of the unit dose of the somatostatin mimetic administered by a non-intranasal route.

37. The therapeutic combination of claim 33 or 34 wherein the non-intranasal route is subcutaneous, intramuscular oral or intravenous.

38. The therapeutic combination of claim 33 or 34 wherein a unit dose of the somatostatin mimetic formulated for intranasal administration is about equal to or less than about 100 mcg.

39. The therapeutic combination of claim 33 or 34 wherein a unit dose of the somatostatin mimetic formulated for intranasal administration is about equal to or less than about 50 mcg.

40. The therapeutic combination of claim 33 or 34 wherein a unit dose of the somatostatin mimetic formulated for intranasal administration is about equal to or less than about 10 mcg.

41. The therapeutic combination of claim 33 or 34 wherein a unit dose of the somatostatin mimetic formulated for intranasal administration is about equal to or less than about 5 mcg.

42. The therapeutic combination of claim 33 or 34 wherein a unit dose of the somatostatin mimetic formulated for intranasal administration is equal to about 1 mcg.

43. The therapeutic combination of claim 33 or 34 wherein the serum area-under-the-curve of a unit dose of the somatostatin mimetic administered intranasally is about equal to or less than about 50% of the serum area-under-the-curve of a unit dose of the somatostatin mimetic administered by a non-intranasal route.

44. The therapeutic combination of claim 33 or 34 wherein the serum area-under-the-curve of a unit dose of the somatostatin mimetic administered intranasally is about equal to or less than about 25% of the serum area-under-the-curve of a unit dose of the somatostatin mimetic administered by a non-intranasal route.

45. The therapeutic combination of claim 33 or 34 wherein the serum area-under-the-curve of a unit dose of the somatostatin mimetic administered intranasally is about equal to or less than about 10% of the serum area-under-the-curve of a unit dose of the somatostatin mimetic administered by a non-intranasal route.

46. The therapeutic combination of claim 33 or 34 wherein a serum area-under-the-curve from a unit dose of the intranasal amount of the somatostatin mimetic is less than the serum area-under-the-curve from an effective single dose of the somatostatin mimetic administered by the intranasal route for an indication other than IIH or cluster headache.

47. The therapeutic combination of claim 46 wherein the indication other than IIH or cluster headache is acromegaly or carcinoid syndrome.

48. The therapeutic combination of claim 33 or 34 wherein the unit dose is labeled for administration in a single daily dose, twice a day dosing, three times a day dosing, four times a day dosing, or as 2, 3 or 4 divided doses.

49. The therapeutic combination of claim 33 or 34 wherein the unit dose is labeled for daily administration for a duration effective to treat or resolve the IIH or cluster headache.

50. The therapeutic combination of claim 33 or 34 wherein treatment is to reduce or prevent an acute, ongoing episode, and/or prophylactic and/or maintenance therapy to prevent future episodes.

51. The therapeutic combination of claim 33 or 34 wherein the somatostatin mimetic is selected from somatostatin, octreotide, lanreotide, pasireotide, pentetreotide, or any combination thereof.

52. The therapeutic combination of claim 33 or 34 wherein the somatostatin mimetic formulated for intranasal administration comprises a powder, liquid or gel.

53. The therapeutic combination of claim 33 or 34 wherein the unit dose is a minimal effective dose.

54. The therapeutic combination of claim 33 or 34 wherein a carbonic anhydrase inhibitor is administered orally.

55. The therapeutic combination of claim 33 or 34 wherein a carbonic anhydrase inhibitor is administered once daily or twice daily.

56. The therapeutic combination of claim 33 or 34 wherein the dose level, dose frequency, or combination thereof of a carbonic anhydrase inhibitor is lower than effective to treat approved indications.

57. The therapeutic combination of claim 33 or 34 wherein the carbonic anhydrase inhibitor is selected from among topiramate, acetazolamide, methazolamide, zonisamide, sulthiame, bichlorphenamide, or a combination thereof.

58. The therapeutic combination of claim 33 or 34 wherein the carbonic anhydrase inhibitor is acetazolamide and the dosing regimen is equal to or less than about 500 mg once a day or twice a day.

59. The therapeutic combination of claim 58 wherein the dosing regimen is about 250 mg once a day or twice a day.

60. The therapeutic combination of claim 58 wherein the dosing regimen is about 125 mg once a day or twice a day.

61. The therapeutic combination of claim 33 or 34 wherein the carbonic anhydrase inhibitor is topiramate and the dosing regimen is equal to or less than about 50 mg once a day or twice a day.

62. The therapeutic combination of claim 61 wherein the dosing regimen of topiramate is about 25 mg once a day or twice a day.

63. The therapeutic combination of claim 61 wherein the dosing regimen of topiramate is about 15 mg once a day or twice a day.

64. The therapeutic combination of claim 33 or 34 which is a synergistic combination.

65. The therapeutic combination of claim 33 or 34 wherein the dose level, dose frequency, or combination thereof of a carbonic anhydrase inhibitor when administered alone in accordance with the instructions elicits fewer side effects than elicited by the dose level, dose frequency or combination thereof that is effective to treat approved indications.

66. The therapeutic combination of claim 33 or 34 wherein the dose level, dose frequency, or combination thereof of a carbonic anhydrase inhibitor when administered alone in accordance with the instructions elicits fewer side effects than elicited by the dose level, dose frequency or combination thereof that is effective to treat IIH or cluster headache.

67. A therapeutic combination for treatment of idiopathic intracranial hypertension (IIH) or cluster headache, comprising
(a) one or more unit doses of a somatostatin mimetic formulated for intranasal administration;
(b) one or more unit doses of a carbonic anhydrase inhibitor; and
(c) instructions for individual administration of each of the somatostatin mimetic and a carbonic anhydrase inhibitor in accordance with an effective dosing regimen;
wherein the unit dose of a carbonic anhydrase inhibitor administered in accordance with the instructions elicits fewer side effects than the unit dose of a carbonic anhydrase inhibitor as administered alone for treatment of approved indications.

68. A therapeutic combination for treatment of idiopathic intracranial hypertension (IIH) or cluster headache, comprising
(a) one or more unit doses of a somatostatin mimetic formulated for intranasal administration;
(b) one or more unit doses of a carbonic anhydrase inhibitor; and
(c) instructions for individual administration of each of the somatostatin mimetic and a carbonic anhydrase inhibitor in accordance with an effective dosing regimen;
wherein the unit dose of a carbonic anhydrase inhibitor administered in accordance with the instructions elicits fewer side effects than the unit dose of a carbonic anhydrase inhibitor as administered alone for treatment of idiopathic intracranial hypertension or cluster headache.

69. The method of claim 1 or 2, wherein the somatostatin mimetic is octreotide, and wherein the octreotide comprises a liquid pharmaceutical composition comprising:
octreotide 0.1 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

70. The method of claim 69 wherein the microcrystalline cellulose/sodium carboxymethycellulose comprises microcrystalline cellulose with about 11.3 to 18.8% sodium carboxymethylcellulose.

71. The method of claim 1 or 2 wherein the somatostatin mimetic is octreotide and wherein the octreotide comprises a liquid pharmaceutical composition comprising
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

72. The method of claim 1 or 2 wherein the somatostatin mimetic is octreotide and the octreotide comprises a liquid pharmaceutical composition consisting essentially of:
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

73. The method of claim 71 or 72 further comprising a tonicity adjusting agent such as mannitol, dextrose, sodium chloride, sorbitol or fructose, or any combination thereof, such that the tonicity is about 290 to about 500 mOsm/kg.

74. The method of claim 1 or 2 wherein the somatostatin mimetic is octreotide and the octreotide comprises a powder pharmaceutical composition comprising
octreotide 0.1%, mannitol 95%, lecithin 5%; or
octreotide 0.5%, mannitol 95%, lecithin 5%; or
octreotide 0.1%, calcium carbonate 95%, mannitol 2.5%, L-leucine 2.5%; or
octreotide 0.5%, calcium carbonate 95%, mannitol 2.5%, L-leucine 2.5%.

75. The method of claim 1 or 2 wherein the somatostatin mimetic is octreotide and the octreotide comprises a powder pharmaceutical composition consisting essentially of:
octreotide 0.1%, mannitol 95%, lecithin 5%; or
octreotide 0.5%, mannitol 95%, lecithin 5%; or
octreotide 0.1%, calcium carbonate 95%, mannitol 2.5%, L-leucine 2.5%; or
octreotide 0.5%, calcium carbonate 95%, mannitol 2.5%, L-leucine 2.5%.

76. The method or therapeutic combination of any one of claims 74-75 wherein the powder pharmaceutical composition is prepared by spray drying, optionally at a temperature of 100° C. or 120° C.

77. The therapeutic combination of any one of claim 33, 34, 67 or 68, wherein the somatostatin mimetic is octreotide, and wherein the octreotide comprises a liquid pharmaceutical composition comprising:
octreotide 0.1 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, microcrystalline cellulose/sodium carboxymethycellulose 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

78. The therapeutic combination of claim 77, wherein the microcrystalline cellulose/sodium carboxymethycellulose comprises microcrystalline cellulose with about 11.3 to 18.8% sodium carboxymethylcellulose.

79. The therapeutic combination of any one of claim 33, 34, 67 or 68, wherein the somatostatin mimetic is octreotide and wherein the octreotide comprises a liquid pharmaceutical composition comprising
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

80. The therapeutic combination of any one of claim 33, 34, 67 or 68, wherein the somatostatin mimetic is octreotide and the octreotide comprises a liquid pharmaceutical composition consisting essentially of:
octreotide 0.1 mg/mL, Avicel CL611 2% (w/v), polysorbate 80 2% (w/v), BAC 0.02% (w/v) in 0.01M citrate buffer pH 4.5; or
octreotide 1.0 mg/mL, Avicel CL611 2% (w/v), polysorbate 80 2% (w/v), BAC 0.02% (w/v) in 0.01M citrate buffer pH 4.5; or
octreotide 1.0 mg/mL, PEG 400 2% (w/v), polysorbate 80 2% (w/v), BAC 0.02% (w/v) in 0.01M citrate buffer pH 4.5; or
octreotide 0.1 mg/mL, PEG 400 2% (w/v), polysorbate 80 2% (w/v), BAC 0.02% (w/v) in 0.01M citrate buffer pH 4.5.

81. The therapeutic combination of any one of claim 79 or 80 further comprising a tonicity adjusting agent such as mannitol, dextrose, sodium chloride, sorbitol or fructose, or any combination thereof, such that the tonicity is about 290 to about 500 mOsm/kg.

82. The therapeutic combination of any one of claim 33, 34, 67 or 68, wherein the somatostatin mimetic is octreotide and the octreotide comprises a powder pharmaceutical composition comprising
octreotide 0.1%, mannitol 95%, lecithin 5%; or
octreotide 0.5%, mannitol 95%, lecithin 5%; or
octreotide 0.1%, calcium carbonate 95%, mannitol 2.5%, L-leucine 2.5%; or
octreotide 0.5%, calcium carbonate 95%, mannitol 2.5%, L-leucine 2.5%.

83. The therapeutic combination of any one of claim 33, 34, 67 or 68, wherein the somatostatin mimetic is octreotide and the octreotide comprises a powder pharmaceutical composition consisting essentially of:
octreotide 0.1%, mannitol 95%, lecithin 5%; or
octreotide 0.5%, mannitol 95%, lecithin 5%; or
octreotide 0.1%, calcium carbonate 95%, mannitol 2.5%, L-leucine 2.5%; or
octreotide 0.5%, calcium carbonate 95%, mannitol 2.5%, L-leucine 2.5%.

84. The therapeutic combination of claim 82 or 83, wherein the powder pharmaceutical composition is prepared by spray drying, optionally at a temperature of 100° C. or 120° C.
